(19) United States
(12) Patent Application Publication
(10) Pub. No.: US 2009/0170813 A1
(43) Pub. Date: Jul. 2, 2009

(54) Hypophosphorous Acid Derivatives and their Therapeutical Applications

(76) Inventors: Francine Acher, Vaucresson (FR); Chelliah Selvam, Paris (FR); Nicolas Triballeau, Paris (FR); Jean-Philippe Pin, Montpellier (FR); Hugues-Oliver Bertrand, Puteaux (FR)

Correspondence Address: NIXON & VANDERHYE, PC, 901 NORTH GLEBE ROAD, 11TH FLOOR, ARLINGTON, VA, 22203, US

(21) Appl. No.: 12/083,830

(22) PCT Filed: Oct. 18, 2006
(86) PCT No.: PCT/IB2006/003940

§ 371 Date: Nov. 12, 2008

Related U.S. Application Data
(60) Provisional application No. 60/727,449, filed on Oct. 18, 2005.
(60) Provisional application No. 60/743,483, filed on Mar. 15, 2006.

(51) Int. Cl. A61K 31/675; C07D 265/30; A61P 25/28

(52) U.S. Cl. 514/90; 544/157

(57) ABSTRACT

Hypophosphorous acid derivatives having Formula (I) wherein .M is a [C(R3,R4)]n1-C(E,COOR1,N(H,Z)) group, or an optionally substituted Ar—CH(COOR1,N(H,Z)) group, or an a, β, or a β, g-cyclic amino acid; .R₁ is H or R, R being an hydroxy or a carboxy protecting group; .Z is H or an amino protecting group R′, benzyl oxycarbonyl, benzyl or benzyl substituted; .E is H or a C1-C3 alkyl, aryl, an hydrophobic group; .R₂ is selected in the group comprising: D-CH(R₆)—C—(R₇,R₈), (R₁₁,R₁₂)CH—C(R₉,R₁₀), D-CH(OH), D-[C(R₁₃,R₁₄)]ₙ₃—, C[(R₁₅,R₁₆,R₁₇)]ₙ₄, D-CH₂, (R₁₈)CH═C(R₁₉), D-(M₁)ₙ₆—CO, Formula (II), PO(OH)₂—CH₂ or (PO(OH)₂—CH₂), (COOH—CH₂)—CH₂, with -D=H, OH, OR, (CH₂)ₙ₂OH, (CH₂)ₙ₁OR, COOH, COOR, (CH₂)ₙ₂COOH, (CH₂)ₙ₁COOR, SR, S(OR), SO₂R, NO₂, heteroaryl, C₁-C₃ alkyl, cycloalkyl, heterocycloalkyl, (CH₂)ₙ₂-alkyl, (COOH,NH₂)—(CH₂)ᵤ₁-cyclopropyl-(CH₂)ᵤ₂—, CO—NH-alkyl, Ar, (CH₂)ₙ₂—Ar, CO—NH—Ar; —R₃ to R₁₉ being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)n1-alkyl, aryl, (CH₂)n1-aryl, halogen, CF₃, SO₃H, (CH₂)ₓPO₃H₂, with x=0, 1 or 2, B(OH)₂, Formula (III), NO₂, SO₂NH₂, SO₂NHR; SR, S(O)R, SO₂R, benzyl; -M₁ is an alkylene or arylene group; -n1=1, 2 or 3, n2=1, 2 or 3, n3=0, 1, 2 or 3 and n4=1, 2 or 3, n5=1, 2 or 3, n6=0 or 1, u1 and u2, identical or different=0, 1 or 2, with the proviso that Formula (I) does not represent the racemic (3R,S) and the enantiomeric form (3R) of 3 amino,3-carboxy-propyl-2′-carboxy-ethylphosphinic acid; 3 amino,3-carboxy-propyl-4′carboxy,2′carboxy-butanoylphosphinic acid; 3 amino,3-carboxy-propyl-2′carboxy-butanoylphosphinic acid; 3 amino,3-carboxy-propyl-3′amino, 3′carboxy-propylylphosphinic acid; and 3 amino,3-carboxypropyl-7′amino-2′, 7′-dicarboxyheptylphosphinic acid, said hypophosphorous acid derivatives being diasteroisomers or enantiomers. Application as drugs.

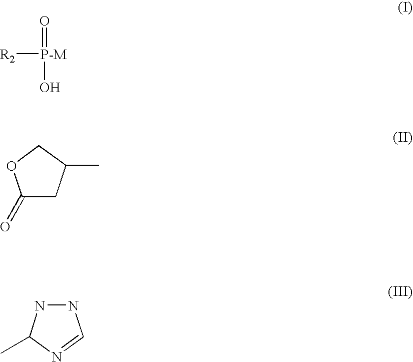

The invention relates to hypophosphorous acid derivatives having agonist or antagonist properties for metabotropic glutamate receptors (mGluRs), in particular agonist or antagonist properties for group III, subtype 4, metabotropic glutamate receptors (mGlu4Rs) and their therapeutical applications.

MGluRs are of particular interest in medicinal chemistry because they are believed to be suitable targets for treating a large variety of brain disorders such as convulsions, pain, drug addiction, anxiety disorders, and several neurodegenerative diseases.

The eight known subtypes of mGluRs are classified into three groups. Group III contains subtypes 4 and 6-8. Mainly located presynaptically, where they act as autoreceptors, group III mGluRs decrease adenylyl cyclase activity via a G_{1/10} protein and are specifically activated by L-AP4. Among this group, mGlu4R is thought to be a possible new target for Parkinson's disease, but the lack of a highly specific agonist has seriously impaired target validation studies. Furthermore, despite many chemical variations around the structure of glutamate, L-AP4 remains the strongest mGlu4R agonist with an EC₅₀ of only 0.32 μM and its α-methyl analogue, a competitive antagonist with an IC₅₀ of 100 μm. New chemotypes of higher potency and specificity are to be found.

The inventors' researches in that field lead them to develop methods of synthesis of hypophosphorous acids making it possible to obtain a large number of valuable agonists or antagonists for mGlu4Rs, particularly analogs of 3-amino-carboxy-propyl-2′-carboxy-ethylphosphinic acid (PCEP in short), with improved activity and selectivity compared to PCEP and valuable antagonists corresponding to the α-substituted derivatives thereof.

An object of the invention is then to provide new hypophosphorous acid derivatives, particularly having agonist or antagonist properties for group III mGluRs.

Another object of the invention is to provide new methods of synthesis of biologically active hypophosphorous acid derivatives with a large variety of substituents.

According to still another object, the invention takes advantage of the mGlu4Rs agonists or antagonist properties of the hypophosphorous acid derivatives thus obtained and aims to provide pharmaceutical compositions useful for treating brain disorders.

The hypophosphorous acid derivatives of the invention are diasteroisomers or enantiomers of formula (I)

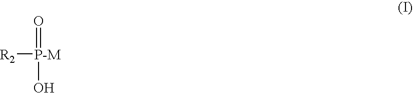

wherein
M is a [C(R₃,R₄)]ₙ₁—C,(E,COOR₁,N(H,Z)) group, or an optionally substituted Ar-CE,(COOR₁,N(H,Z)) group (Ar designating an aryl or an heteroaryl group), or an α, β, cyclic aminoacid group such as,

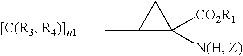

or a β,γ-cyclic aminoacid group such as

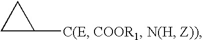

R₁ is H or R, R being an hydroxy or a carboxy protecting group, such as C₁-C₃ alkyl, Ar (being aryl or heteroaryl),
Z is H or an amino protecting group R′, such as C₁-C₃ alkyl, C₁-C₃ acyl, Boc, Fmoc, COOR, benzyl oxycarbonyl, benzyl or benzyl substituted such as defined with respect to Ar;
E is H or a C1-C3 alkyl, aryl, an hydrophobic group such as (CH₂)ₙ₁-alkyl, (CH₂)ₙ₁-aryl (or heteroaryl), such as a benzyl group, or a xanthyl, alkyl xanthyl or alkyl thioxanthyl group, or —(CH₂)ₙ₁-cycloalkyl, —(CH₂)ₙ—(CH₂—Ar)₂, a chromanyl group, particularly 4-methyl chromanyle, indanyle, tetrahydro naphtyl, particularly methyl-tetrahydronaphtyl;
   R₂ is selected in the group comprising:

### D-CH(R₆)—C—(R₇,R₈)—

### (R₁₁,R₁₂)CH—C(R₉,R₁₀)—

### D-CH(OH)—

D-[C(R₁₃,R₁₄)]ₙ₃—
C[(R₁₅,R₁₆,R₁₇)]ₙ₄—

### D-CH₂—

### (R₁₈)CH═C(R₁₉)—

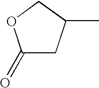

### PO(OH)₂—CH₂ or (PO(OH)₂—CH₂), (COOH—CH₂)—CH₂—

with
D=H, OH, OR, (CH₂)ₙ₂OH, (CH₂)ₙ₁OR, COOH, COOR, (CH₂)ₙ₂COOH, (CH₂)ₙ₁COOR, SR, S(OR), SO₂R, NO₂, heteroaryl, C₁-C₃ alkyl, cycloalkyl, heterocycloalkyl, (CH₂)ₙ₂-alkyl, (COOH,NH₂)—(CH₂)ᵤ₁-cyclopropyl-(CH₂)ᵤ₂—, CO—NH-alkyl, Ar, (CH₂)ₙ₂—Ar, CO—NH—Ar, R being as above defined and Ar being an optionally substituted aryl or heteroaryl group,
R₃ to R₁₉, identical or different, being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂,

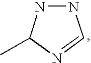

NO₂, SO₂NH₂, SO₂NHR; SR, S(O)R, SO₂R, benzyl;
   one of R₁₁ or R₁₂ being COOR, COOH, (CH₂)n₂-COOH, (CH₂)n₂-COOR, PO₃H₂ the other one being such as defined for R₉ and R₁₀;
one of R₁₅, R₁₆ and R₁₇ is COOH or COOR, the others, identical or different, being such as above defined;
one of R₁₈ and R₁₉ is COOH or COOR, the other being such as above defined;
M₁ is an alkylene or arylene group;
n1=1, 2 or 3;
n2=1, 2 or 3,
n3=0, 1, 2 or 3 and
n4=1, 2 or 3;
n5=1, 2 or 3;
n6=0 or 1,
u1 and u2, identical or different=0, 1 or 2,
   Ar, and alkyl groups being optionally substituted by one or several substituents on a same position or on different positions, said substituents being selected in the group comprising: OH, OR; (CH₂)ₙ₁OH, (CH₂)ₙ₁OR, COOH, COOR, (CH₂)ₙ₁COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂,

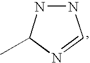

NO₂, SO₂NH₂, SO₂NHR; SR, S(O)R, SO₂R, benzyl;
R being such as above defined,
with the proviso that formula I does not represent the racemic (3R,S) and the enantiomeric form (3R) of 3 amino,3-carboxy-propyl-2′-carboxy-ethylphosphinic acid; 3 amino,3-carboxy-propyl-4′carboxy,2′carboxy-butanoylphosphinic acid; 3 amino,3-carboxy-propyl-2′carboxy-butanoylphosphinic acid; 3 amino,3-carboxy-propyl-3′amino, 3′carboxy-propylylphosphinic acid; and 3 amino,3-carboxypropyl-7′amino-2′,7′-dicarboxyheptylphosphinic acid.

In the above defined hypophosphorous acid derivatives of the invention, D is preferably Ar (optionally substituted), Ar—(CH₂)ₙ₂ (with Ar optionally substituted), C₁-C₃ alkyl or cycloalkyl; alkyl-(CH₂)ₙ₂, or COOH. Preferably Ar is a phenyl group (optionally substituted) or a carboxyalkyl group (optionally substituted). Alternatively, Ar is an heterocyclic group (optionally substituted). Advantageous groups are thiophenyl or furanyl group (optionally substituted).

A first preferred family corresponds to hypophosphorous acid derivatives of formula (II)

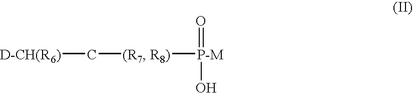

wherein the substituents are as above defined.

In particularly preferred derivatives of this family, D is Ar or a substituted Ar, especially a phenyl group optionally substituted by 1 to 5 substituents. The substituents are in ortho and/or meta and/or para positions. Preferred substituents comprise: OH, OR, (CH₂)₂ₙOH, (CH₂)ₙ₂OR, COOH, COOR, (CH₂)ₙ₂COOH, (CH₂)ₙ₂COOR, C1-C3 alkyl or cycloalkyl, (CH₂)ₙ₂-alkyl, aryl, (CH₂)ₙ₂-aryl, halogen, CF₃, SO₃H, PO₃H₂, B(OH)₂ alkylamino, fluorescent group (dansyl, benzoyl dinitro 3, 5′,

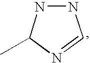

NO₂, SO₂NH₂, SO₂(NH,R)SR, S(O)R, SO₂R, OCF₃, heterocycle, heteroaryl, substituted such as above defined with respect to Ar.

[symbol]

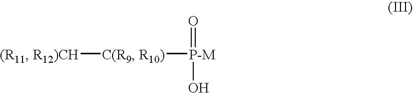

wherein the substituents are as above defined.

In preferred derivatives, one of R₁₁ or R₁₂ is COOH.

Advantageously, the other one of R₁₁ or R₁₂, and/or R₉ and/or R₁₀ are H, C₁-C₃ alkyl, OH, NH₂, CF₃.

A third preferred family corresponds to hypophosphorous acid derivatives of formula (IV)

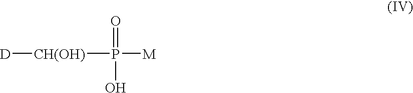

wherein the substituents are as above defined.

In preferred derivatives, D is as above defined with respect to formula (II)

In a fourth preferred family, the hypophosphorous acid derivatives have formula (V)

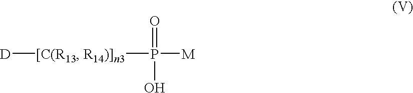

wherein the substituents are as above defined, one of R₁₃ or R₁₄ representing OH.

In preferred derivatives, D is as above defined with respect to formula (II).

The substituent R₁₃ or R₁₄ which does not represent OH is advantageously H, C₁-C₃ alkyl, OH, CF₃, NH₂.

In a fifth preferred family, the hypophosphorous acid derivatives have formula (VI)

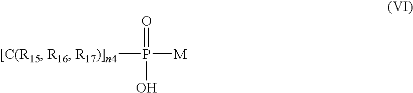

wherein the substituents are as above defined.

In preferred derivatives, in the first group of the chain, one or two of R₁₅, R₁₆ or R₁₇ are COOH, the other(s) advantageously being

[symbol]

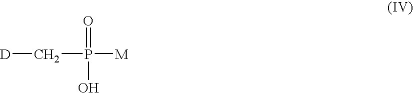

wherein the substituents are as above defined.

In preferred derivatives, D is as above defined with respect to formula (II).

In a seventh family, the hypophosphorous acid derivatives have formula (VIII)

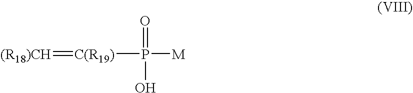

wherein the substituents are as above defined.

In preferred derivatives, R₁₈ is COOH.

Advantageously, R₁₉ is H, C₁-C₃ alkyl, OH.

An eighth family corresponds to hypophosphorous acid derivatives of formula (LIX)

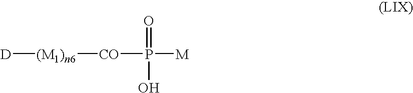

wherein the substituents are as above defined.

In preferred derivatives, either n6=0, or n6=1 and M₁ is an alkylene or arylene group such as above defined.

In a preferred embodiment of the invention M is a [C(R₃,R₄)]ₙ₁—C(E,COOR₁,N(H,Z)) group, in the above defined hypophosphorous acid derivatives.

Preferably R₃ and/or R₄ are H and n1=1 or 2, more preferably 2.

In another preferred embodiment, M is an Ar group or a substituted arylene group, particularly a C₆H₄ group or a substituted C₆H₄ group, the substituents being as above defined with respect to formula I.

In still another embodiment, M comprises a cyclic aminoacid group, particularly, M is an α, β cyclic aminoacid group such as

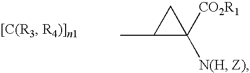

or a β,γ-cyclic aminoacid group such as

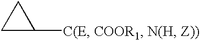

The invention particularly relates to the above mentioned derivatives wherein E represents H, which are group III mGluR agonists, and more particularly mGlu4R agonists of great interest.

The invention also particularly relates to the above mentioned derivatives wherein E is different from H and is more especially a C1-C3, alkyl, an aryl, an hydrophobic group such as a (CH₂)ₙ₁-alkyl group, or a (CH₂)ₙ₁-aryl group, as above defined, particularly a benzyl group, or a methylxanthyl group or alkylxanthyl or alkylthioxanthyl.

Advantageously, such derivatives are valuable mGluR antagonists, particularly mGlu4 antagonists.

The invention also relates to a process for preparing hypophosphorous acid derivatives of formula I

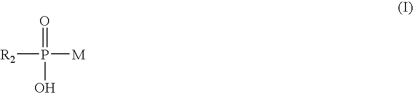

wherein the substituents are as above defined.

According to method A), said process comprises

a1) treating a derivative of formula (IX)

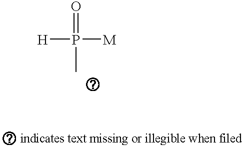

[symbol]

with either trimethylsilylchloride (TMSCl) and triethylamine (Et3N), or N,O-(bis-triethylsilyl)acetamide (BSA), (Et representing a C₂H₅ group).
a2) adding to the reaction product
one of the following derivatives having, respectively,

D-C(R₆)—C(R₇,R₈), or formula X

(R₁₁,R₁₂)C═C(R₉,R₁₀) formula XI

formula XII:

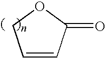

D-CH(═O) formula XIII

D-[C(R₁₃,R₁₄)]ₙ₃—Br formula XIV

[C(R₁₅,R₁₆,R₁₇)]ₙ₄—Br formula XV

D-I formula XVI

(R₁₈)C≡C(R₁₉) formula XVII

a3) treating the reaction product under acidic conditions or with catalysts to obtain the final desired product;
a4) recovering the diastereoisomers or the enantiomer forms,
a5) if desired, separating diastereoisomers, when obtained, into the enantiomers.

According to method B, said process comprises

b1) treating a derivative of formula (XVIII)

(R″SiO)₂—P—H (XVIII)

wherein R″ is a C₁-C₃ alkyl
with
   either a derivative of formula (X)

D-C(R₆)═C(R₇,R₈) (X)

or with a derivative of formula (XI)

(R₁₁,R₁₂)C═C(R₉,R₁₀) (XI)

wherein one of R₉ or R₁₀ is COOalk, alk being a C₁-C₃ alkyl
b2) treating the condensation product with a dibromo derivative of formula (XIX)

Br—[C(R₃,R₄)]ₙ₁—Br (XIX)

under reflux conditions; and adding HC(Oalk)₃
wherein alk is a C₁-C₃ alkyl
b3) treating the condensation product with a derivative of formula (XX)

NH(Z)-CH(CO₂R)₂ (XX)

in the presence of K₂CO₃, BuO₄NBr, under reflux conditions;
b4) treating the condensation product under acidic conditions or with catalyst to obtain the final desired product;
b5) recovering the diastereoisomers or the enantiomer forms, and
b6) if desired, separating diastereoisomers, when obtained, into the enantiomers.

Alternatively, the reaction product obtained at step b1) is reacted according to step b2i), with a derivative of formula (XXI)

[(R₃,R₄)C]ₙ₁═C(COOR₁,NH(Z)) (XXI)

In step b3i), the reaction product is treated under acidic conditions to give the final desired product.

According to method C, said process comprises

c1) reacting, as defined in step a1), a derivative of formula (XXII)

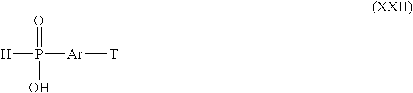

wherein Ar is as above defined and preferably an optionally substituted C₆H₄ group and T
c2) carrying out reaction step a2) by using one or the derivatives of formula (X) to (XVII)
c3) treating the reaction product with NBS, AIBN to have bromo derivatives with Ar substituted by T′-Br, with T′=CH₂
c4) reacting the bromo derivative thus obtained with (CH)₆ N₄ in an organic solvent, then AcOH/H₂O to obtain cetone derivatives with Ar substituted by —C═O;
c5) treating the cetone derivatives with KCN, NH₄Cl and NH₄OH to obtain aminocyano derivatives, with Ar substituted by —C(CN,NH₂),
c6) treating under acidic conditions to obtain derivatives with Ar substituted by —C(COOR,NH₂), and
c7) treating with catalysts to obtain the final desired product.

In method A, according to a preferred embodiment
the use of derivatives of formula (X)

D-CH(R₆)═C(R₇,R₈) (X)
with derivatives of formula (IX) results, in step a2), in intermediate derivatives of formula (XXIII)

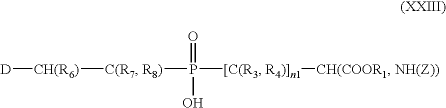

and, in step a3), in a final product of formula (XXIV)

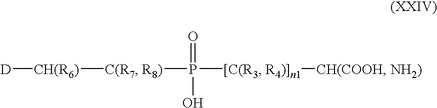

the use of derivatives of formula (XI) or formula (XII)

(R₁₁,R₁₂)C═C(R₉,R₁₀) (XI)

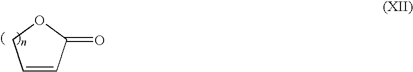

results, in step a2), in intermediate derivatives of formula (XXV)

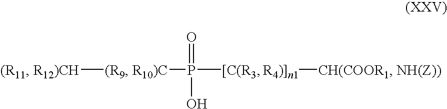

and, in step a3), in a final product of formula (XXVI)

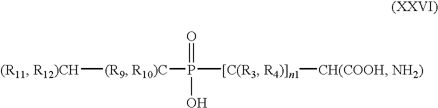

the use of derivatives of formula (XIII)

D-CH(═O) (XIII)
results, in step a2), in intermediate derivatives of formula (XXVII)

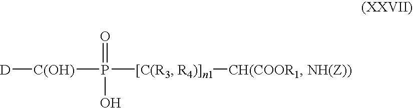

and, in step a3), in a final product of formula (XXVIII)

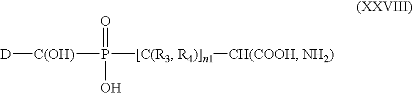

the use of derivatives of formula (XIV)

D-[C(R₁₃,R₁₄)]ₙ₃—Br (XIV)
results, in step a2), in intermediate derivatives of formula (XXIX)

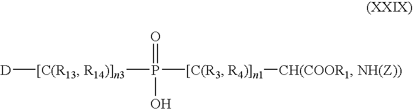

and, in step a3), in a final product of formula (XXX)

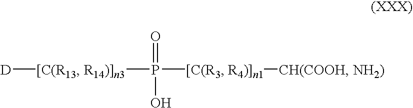

the use of derivatives of formula (XV)

[C(R₁₅,R₁₆,R₁₇)]ₙ₄—Br (XV)
results, in step a3), in intermediate derivatives of formula (XXXI)

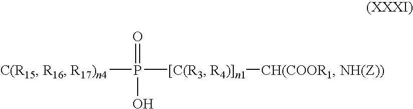

and, in step a3), in a final product of formula (XXXII)

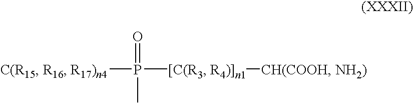

[symbol]

the use of derivatives of formula (XVI)

D-I (XVI)
results, in step a2), in intermediate derivatives of formula (XXXIII)

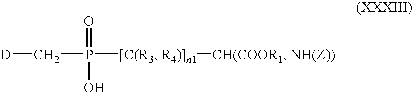

and, step a3), in a final product of formula (XXXIV)

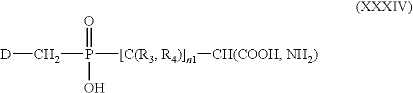

the use of derivatives of formula (XVII)

(R₈)C≡C(R₁₉) (XVII)
results, in step a2), in intermediate derivatives of formula (XXXV)

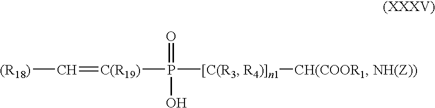

and, in step a3), in a final product of formula (XXXVI)

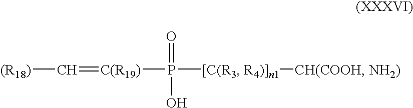

the use of derivatives of formula (LIX)

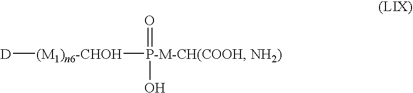

wherein M₁ is as above defined with respect to M and n6=0 or 1, and results by oxidation in a product of formula (LXI)

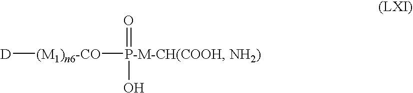

In method B,
the use, with derivatives of formula (XVIII),

(R″SiO)₂—P—H (XVIII)
of derivatives of formula (X)

D-CH(R₆)—C(R₇,R₈) (X)
results, in step b1), in intermediate derivatives of formula (XXXVII)

D-CH(R₆)—C(R₇,R₈)—P—(OSiR″)₂ (XXXVII)
in step b2), in intermediate derivatives of formula (XXXVIII)

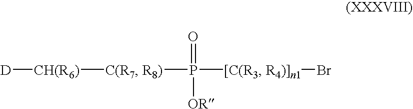

[symbol]

in step b3), in intermediate derivatives of formula (XXXIX)

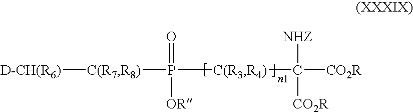

and, in step b4), in a final product of formula (XXXX)

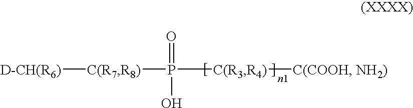

the use, with derivatives of formula (XVIII), of derivatives of formula (XI)

(R₁₁,R₁₂)C═C(R₉,R₁₀) (XI)
results, in step b1), in intermediate derivatives of formula (XXXXI)

(R₁₁,R₁₂)CH—C(R₉,R₁₀)—P—(OSiR″)₂ (XXXXI)
in step b2), in intermediate derivatives of formula (XXXXII)

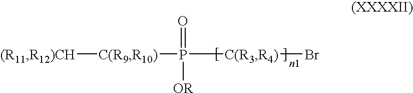

in step b3), in intermediate derivatives of formula (XXXXIII)

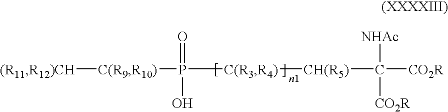

[symbol]

in step b4), in final products of formula (XXXXIV)

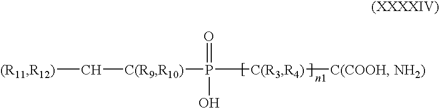

or, alternatively,
the use with derivatives of formula (XXXXI) obtained according to step b1)

(R₁₁,R₁₂)CH—C(R₉,R₁₀)—P—(OSiR″)₂ (XXXXI)
of derivatives of formula (XXXXV)

(R₃,R₄)C═C(COOR₁,NH(Z) (XXXXV)
results in intermediate derivatives of formula (XXXXVI)

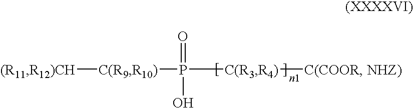

the treatment under acidic conditions giving the final product of formula (XXXXVII)

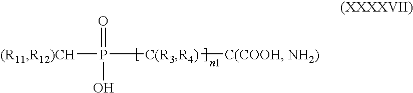

In method C,

the use, of a derivative of formula (XXII),

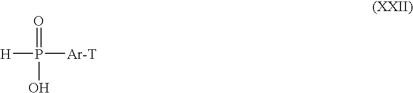

with a derivative of

D-C(R₆)═C(R₇, R₈), or formula X

(R₁₁,R₁₂)C═C(R₉,R₁₀) formula XI

formula XII:

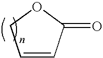

D-CH(═O) formula XIII

D-[C(R₁₃,R₁₄)]ₙ₃—Br formula XIV

[C(R₁₅,R₁₆,R₁₇)]ₙ₄—Br formula XV

D-I formula XVI

(R₁₈)C≡C(R₁₉) formula XVII

results in intermediate derivatives respectively having formulae (XXXXVIII) to (LIV)

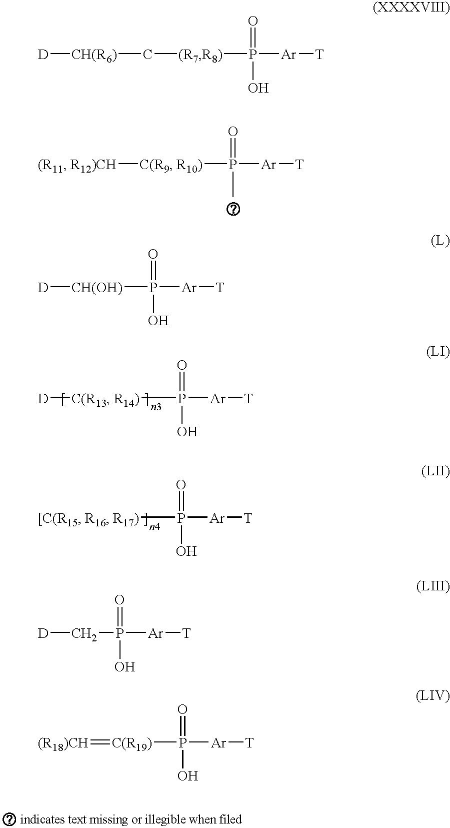

[symbol]

In method A, the derivatives of formula IX

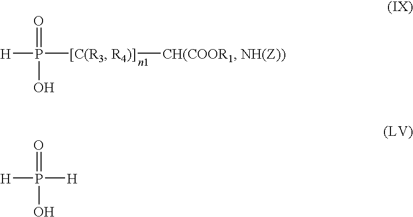

with a derivative of formula LVI

(R₃,R₄)ₙ₁C═CH—C(E,COOR₁,NH(Z)) (LVI)

Preferably, the derivative of formula (LVI) is Z-vinyl-glyOMe or a derivative thereof with E different from H, E being as above defined, and has formula (LVIa).

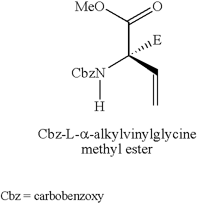

Z-vinyl-glyOMe is advantageously synthesized from methionine or glutamate according to references (1), (2) or (3).

Z-vinyl-glyOMe derivatives with E different from H can be prepared from α-alkyl methionine or alpha alkyl glutamate (see reference 4). Alpha amino acids can be stereoselectively α-alkylated using imidazolinones or oxazolidinones (references 5 and 6).

Other methods for obtaining Z-vinyl-glyOMe derivatives are given in Example 9. The reaction is advantageously carried out in the presence of AIBN by heating above 50° C.-100° C., preferably at about 80° C.

In method B, the derivatives of formula (XVIII)

(R″SiO)₂—P—H (XVIII)

are advantageously obtained by reacting an hypophosphorous acid ammonium salt of formula (LVII)

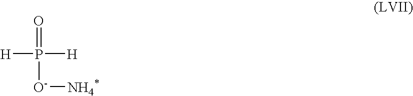

with a disilazane derivative of formula (LVIII)

(alk₃Si)₂—NH (LVIII)

The reaction is advantageously carried out under an inert gas, by heating above 100° C., particularly at about 120° C.,

or by reacting hypophosphorous acid with N,O-(bis-triethylsilyl)acetamide (BSA) at room temperature.

In method C, the derivatives of formula (XXII)

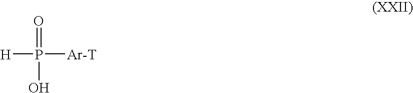

are advantageously obtained by reacting a mixture of H₃PO₂, Ar—NH₂, Ar—Br and a catalyst Pd(0) Ln. (Ln=n ligands).

According to method D, intermediate derivatives of formula

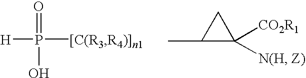

are prepared as disclosed in example 8.

The hypophosphorous acid derivatives which are intermediates in the above disclosed process, enter into the scope of the invention.

As above mentioned, said hypophosphorous acid derivatives have mGluRs agonist or antagonist properties of great interest and therefore are particularly valuable as active principles in pharmaceutical compositions to treat brain disorders.

They are particularly mGlu4Rs agonists or antagonists of great value.

The invention thus also relates to pharmaceutical compositions, comprising a therapeutically effective amount of at least one of the hypophosphorous acid derivatives of formula I in combination with a pharmaceutically acceptable carrier.

The invention also relates to the use of at least one of hypophosphorous acid derivatives of formula I for preparing a drug for treating brain disorders.

The pharmaceutical compositions and drugs of the invention are under a form suitable for an administration by the oral or injectable route.

For an administration by the oral route, compressed tablets, pills, capsules are particularly used. These compositions advantageously comprise 1 to 100 mg of active principle per dose unit, preferably 2.5 to 50 mg.

Other forms of administration include injectable solutions for the intravenous, subcutaneous or intramuscular route, formulated from sterile or sterilizable solution. They can also be suspensions or emulsions.

These injectable forms, for example, comprise 0.5 to 50 mg of active principle, preferably 1 to 30 mg per dose unit.

The pharmaceutical compositions of the invention prepared according to the invention are useful for treating convulsions, pain, drug addiction, anxiety disorders and neurodegenerative diseases.

By way of indication, the dosage which can be used for treating a patient in need thereof, for example, corresponds to doses of 10 to 100/mg/day, preferably 20 to 50 mg/day, administered in one or more doses.

The conditioning with respect to sale, in particular labelling and instructions for use, and advantageously packaging, are formulated as a function of the intended therapeutic use.

According to another object, the invention relates to a method for treating brain disorders, comprising administering to a patient in need thereof an effective amount of an hypophosphorous acid derivative such as above defined.

According to still another object, the invention relates to the use of at least one hypophosphorous acid derivative such as above defined for preparing a drug for treating drug disorders.

Other characteristics and advantages of the invention will be given in the following examples illustrating the synthesis of hypophosphorous acid derivatives. In the examples, it

[symbol]

### EXPERIMENTAL SECTION

### Example 1

### Synthesis of Hypophosphorous Acid Derivatives According to Method A

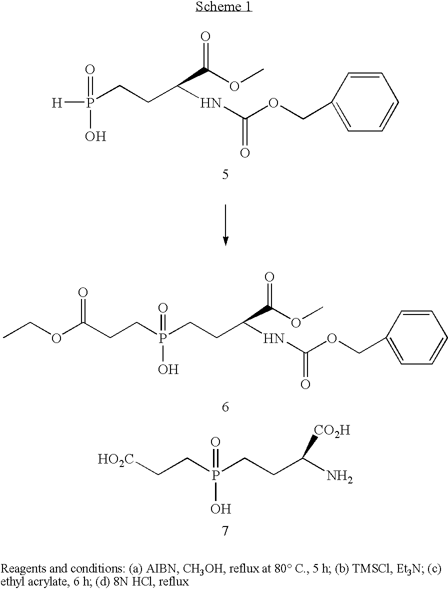

(3S)-3-[((3-(N-Benzyloxycarbonyl)amino-3-methoxycarbonyl)propyl)(hydroxy)phosphinyl] propanoic Acid Ethyl Ester (6). The compound 5 (180 mg, 0.57 mmol) was treated with a mixture of trimethylsilylchloride (TMSCl, 130.4 mg, 1.2 mmol)/triethylamine (Et₃N, 121 mg, 1.2 mmol) in dichloromethane at 0° C., then stirred at room temperature for one hour under a argon atmosphere. Then again cooled to 0° C. and ethyl acrylate was carefully added dropwise. The mixture was stirred at room temperature for 6 h. The reaction mixture was treated with 1N HCl and extracted with ethylacetate. The organic layer was dried over MgSO₄ and evaporated under vacuum to give 6 (130 mg, 55% yield). ¹H NMR (CD₃OD): δ 1.26 (t, J=7.0 Hz, 3H), 2.01 (m, 6H), 2.60 (m, 2H), 3.73 (s, 3H), 4.15 (q, J=7.0 Hz, 2H), 4.29 (m, 1H), 5.12 (s, 2H), 7.34 (m, 5H). ³¹P NMR (CD₃OD): δ 53.6.

(3S)-3-[((3-Amino-3-carboxy)propyl)(hydroxy)phosphinyl]propanoic Acid (7). A solution

[symbol]

NMR (D₂O): δ 1.69 (m, 2H), 1.89 (m, 2H), 2.08 (m, 2H), 2.55 (m, 2H), 4.00 (t, J=5.9 Hz, 1H). ³¹P NMR (D₂O): δ 57.4. [α]_{D}+12.8° (c 1.0, H₂O) (lit. [α]_{D}+12.5° (c 1.2, H₂O), Ragulin et al., JP-2001-213887).

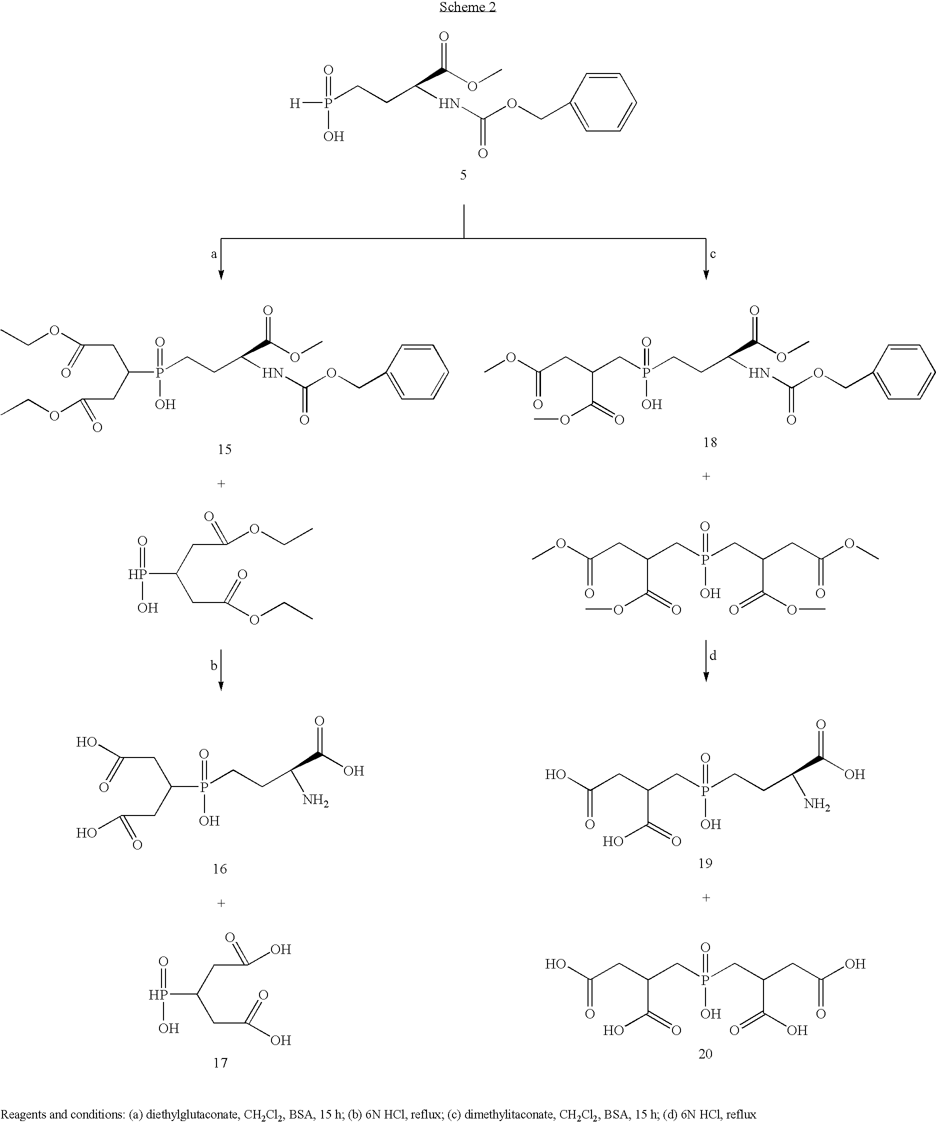

### Scheme 2a, 2b

(3S)-3-[(((3-(N-Benzyloxycarbonyl)amino-3-methoxycarbonyl)propyl)(hydroxy)phosphinyl)-methyl]pentane-1,5-dioic Acid Diethyl Ester (15). To a solution of 5 (0.8 mmol) and diethylglutaconate (558 mg, 3 mmol) in 2 ml of methylene chloride at 0° C. under an argon atmosphere was added dropwise N,O-(bis-triethysilyl)acetamide (BSA) (1.49 ml, 6 mmol). The mixture was allowed to warm to room temperature and stirred overnight, then cooled to 0° C. and 25 ml of 1N HCl were added, then extracted with ethyl acetate. The organic layer was concentrated in vacuo. This residue was dissolved in 10 ml of water, the pH was adjusted to 7 using saturated NaHCO₃ solution, then extracted with ethylacetate (2×50 ml). The organic layer was separated, and the aqueous phase was treated with 1N HCl to adjust the pH to 1. The aqueous phase was extracted with ethyl acetate twice (2×50 ml). The combined acidic organic extracts were dried over MgSO₄, filtered and concentrated in vacuo. ¹H NMR (CD₃OD): δ 1.26 (m, 6H), 2.40 (m, 9H), 3.74 (s, 3H), 4.13 (m, 4H), 4.37 (m, 1H), 5.12 (s, 2H), 7.37 (m, 5H). ³¹P NMR (CD₃OD): δ 54.80. ¹³C NMR (CD₃OD): δ 13.78, 23.89 (d, J=90.70 Hz), 24.11, 31.85 (d, J=93.75 Hz), 33.17, 52.13, 54.92, 60.62, 66.84, 128.00, 128.20, 128.66, 137.19, 157.43, 171.81, 172.18, 172.71.

(3S)-3-[(((3-amino-3-carboxy)propyl)(hydroxy)phosphinyl)-methyl]pentane-1,5-dioic Acid (16). Compound 15 was dissolved in 3 ml of 6N HCl. The mixture was heated for 15 h at reflux temperature, the resulting solution was cooled to room temperature. Volatile organic byproducts and water were removed under vacuo and the residue was purified using a Dowex AG50×4 column as described earlier (63% yield over two steps). ¹H NMR (D₂O): δ 1.63 (m, 2H); 2.00 (m, 2H); 2.33 (m, 3H); 2.58 (m, 2H); 3.91 (t, J=6.1 Hz, 1H). ³¹P NMR (D₂O): δ 57.10. ¹³C NMR (D₂O): δ 23.17, 23.42 (d, J=90.81 Hz), 31.84 (d, J=93.07 Hz), 33.62, 53.76 (d, J=14.6 Hz), 166.08, 172.16, 176.32 (d, J=12.26 Hz).

3-[((hydroxy)phosphinyl)-methyl]pentane-1,5-dioic Acid (17). The title compound was formed as a byproduct during the preparation of compound 15 and deprotected in the next step (procedure described above). During the deposition of 16, compound 17 was not bound to the cation exchange resin (Dowex AG50×4) and recovered. ¹H NMR (D₂O): δ 2.44 (m, 5H), 6.85 (d, J=568 Hz, 1H). ¹³C NMR (D₂O): δ 31.93, 31.97 (d, J=125.7 Hz), 175.41 (d, J=11.13 Hz).

### Scheme 2c, 2d

(3S)-2-[(((3-(N-Benzyloxycarbonyl)amino-3-methoxycarbonyl)propyl)(hydroxy)phosphinyl)-methyl]butane-1,4-dioic Acid Dimethyl Ester (18). The compound was prepared from 5 (0.8 mmol) and diethylitaconate (474 mg, 3 mmol) by a procedure similar to that for the preparation of compound 15 (61% yield). ¹H NMR (CD₃OD): δ 2.10 (m, 6H); 2.83 (m, 2H); 3.20 (m, 1H); 3.75 (s, 3H); 3.72 (s, 3H); 3.71 (s, 3H); 4.30 (m, 1H); 5.13 (s, 2H) 7.38 (m, 5H). ³¹P NMR (CD₃OD): δ 52.21. ¹³C NMR (CD₃OD): δ 24.26, 25.88 (d, J=93.39 Hz), 29.93 (d, J=93.39 Hz), 35.85, 36.27, 51.53, 52.05, 54.13, 54.62, 66.82, 127.99, 128.20, 128.64, 137.20, 157.47, 172.42, 172.71, 174.56 (d, J=9.94 Hz).

(3S)-2-[(((3-amino-3-carboxy)propyl)(hydroxy)phosphinyl)-methyl]butane-1,4-dioic Acid (19). The compound was prepared from 18 by the removal of protecting groups following the same procedure as that followed for compound 16 and purified by Dowex AG50×4-column to afford 19 (quantitative yield). ¹H NMR (D₂O): δ 1.82 (m, 3H); 2.17 (m, 3H); 2.78 (m, 2H); 3.08 (m, 1H); 4.04 (t, J=6.1 Hz, 1H). ³¹P NMR (D₂O): δ 56.35. ¹³C NMR (D₂O): δ 23.27, 25.49 (d, J=91.38 Hz), 30.34 (d, J=91.25 Hz), 36.02, 36.95 (d, J=7.61 Hz), 53.71 (d, J=15.09 Hz), 172.12, 175.92, 178.28 (d, J=8.81 Hz).

(3S)-2-[(hydroxy)phosphinyl]-bismethylbutane-1,4-dioic Acid (20). The title compound was formed as a byproduct during the preparation of compound 18 and then deprotected in the next step (procedure described above). During the deposition of 19 on cation exchange resin, the compound 20 was not bound to the resin. ¹H NMR (D₂O): δ 1.84 (m, 2H); 2.08 (m, 2H); 2.56 (d, J=6.69 Hz, 4H); 2.92 (m, 2H). ³¹P NMR (D₂O): δ 62.83. ¹³C NMR (D₂O): δ 30.26 (d, J=91.88 Hz), 35.49, 36.69, 175.46, 177.52 (d, J=8.74 Hz).

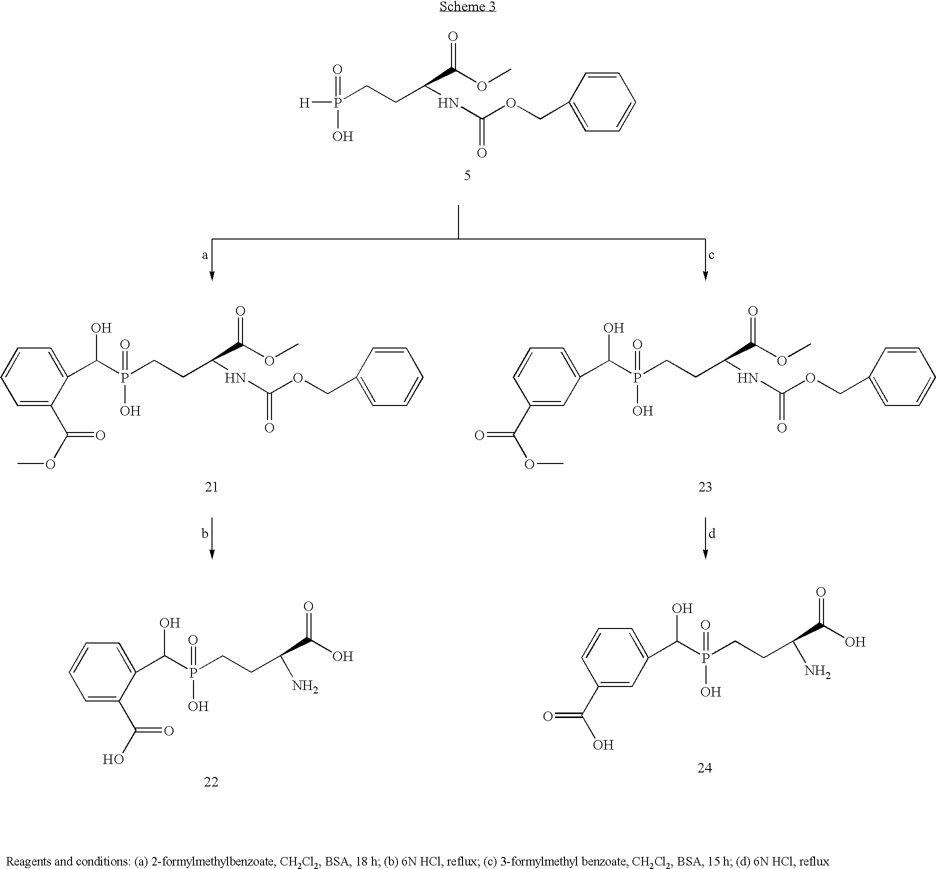

(3S)-2-[(((3-(N-Benzyloxycarbonyl)amino-3-methoxycarbonyl)propyl)(hydroxy)phosphinyl)-hydroxymethyl]benzoic Acid Methyl Ester (21). The compound was prepared from 5 (0.8 mmol) and ethyl 2-formylbenzoate (492 mg, 3 mmol) by using the procedure described for preparation of compound 15 (37% yield). ¹H NMR (CD₃OD): δ 1.84 (m, 2H); 2.22 (m, 2H); 3.73 (s, 3H); 3.88 (s, 3H); 4.32 (m, 1H); 5.10 (s, 2H); 5.84 (d, J=8.12 Hz, 1H); 7.32 (m, 5H); 7.70 (m, 4H). ³¹P NMR (CD₃OD): δ 41.22.

(3S)-2-[(((3-amino-3-carboxy)propyl)(hydroxy)phosphinyl)-hydroxymethyl]benzoic Acid (22). The removal of the protecting groups in compound 21 was accomplished following the same procedure as that followed for compound 16 and purified by anion exchange AG1×4 column using the procedure as described for compound 10. The compound 22 was eluted with 0.4-0.5M HCOOH (quantitative yield). ¹H NMR (D₂O): δ 1.67 (m, 2H), 2.08 (m, 2H), 3.98 (m, 1H), 5.74 (d, J=6.43 Hz, 1H), 7.63 (t, J=7.65 Hz, 1H), 7.69 (d, J=7.66 Hz, 1H), 7.79 (t, J=7.17 Hz, 1H), 7.91 (d, J=7.63 Hz, 1H). ³¹P NMR (D₂O): δ 43.20. ¹³C NMR (D₂O): δ 22.53 (d, J=89.68 Hz), 23.55, 54.16, 72.92 (d, J=107.11 Hz), 128.22, 129.13, 129.38, 130.10, 132.43, 138.77, 170.82, 172.49.

(3S)-3-[(((3-(N-Benzyloxycarbonyl)amino-3-methoxycarbonyl)propyl)(hydroxy)phosphinyl)-hydroxymethyl]benzoic Acid Methyl Ester (23). The compound was prepared from 5 (0.8 mmol) and methyl 3-formylbenzoate (492 mg, 3 mmol) by using the procedure described for preparation of compound 15 (55% yield). ¹H NMR (CD₃OD): δ 1.79 (m, 4H), 3.60 (s, 3H), 3.76 (s, 3H), 4.26 (m, 1H), 4.95 (m, 1H), 5.02 (s, 2H), 7.30 (m, 5H), 7.83 (m, 4H). ³¹P NMR (CD₃OD): δ 53.09, 53.53.

(3S)-3-[(((3-amino-3-carboxy)propyl)(hydroxy)phosphinyl)-hydroxymethyl]benzoic Acid (24). The removal of the protecting groups in compound 21 was accomplished following the same procedure as that followed for compound 16 and purified by Dowex AG50×4 column to afford 24 (quantitative yield). ¹H NMR (D₂O): δ 1.77 (m, 2H), 2.11 (m, 2H), 3.99 (m, 1H), 4.93 (d, J=9.45 Hz, 1H), 7.50 (t, J=7.73 Hz, 1H), 7.66 (d, J=7.46 Hz, 1H), 7.92 (d, J=7.57 Hz, 1H), 8.00 (s, 1H) ³¹P NMR (D₂O): δ 50.53. ¹³C (D₂O): δ 22.53 (d, J=89.68 Hz), 23.55, 54.16, 72.92 (d, J=107.11 Hz), 128.22, 129.13, 129.38, 130.10, 132.43, 138.77, 170.82, 172.49.

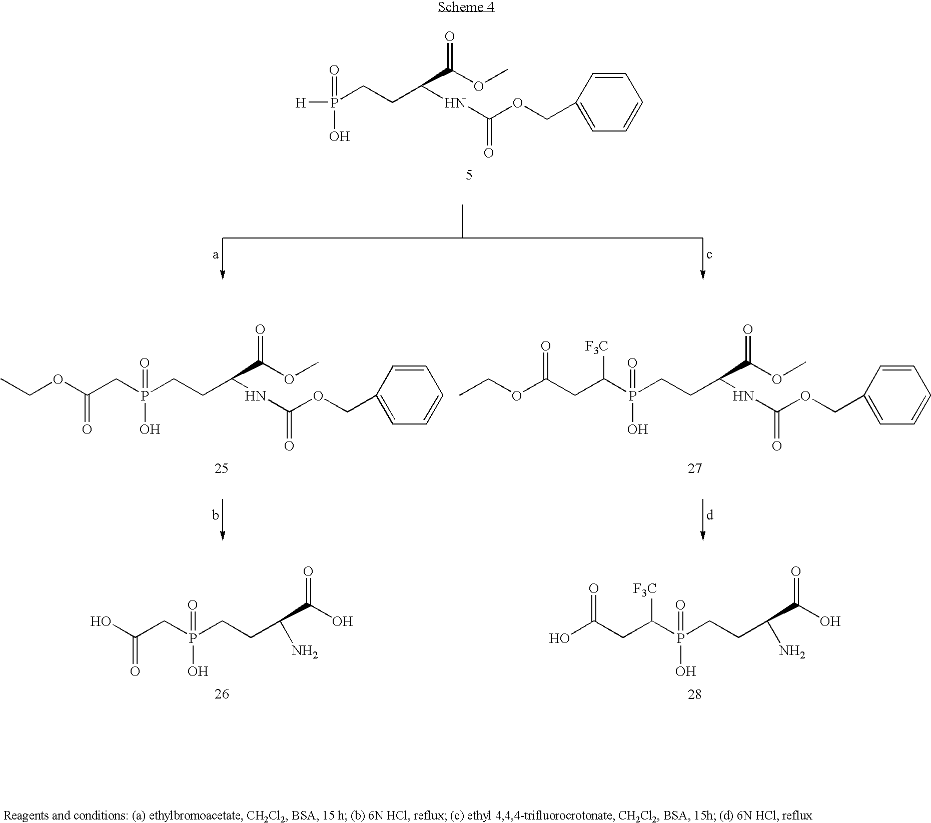

(3S)-2-[((3-(N-Benzyloxycarbonyl)amino-3-methoxycarbonyl)propyl)(hydroxy)phosphinyl] ethanoic Acid Ethyl Ester (25). The compound was prepared from 5 (0.8 mmol) and ethylbromoacetate (501 mg, 3 mmol) by following the procedure described for preparation of compound 15 (60% yield). ¹H NMR (CD₃OD): δ 1.28 (t, J=7.1 Hz, 3H), 2.07 (m, 4H), 3.00 (d, J=17.3 Hz, 2H), 3.76 (s, 3H), 4.19 (q, J=7.1 Hz, 2H), 4.31 (m, 1H), 5.13 (s, 2H), 7.34 (m, 5H). ¹³C NMR (CD₃OD): δ 13.5, 24.2, 25.6 (d, J=99 Hz), 37.2 (d, J=82 Hz), 51.9, 54.8, 61.6, 66.8, 127.9, 128.1, 128.5, 137.1, 157.6, 167.1, 172.7. ³¹P NMR (CD₃OD): δ 45.7. MS (ESI): m/z 400.1 (M−1).

(3S)-2-[((3-amino-3-carboxy)propyl)(hydroxy)phosphinyl]ethanoic Acid (26). The removal of the protecting groups in compound 25 was accomplished using the same procedure as that used for compound 16 and purified by Dowex AG50×4 column to afford 26 (quantitative yield). ¹H NMR (D₂O): δ 1.86 (m, 2H), 2.22 (m, 2H), 2.82 (d, J=16.9 Hz, 2H), 4.08 (t, J=6.2 Hz, 1H). ³¹P NMR (D₂O): δ 46.61. ¹³C NMR (D₂O): δ 21.85, 21.18 (d, J=96.0 Hz), 36.83 (d, J=76.7 Hz), 51.85, 170.33, 170.70. Mass (ESI): 226.1 (M−1). [α]_{D}+14.8° (c 0.1, H₂O).

(3S)-3-[((3-(N-Benzyloxycarbonyl)amino-3-methoxycarbonyl)propyl)(hydroxy)phosphinyl]-3-trifluoromethylpropanoic Acid Ethyl Ester (27). The compound was prepared from 5 (0.8 mmol) and ethyl 4,4,4-trifluorocrotonate (504 mg, 3 mmol) by following the procedure described for preparation of compound 15 (64% yield). ¹H NMR (CD₃OD): δ 1.24 (m, 3H), 2.02 (m, 4H), 2.87 (m, 3H), 3.74 (s, 3H), 4.17 (q, J=6.8 Hz, 2H), 4.24 (m, 1H), 5.12 (s, 2H), 7.36 (5H). ³¹P NMR (CD₃OD): δ543.24.

(3S)-3-[((3-amino-3-carboxy)propyl)(hydroxy)phosphinyl]-3-trifluoromethylpropanoic Acid (28). The removal of the protecting groups in compound 27 was accomplished using the same procedure as that used for compound 16 to afford 28. Compound 27 was purified by Dowex AG50×4 column (quantitative yield). ¹H NMR (D₂O): δ 1.81 (m, 2H), 2.17 (m, 2H), 2.79 (m, 2H), 3.15 (m, 1H), 4.07 (m, 1H). ³¹P NMR (D₂O): δ 43.93, 46.21.

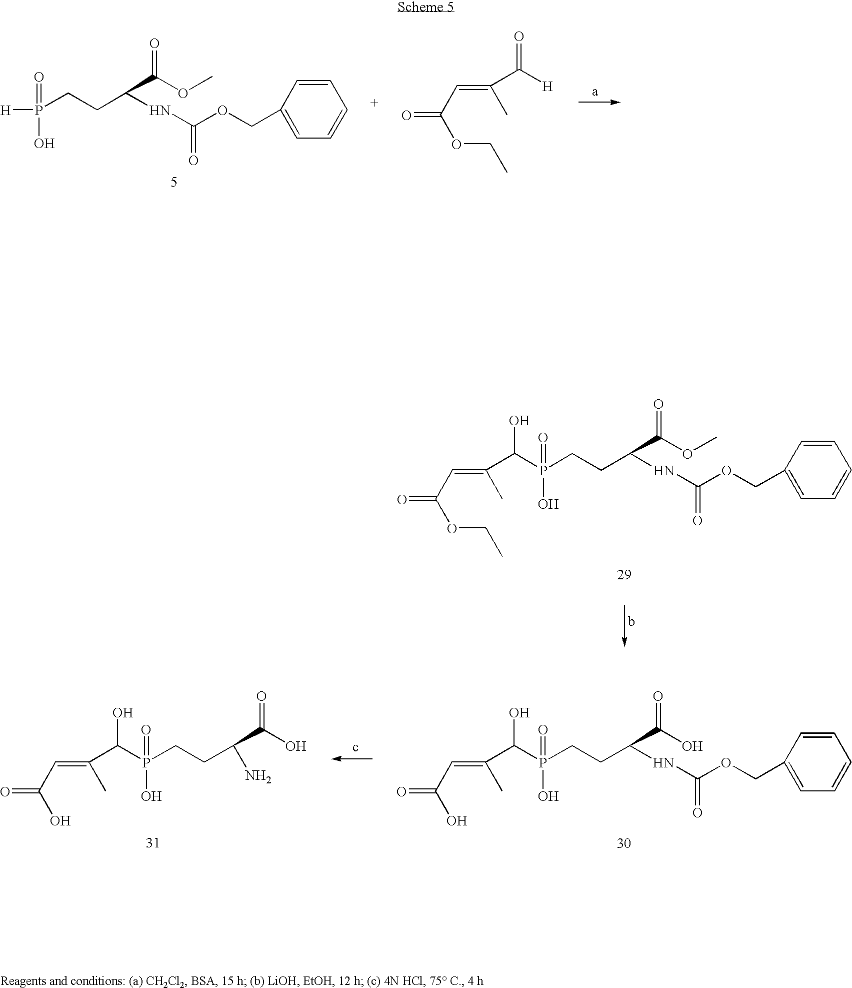

(3S)-4-[((3-(N-Benzyloxycarbonyl)amino-3-methoxycarbonyl)propyl)(hydroxy)phosphinyl]-4-hydroxy-3-methyl-2-butenoic Acid Ethyl Ester (29). The compound was prepared from 5 (0.8 mmol) and ethyl-3-methyl-4-oxocrotonate (426 mg, 3 mmol) by following the procedure described for preparation of compound 15. ¹H NMR (CD₃OD): δ 1.26 (m, 3H), 2.16 (m, 7H), 3.74 (s, 3H), 4.19 (m, 3H), 4.48 (d, J=13.69 Hz, 1H), 5.12 (s, 2H), 5.86 (m, 1H), 7.36 (m, 5H). ³¹P NMR (CD₃OD): δ 49.0. ¹³C NMR (CD₃OD): δ 13.96, 17.65, 22.63 (d, J=89.68 Hz), 24.15, 52.17, 54.76, 60.73, 66.92, 74.05 (d, J=101.52 Hz), 117.03, 127.98, 128.23, 128.68, 137.07, 155.87, 172.09, 172.81.

(3S)-4-[((3-(N-Benzyloxycarbonyl)amino-3-carboxy)propyl)(hydroxy)phosphinyl]-4-hydroxy-3-methyl-2-butenoic Acid (30). Compound 29 (472 mg) was dissolved in 10 ml of ethanol and 10 ml of water. Lithium hydroxide (144 mg, 6 mmol) in 5 ml water was added to the solution, which was stirred at room temperature for 12 h. Following removal of ethanol under vacuo, the resulting solution pH was adjusted to 1. Then extracted with ethyl acetate twice. The organic layer was washed with brine and dried with anhydrous MgSO₄, and the solvent was evaporated in vacuo (66% yield over two steps). ¹H NMR (CD₃OD): δ 1.88 (m, 7H), 4.21 (m, 1H), 4.39 (d, J=12.1 Hz, 1H), 5.11 (s, 2H), 5.93 (m, 1H), 7.39 (m, 5H). ³¹P NMR (CD₃OD): δ 49.4.

(3S)-4-[((3-amino-3-carboxy)propyl)(hydroxy)phosphinyl]-4-hydroxy-3-methyl-2-butenoic Acid (31). The removal of the benzyloxy carbonyl group was accomplished by adding 4N HCl (5 ml) to the compound 30 (140 mg). The resulting solution was stirred at 75° C. for 4 h and cooled to room temperature. Volatile organic byproducts and water were removed under vacuo. The compound 31 was purified by anion exchange AG1×4 column using the procedure described for compound 10. Compound 31 was eluted with 0.3M HCOOH (quantitative yield). ¹H NMR (D₂O): δ 1.86 (m, 2H), 2.16 (m, 5H), 4.10 (m, 1H), 4.43 (d, J=13.34 Hz, 1H), 5.99 (d, J=3.85 Hz, 1H). ¹³C NMR (D₂O): δ 17.2, 22.40 (d, J=100.23 Hz), 23.19, 53.5, 75.25 (d, J=106.91 Hz), 116.35, 157.19, 170.54, 171.79. ³¹P NMR (D₂O): δ 53.14.

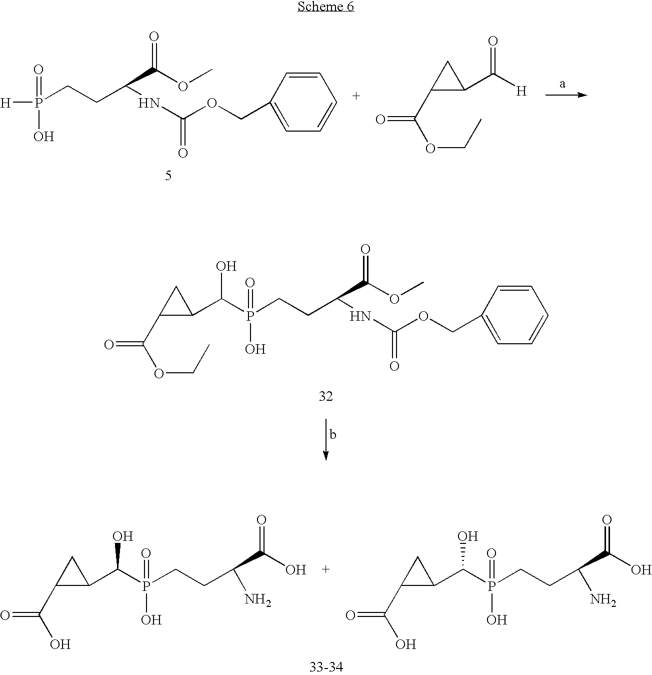

(3S)-2-[(((3-(N-Benzyloxycarbonyl)amino-3-methoxycarbonyl)propyl)(hydroxy)phosphinyl)-hydroxymethyl]cyclopropane-1-carboxylic Acid Ethyl Ester (32). The compound was prepared from 5 (0.8 mmol) and trans ethyl 2-formyl-1-cyclopropanecarboxylate (426 mg, 3 mmol) by following the procedure described for preparation of compound 15 (55% yield). ¹H NMR (CD₃OD): δ 1.19 (m, 5H), 1.96 (m, 6H), 3.40 (m, 0.5H), 3.67 (m, 0.5H), 3.73 (s, 3H), 4.12 (m, 2H), 4.29 (m, 1H), 5.11 (s, 2H), 7.37 (m, 5H). ³¹P NMR (CD₃OD): δ 50.53.

(3S-2-[(((3-amino-3-carboxy)propyl)(hydroxy)phosphinyl)-hydroxymethyl]cyclopropane-1-carboxylic Acid (33). The removal of the protecting groups in compound 27 was accomplished using the same procedure as that used for compound 16 to afford 33 and 34 (1:1, quantitative yield). The mixture (26 mg) of the isomers 33 and 34 was separated by Dowex 50×4 (H⁺, 200-400 mesh, 44×2.2 cm, water elution) chromatography. Diastereoisomers 33 and 34 were separated in fractions 9-14 (4.4 mg) and 33-37 (3.1 mg) respectively. 33: ¹H NMR (D₂O): δ 1.12 (m, 1H), 1.25 (m, 1H), 1.80 (m, 4H), 2.13 (m, 2H), 3.40 (m, 1H), 3.96 (m, 1H). ³¹P NMR (D₂O): δ 51.50. 34: ¹H NMR (D₂O): δ 1.14 (m, 1H); 1.34 (m, 1H); 1.82 (m, 4H); 2.15 (m, 2H); 3.15 (m, 1H); 4.00 (m, 1H). ³¹P NMR (D₂O): δ 51.50. ¹³C NMR (D₂O): δ 13.32 (d, J=78.49 Hz), 18.37 (d, J=42.77 Hz), 22.63 (d, J=79.30 Hz), 23.23 (d, J=78.93 Hz), 23.26, 53.94, 70.56 (d, J=109.05 Hz), 72.31 (d, J=112.07 Hz), 172.47, 178.52.

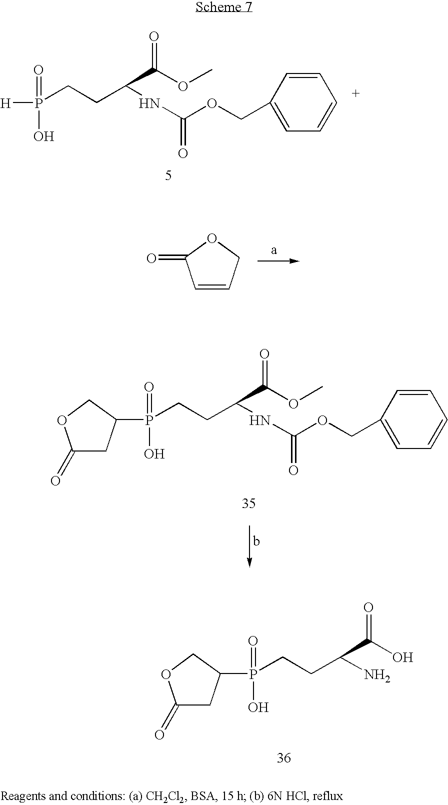

(3S)-4-[((3-(N-Benzyloxycarbonyl)amino-3-methoxycarbonyl)propyl)(hydroxy)phosphinyl]furanone (35). The compound was prepared from 5 (0.8 mmol) and 2-(5H)-furanone (252 mg, 3 mmol) by following the procedure described for preparation of compound 15 (75% yield). ¹H NMR (CD₃OD): δ 1.88 (m, 2H), 2.15 (m, 2H), 2.73 (m, 2H), 3.01 (m, 1H), 3.76 (s, 3H), 4.36 (m, 1H), 4.51 (m, 2H), 5.13 (s, 2H), 7.36 (m, 5H). ³¹P NMR (CD₃OD): δ 49.2. ¹³C NMR (CD₃OD): δ 24.05, 24.69 (d, J=80.81), 28.63, 34.51 (d, J=96.66), 51.93, 54.90, 66.82, 67.71, 127.91, 128.13, 128.55, 137.12, 157.64, 172.76, 177.27 (d, J=9.94).

(3S)-4-[((3-amino-3-carboxy)propyl)(hydroxy)phosphinyl]furanone (36). The removal of the protecting groups in compound 35 was accomplished using the same procedure as that used for compound 16 and purified by Dowex AG50×4 column to afford 36 (quantitative yield). ¹H NMR (D₂O): δ 1.72 (m, 2H), 2.15 (m, 2H), 2.80 (m, 3H), 4.10 (d, J=6.4 Hz, 1H), 4.42 (m, 1H), 4.61 (m, 1H). ³¹P NMR (D₂O): δ 51.21. ¹³C NMR (D₂O): δ 23.19, 24.45 (d, J=93.71 Hz), 29.47, 34.86 (d, J=96.14 Hz), 54.2, 69.44, 172.03, 180.73 (d, J=10.2).

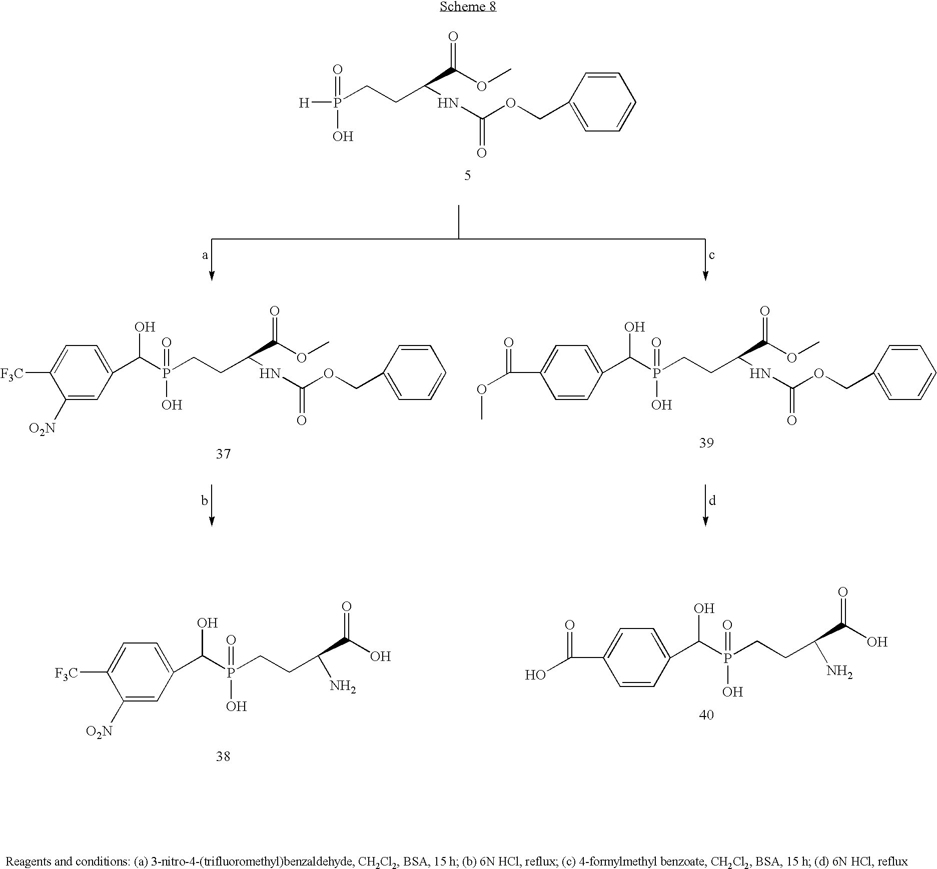

(3S)-3-[(((3-(N-Benzyloxycarbonyl)amino-3-methoxycarbonyl)propyl)(hydroxy)phosphinyl)-hydroxymethyl]-6-trifluoromethyl-1-nitrobenzene (37). 37 was prepared from 5 (0.8 mmol) and 3-nitro-4-(trifluoromethyl)benzaldehyde (657 mg, 3 mmol) by following the procedure described for preparation of compound 15. ¹H NMR (CD₃OD): δ 2.04 (m, 4H), 3.75 (s, 3H), 4.33 (m, 1H), 5.15 (s, 2H), 6.21 (m, 1H), 7.36 (m, 5H), 8.15 (m, 3H). ³¹P NMR (CD₃OD): δ 48.14.

(3S)-3-[(((3-amino-3-carboxy)propyl)(hydroxy)phosphinyl)-6-trifluoromethyl-1-nitrobenzene (38). The removal of the protecting groups in compound 37 was accomplished using the same procedure as that used compound 16 and purified by Dowex AG50×4 column to afford 38. ¹H NMR (D₂O): δ 1.75 (m, 2H), 2.10 (m, 2H), 4.01 (m, 1H), 5.97 (d, J=10.95 Hz, 1H), 8.02 (m, 2H); 8.35 (s, 1H). ³¹P NMR (D₂O): δ 48.25.

(3S)-4-[(((3-(N-Benzyloxycarbonyl)amino-3-methoxycarbonyl)propyl)(hydroxy)phosphinyl)-hydroxymethyl]benzoic Acid Methyl Ester (39). 39 was prepared from 5 (0.8 mmol) and methyl 4-formylbenzoate (492 mg, 3 mmol) by using the procedure described for preparation of compound 15. ¹H NMR (CD₃OD): δ 2.03 (m, 4H), 3.73 (s, 3H), 3.92 (s, 3H), 4.29 (m, 1H), 5.21 (s, 2H), 5.47 (m, 1H), 7.37 (m, 5H), 7.65 (m, 2H), 8.03 (m, 2H). ³¹P NMR (D₂O): δ 48.81. ¹³C NMR (CD₃OD): δ 23.39 (d, J=91.57 Hz), 24.12, 51.82, 66.88, 72.16 (d, J=108.61 Hz), 127.41, 127.86, 128.13, 128.60, 129.34, 129.66, 137.06, 143.48, 157.65, 167.53, 172.78.

(3S)-4-[(((3-amino-3-carboxy)propyl)(hydroxy)phosphinyl)-hydroxymethyl]benzoic Acid (40). The removal of the protecting groups in compound 39 was accomplished following the same procedure as that followed for compound 16 and purified by Dowex AG50×4 column to afford 40. ¹H NMR (D₂O): δ 1.65 (m, 2H), 1.94 (m, 2H), 3.94 (m, 1H), 4.90 (d, J=10.4 Hz, 1H), 7.48 (d, J=7.19 Hz, 2H), 7.93 (d, J=8.22 Hz, 2H). ³¹P NMR (D₂O): δ 49.89. ¹³C NMR (D₂O): δ 22.71 (d, J=90.3 Hz), 23.70, 54.52, 73.44 (d, J=104.96 Hz), 127.34, 129.21, 130.00, 144.38, 170.89, 172.73.

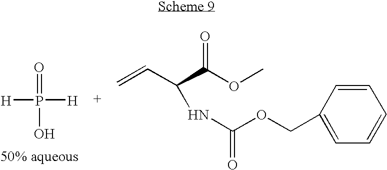

(2S)-2-(N-Benzyloxycarbonyl)amino-4-[(hydroxy)phosphinyl]butanoic Acid Methyl Ester (5). A mixture of hypophosphorous acid (660 mg, 5 mmol, 50% aqueous), Z-L-vinyl glycine methyl ester (250 mg, 1 mmol) and α,α′-azoisobutyronitrile (AIBN, 8.1 mg, 0.05 mmol) in methanol (1 ml) was refluxed at 80° C. for 5 h. Then the methanol was evaporated under vacuum and the residue was extracted with ethyl acetate, dried over MgSO₄. The organic layer was evaporated under vacuum and purified by Silica gel chromatography (CH₂Cl₂:MeOH, 1:0 to 9:1) to afford 5 (90% yield); ¹H NMR (CD₃OD): δ 1.98 (m, 4H), 3.72 (s, 3H), 4.11 (m, 1H), 5.12 (s, 2H), 7.34 (m, 5H). ¹³C NMR (CD₃OD): δ 13.8, 23.4, 26.1 (d, J=92 Hz), 52.2, 54.7, 66.9, 128.0, 128.3, 128.7, 137.2, 157.5, 172.7. ³¹P NMR (CD₃OD): δ 35.3.

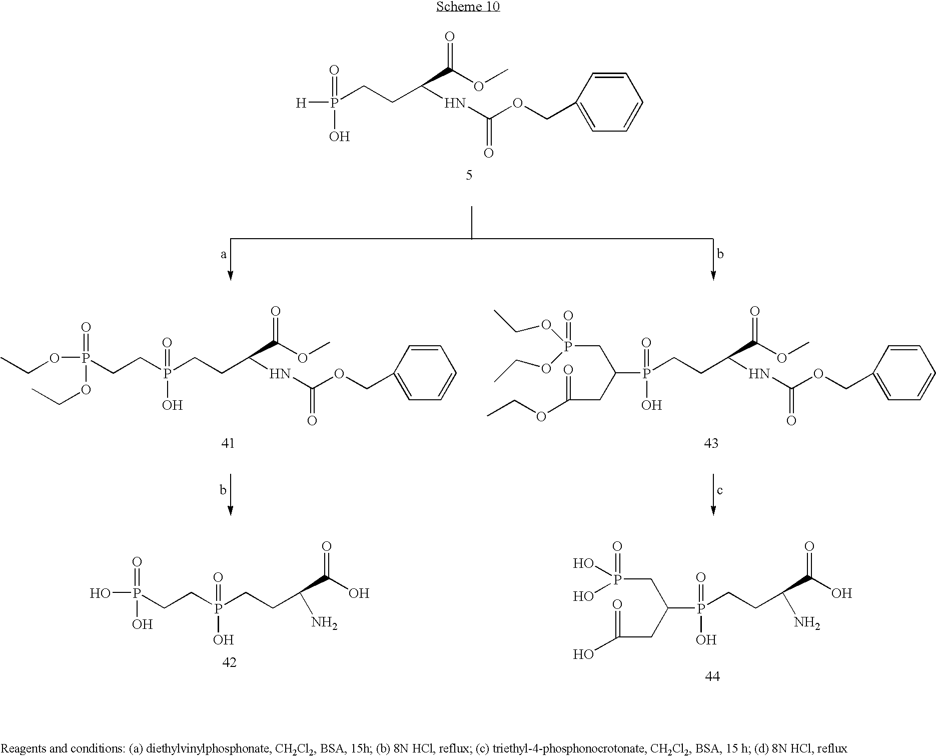

(3S)-2-[((3-(N-Benzyloxycarbonyl)amino-3-methoxycarbonyl)propyl)(hydroxy)phosphinyl] ethylphosphonate Diethyl Ester (41). The compound was prepared from 5 (0.8 mmol) and diethylvinylphosphonate (492 mg, 3 mmol) by using the procedure described for preparation of compound 15 (87.8% yield). ¹H NMR (CD₃OD): δ 1.29 (m, 6H), 1.99 (m, 8H), 3.73 (s, 3H), 4.14 (m, 4H), 4.31 (m, 1H), 5.12 (s, 2H), 7.37 (m, 5H). ³¹P NMR (CD₃OD): δ 32.57 (d, J=65.87 Hz), 52.31 (d, J=65.59 Hz). MS (ESI): m/z 480.1 (M+1).

(3S)-3-[((3-Amino-3-carboxy)propyl)(hydroxy)phosphinyl]ethylphosphonate (42). The removal of the protecting groups in compound 21 was accomplished following the same procedure as that followed for compound 16 and purified by anion exchange AG1×4 column using the procedure as described for compound 10. The compound 42 was eluted with 0.8-1.0M HCOOH (quantitative yield). ¹H NMR (D₂O): δ 1.75 (m, 6H), 2.00 (m, 2H), 3.97 (t, J=5.79 Hz, 1H). ³¹P NMR (D₂O): δ 38.21 (d, J=65.00 Hz), 61.99 (d, J=65.13 Hz). MS (ESI): m/z 276.1 (M+1).

(3S)-2-[(((3-(N-Benzyloxycarbonyl)amino-3-methoxycarbonyl)propyl)(hydroxy)phosphinyl)-methyl]-2-(diethylphosphonomethy)-propanoic Acid Ethyl Ester (43). The compound was prepared from 5 (0.8 mmol) and triethyl-4-phosphonocrotonate (750 mg, 3 mmol) by a procedure similar to that for the preparation of compound 15 (83.8% yield). ¹H NMR (CD₃OD): δ 1.28 (m, 9H), 2.19 (m, 6H), 2.81 (m, 3H), 3.73 (s, 3H), 4.12 (m, 6H), 4.32 (m, 1H), 5.12 (s, 2H), 7.37 (m, 5H). ³¹P NMR (CD₃OD): δ 31.29 (d, J=57.26 Hz), 53.97 (d, J=57.27 Hz). MS (ESI): m/z 566.1 (M+1).

(3S)-2-[(((3-(N-Benzyloxycarbonyl)amino-3-methoxycarbonyl)propyl)(hydroxy)phosphinyl)-methyl]-2-(phosphonomethy)-propanoic Acid (44). The removal of the protecting groups in compound 43 was accomplished using the same procedure as that used compound 16 and purified by anion exchange AG1×4 column using the procedure as described for compound 10. The compound 44 was eluted with 1.0-1.3M HCOOH (quantitative yield). ¹H NMR (D₂O): δ 1.62 (m, 3H), 1.86 (m, 3H), 2.26 (m, 1H), 2.41 (m, 1H), 2.47 (m, 1H), 3.85 (m, 1H). ³¹P NMR (D₂O): δ 37.51 (d, J=50.72 Hz), 63.67 (d, J=50.67 Hz). ¹³C NMR (CD₃OD): δ 22.20 (d, J=91.35 Hz), 22.47, 25.11 (d, J=136.71 Hz), 29.72 (d, J=92.69 Hz), 33.38, 52.98, 171.12, 175.30 (d, J=8.05 Hz).

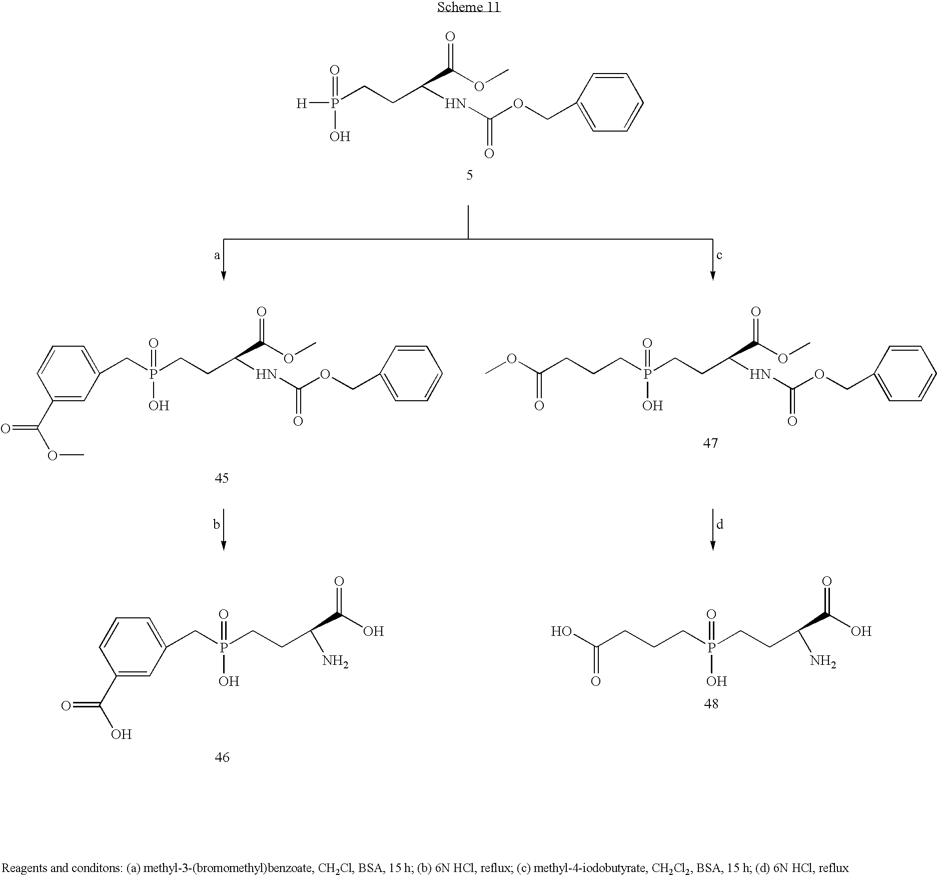

(3S)-3-[(((3-(N-Benzyloxycarbonyl)amino-3-methoxycarbonyl)propyl)(hydroxy)phosphinyl)-methyl]benzoic Acid Methyl Ester (45). The compound was prepared from 5 (0.8 mmol) and methyl-3-(bromomethyl)-benzoate (687 mg, 3 mmol) by using the procedure described for preparation of compound 15 (72.2% yield). ¹H NMR (CD₃OD): δ 1.98 (m, 4H), 3.23 (d, J=15.04 Hz, 2H), 3.69 (s, 3H), 3.86 (s, 3H), 4.31 (m, 1H), 5.09 (s, 2H), 7.33 (m, 5H), 7.43 (m, 2H), 7.94 (m, 2H). ³¹P NMR (CD₃OD): δ 50.20. ¹³C NMR (D₂O): δ 24.35, 24.62 (d, J=93.22 Hz), 36.18, (d, J=88.71 Hz), 52.00, 54.93, 66.91, 128.02, 128.24, 128.68, 128.95, 130.63, 131.21, 133.19 (d, J=7.48 Hz), 134.93, 137.11, 157.50, 172.75, 174.39. MS (ESI): m/z 464.1 (M+1).

(3S)-3-[(((3-amino-3-carboxy)propyl)(hydroxy)phosphinyl)-methyl]benzoic Acid (46). The removal of the protecting groups in compound 45 was accomplished following the same procedure as that followed for compound 16 and purified by Dowex AG50×4 column to afford 46 (quantitative yield). ¹H NMR (D₂O): δ 1.60 (m, 2H, 2.00 (m, 2H), 3.08 (d, J=16.78 Hz, 2H), 3.93 (t, J=6.02 Hz, 1H), 7.39 (m, 2H), 7.76 (m, 2H). ³¹P NMR (D₂O): δ 54.66. ¹³C NMR (D₂O): δ 23.42, 24.38 (d, J=91.69 Hz), 37.21 (d, J=85.97 Hz), 53.80 (d, J=14.83 Hz), 128.24, 129.34, 130.24, 130.86, 133.98 (d, J=67.61 Hz), 135.11, 170.68, 172.20. MS (ESI): m/z 302.1 (M+1).

(3S)-4-[((3-(N-Benzyloxycarbonyl)amino-3-methoxycarbonyl)propyl)(hydroxy)phosphinyl]butanoic Acid Methyl Ester (47). The compound was prepared from 5 (0.8 mmol) and methyl-4-iodobutyrate (684 mg, 3 mmol) by using the procedure described for preparation of compound 15 (64.6% yield). ¹H NMR (CD₃OD): δ 2.14 (m, 10H), 3.68 (s, 3H), 3.74 (s, 3H), 4.34 (m, 1H), 5.12 (s, 2H), 7.36 (m, 5H). ³¹P NMR (CD₃OD): δ 55.43. MS (ESI): m/z 416.1 (M+1).

(3S)-4-[((3-Amino-3-carboxy)propyl)(hydroxy)phosphinyl]butanoic Acid (48). The removal of the protecting groups in compound 47 was accomplished following the same procedure as that followed for compound 16 and purified by Dowex AG50×4 column to afford 48 (quantitative yield). ¹H NMR (D₂O): δ 1.71 (m, 6H), 2.08 (m, 2H), 2.44 (m, 2H), 3.93 (t, J=5.98 Hz, 1H). ³¹P NMR (D₂O): δ 58.80. MS ESI): m/z 254.1 (M+1).

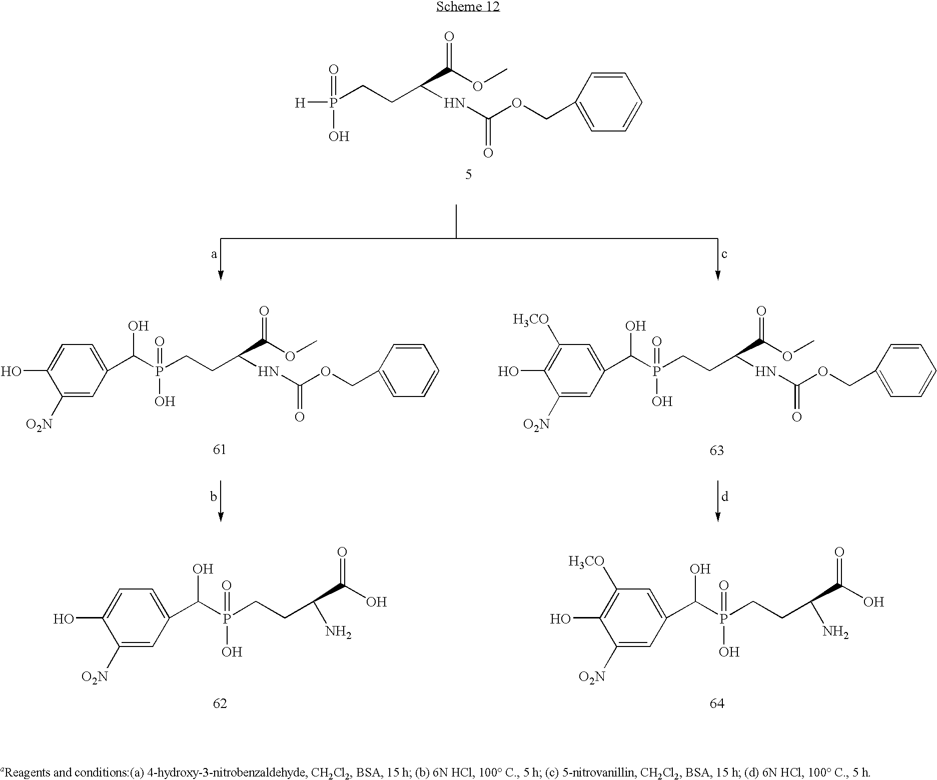

(3S)-3-[(((3-(N-Benzyloxycarbonyl)amino-3-methoxycarbonyl)propyl)(hydroxy) phosphinyl)-hydroxymethyl]-6-hydroxy-1-nitrobenzene (61). 61 was prepared from 5 (0.8 mmol) and 4-hydroxy-3-nitrobenzaldehyde (401 mg, 2.4 mmol) by following the procedure described for preparation of compound 15. ¹H NMR (CD₃OD): δ 2.02 (m, 4H), 3.74 (s, 3H), 4.33 (m, 1H), 5.02 (d, J=8.60 Hz, 1H), 5.09 (s, 2H), 7.13 (d, J=8.54 Hz, 1H), 7.31 (m, 5H), 7.61 (d, J=8.76 Hz, 1H), 8.08 (s, 1H). ³¹P NMR (CD₃OD): δ 48.76.

(3S)-3-[(((3-amino-3-carboxy)propyl)(hydroxy)phosphinyl)-hydroxymethyl]-6-hydroxy-1-nitrobenzene (62). The removal of the protecting groups in compound 61 was accomplished using the same procedure as that used compound 16 and purified by Dowex AG50×4 column to afford 62. ¹H NMR (D₂O): δ 1.78 (m, 2H), 2.05 (m, 2H), 3.98 (m, 1H), 4.80 (d, J=8.56 Hz, 1H), 7.06 (d, J=8.66 Hz, 1H); 7.57 (d, J=8.63 Hz, 1H), 8.02 (s, 1H). ¹³C NMR (D₂O): δ 22.59 (d, J=88.4 Hz), 23.61, 53.98 (d, J=14.72 Hz), 71.75 (d, J=107.37 Hz), 119.99, 123.70, 130.83, 134.31, 136.83, 153.28, 172.36.

(3S)-3-[(((3-(N-Benzyloxycarbonyl)amino-3-methoxycarbonyl)propyl)(hydroxy) phosphinyl)-hydroxymethyl]-5-methoxy-6-hydroxy-1-nitrobenzene (63). 63 was prepared from 5 (0.8 mmol) and 5-nitrovanillin (473 mg, 2.4 mmol) by using the procedure described for preparation of compound 15. ¹H NMR (CD₃OD): δ 2.12 (m, 4H), 3.73 (s, 3H), 3.92 (s, 3H), 4.32 (m, 1H), 5.08 (s, 2H), 5.33 (m, 1H), 7.53 (m, 7H). ³¹P NMR (CD₃OD): δ 49.29.

(3S)-3-[(((3-amino-3-carboxy)propyl)(hydroxy)phosphinyl)-hydroxymethyl]-5-methoxy-6-hydroxy-1-nitrobenzene (64). The removal of the protecting groups in compound 63 was accomplished following the same procedure as that followed for compound 16 and purified by Dowex AG50×4 column to afford 64. ¹H NMR (D₂O): δ 1.74 (m, 2H), 2.03 (m, 2H), 3.83 (s, 3H), 4.02 (m, 1H), 7.23 (s, 1H), 7.59 (s, 1H). ³¹P NMR (D₂O): δ 50.03. ¹³C NMR (D₂O): δ 22.63 (d, J=90.45 Hz), 23.68, 54.16 (d, J=12.65 Hz), 56.91, 72.10 (d, J=109.9 Hz), 114.43, 116.81, 129.99, 134.24, 143.91, 149.30, 172.56. MS (ESI) m/z: 365.1 (M+1).

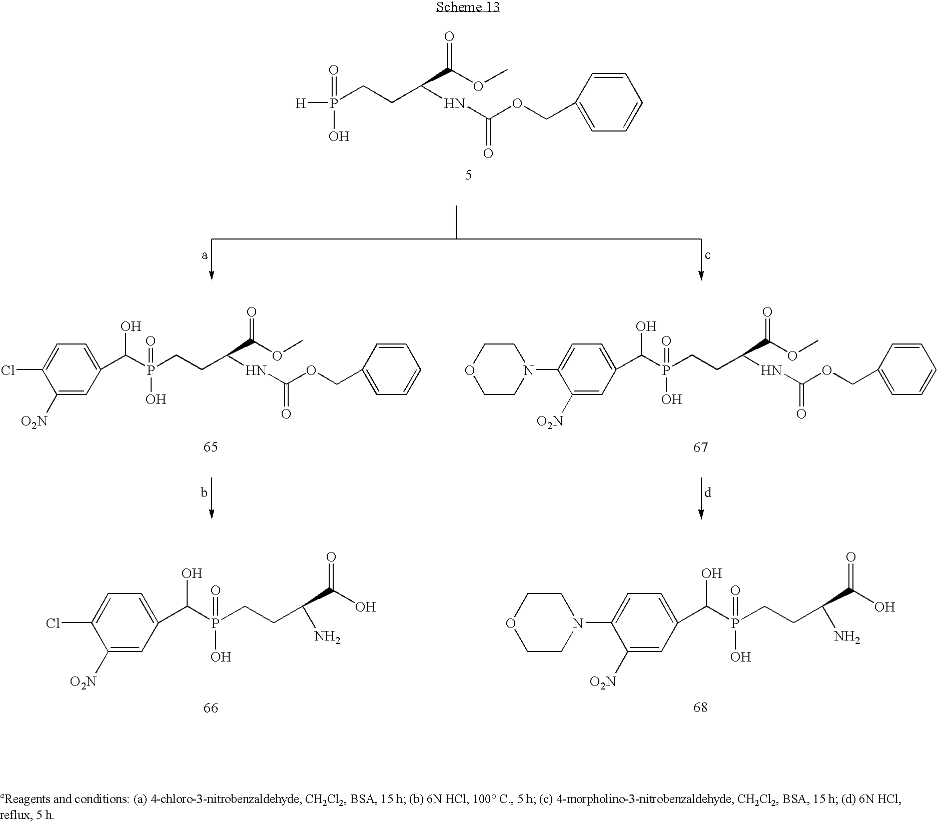

(3S)-3-[(((3-(N-Benzyloxycarbonyl)amino-3-methoxycarbonyl)propyl)(hydroxy) phosphinyl)-hydroxymethyl]-6-chloro-1-nitrobenzene (65). 65 was prepared from 5 (0.8 mmol) and 4-chloro-3-nitrobenzaldehyde (445 mg, 2.4 mmol) by following the procedure described for preparation of compound 15. ¹H NMR (CD₃OD): δ 2.03 (m, 4H), 3.73 (s, 3H), 4.31 (m, 1H), 5.11 (s, 2H), 5.25 (d, J=7.0 Hz, 1H), 7.34 (m, 5H), 7.65 (m, 2H), 8.07 (s, 1H). ³¹P NMR (CD₃OD): δ 46.26.

(3S)-3-[(((3-amino-3-carboxy)propyl)(hydroxy)phosphinyl)-hydroxymethyl]-6-chloro-1-nitrobenzene (66). The removal of the protecting groups in compound 65 was accomplished using the same procedure as that used compound 16 and purified by Dowex AG50×4 column to afford 66. ¹H NMR (D₂O): δ 1.73 (m, 2H), 2.12 (m, 2H), 4.02 (m, 1H), 4.91 (d, J=9.91 Hz, 1H), 7.60 (m, 2H); 7.98 (s, 1H). ³¹P NMR (D₂O): δ 49.02. ¹³C NMR (D₂O): δ 22.66 (d, J=87.3 Hz), 23.55, 53.92 (d, J=14.03 Hz), 72.00 (d, J=106.4 Hz), 124.30, 125.79, 132.12, 132.56, 139.37, 147.39, 172.27. MS (ESI) m/z: 353.1 (M+1).

(3S)-3-[(((3-(N-Benzyloxycarbonyl)amino-3-methoxycarbonyl)propyl)(hydroxy) phosphinyl)-hydroxymethyl]-6-morpholino-1-nitrobenzene (67). 67 was prepared from 5 (0.8 mmol) and 4-morpholino-3-nitrobenzaldehyde (566 mg, 2.4 mmol) by using the procedure described for preparation of compound 15. ¹H NMR (CD₃OD): δ 2.05 (m, 4H), 3.07 (m, 4H), 3.74 (s, 3H), 3.80 (m, 4H), 4.27 (m, 1H), 4.96 (d, J=9.18 Hz, 1H), 5.12 (s, 2H), 7.30 (m, 6H), 7.67 (d, J=8.49 Hz, 1H), 7.91 (s, 1H). ³¹P NMR (CD₃OD): δ 45.98.

(3S)-3-[(((3-amino-3-carboxy)propyl)(hydroxy)phosphinyl)-hydroxymethyl]-6-morpholino-1-nitro benzene (68). The removal of the protecting groups in compound 67 was accomplished following the same procedure as that followed for compound 16 and purified by Dowex AG50×4 column to afford 68. ¹H NMR (D₂O): δ 1.64 (m, 2H), 2.05 (m, 2H), 3.03 (m, 4H), 3.73 (m, 1H), 3.81 (m, 4H), 4.79 (d, J=8.81 Hz, 1H), 7.27 (d, J=8.54 Hz, 1H), 7.61 (d, J=8.36 Hz, 1H), 7.91 (s, 1H). ³¹P NMR (D₂O): δ 48.06. ¹³C NMR (D₂O): δ 23.13 (d, J=90.9 Hz), 24.04, 52.06, 55.72, 66.88, 72.20 (d, J=106.9 Hz), 121.29, 125.03, 133.60, 133.71, 142.22, 145.25, 174.46.

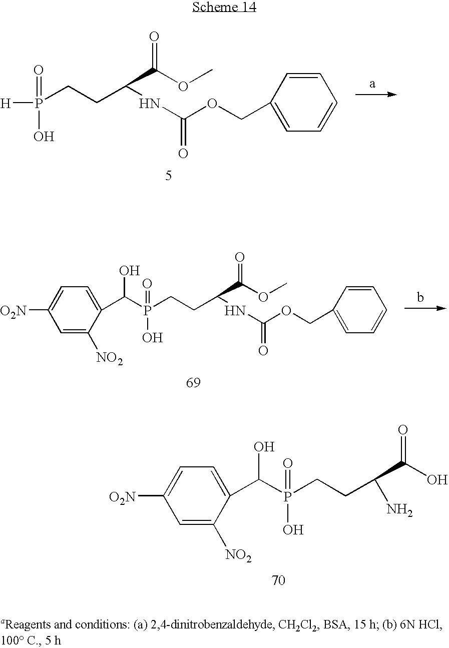

(3S)-4-[(((3-(N-Benzyloxycarbonyl)amino-3-methoxycarbonyl)propyl)(hydroxy) phosphinyl)-hydroxymethyl]-1,3-dinitrobenzene (69). The compound (69) was prepared from 5 (0.8 mmol) and 4-methoxy-3-nitrobenzaldehyde (470 mg, 2.4 mmol) by following the procedure described for preparation of compound 15. ¹H NMR (CD₃OD): δ 2.02 (m, 4H), 3.74 (s, 3H), 4.32 (m, 1H), 5.10 (s, 2H), 6.18 (m, 1H), 7.33 (m, 5H), 8.41 (m, 2H), 8.76 (d, J=8.39 Hz, 1H). ³¹P NMR (CD₃OD): δ 47.96.

(3S)-4-[(((3-amino-3-carboy)propyl)(hydroxy)phosphinyl)-hydroxymethyl]-1,3-dinitrobenzene (70). The removal of the protecting groups in compound 69 was accomplished following the same procedure as that followed for compound 16 and purified by Dowex AG50×4 column to afford 70. ¹H NMR (D₂O): δ 1.80 (m, 2H), 2.04 (m, 2H), 4.02 (m, 1H), 6.02 (d, J=11.15 Hz, 1H), 7.93 (d, J=8.70 Hz, 1H), 8.32 (s, 1H), 8.65 (d, J=7.33 Hz, 1H). ³¹P NMR (D₂O): δ 54.37.

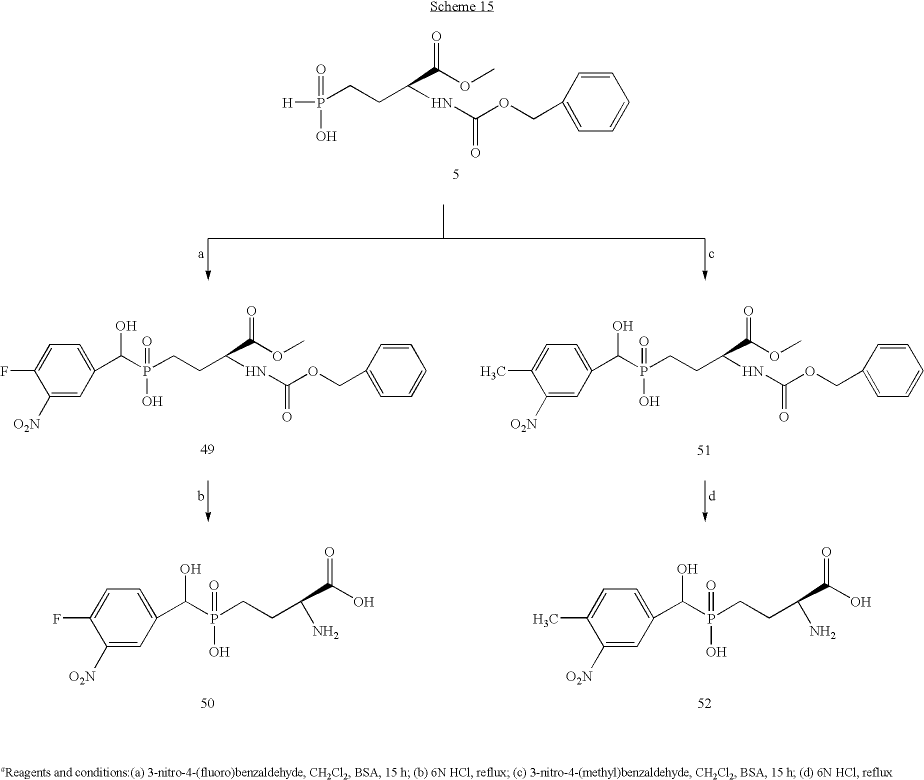

(3S)-3-[(((3-(N-Benzyloxycarbonyl)amino-3-methoxycarbonyl)propyl)(hydroxy)phosphinyl)-hydroxymethyl]-6-fluoro-1-nitrobenzene (49).

49 was prepared from 5 (0.8 mmol) and 3-nitro-4-(fluoro)benzaldehyde (507.3 mg, 3 mmol) by following the procedure described for preparation of compound 15. ¹H NMR (CD₃OD): δ 2.16 (m, 4H), 3.75 (s, 3H), 4.34 (m, 1H), 5.12 (s, 2H), 5.49 (d, J=7.11 Hz, 1H), 7.39 (m, 6H), 7.86 (m, 1H), 8.26 (m, 1H). ³¹P NMR (CD₃OD): δ 47.99.

(3S)-3-[(((3-amino-3-carboxy)propyl)(hydroxy)phosphinyl)-6-fluoro-5-nitrobenzene (50). The removal of the protecting groups in compound 49 was accomplished using the same procedure as that used compound 16 and purified by Dowex AG50×4 column to afford 50. ¹H NMR (D₂O): δ 1.75 (m, 2H), 2.11 (m, 2H), 4.00 (m, 1H), 4.89 (d, J=9.02 Hz, 1H), 7.35 (m, 1H), 7.70 (m, 1H), 8.09 (d, J=6.84 Hz, 1H). ³¹P NMR ([20): δ 49.37.

(3S)-3-[(((3-(N-Benzyloxycarbonyl)amino-3-methoxycarbonyl)propyl)(hydroxy)phosphinyl)-hydroxymethyl]-6-methyl-1-nitrobenzene (51). The compound was prepared from 5 (0.8 mmol) and 3-nitro-4-(methyl)benzaldehyde (495.5 mg, 3 mmol) by using the procedure described for preparation of compound 15. ¹H NMR (CD₃OD): δ 2.14 (m, 4H), 2.55 (s, 3H), 3.74 (s, 3H), 4.32 (m, 1H), 5.12 (s, 2H), 5.46 (d, J=7.54 Hz, 1H), 7.37 (m, 6H), 7.70 (d, J=7.75 Hz, 1H), 8.13 (s, 1H). ³¹P NMR (CD₃OD): δ 48.69.

(3S)-3-[(((3-amino-3-carboxy)propyl)(hydroxy)phosphinyl)-6-methyl-1-nitrobenzene (52). The removal of the protecting groups in compound 51 was accomplished using the same procedure as that used compound 16 and purified by Dowex AG50×4 column to afford 52. ¹H NMR (D₂O): δ 1.61 (m, 2H), 2.03 (m, 2H), 2.47 (s, 3H), 3.68 (m, 1H), 4.81 (d, J=8.89 Hz, 1H), 7.36 (d, J=7.92 Hz, 1H), 7.54 (d, J=7.78 Hz, 1H), 7.96 (s, 1H). ³¹P NMR (D₂O): δ 47.99.

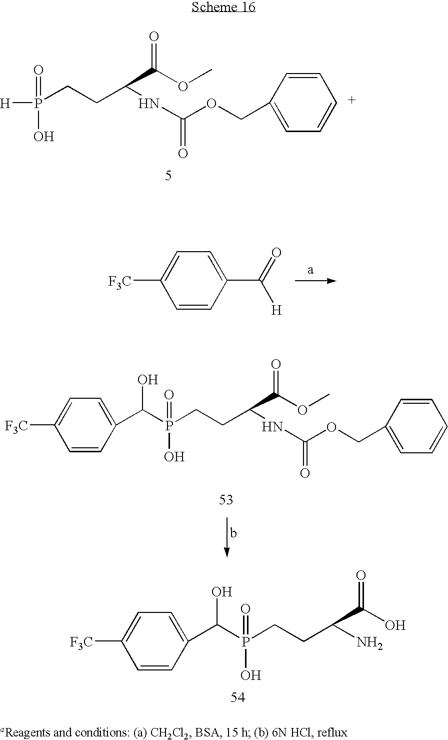

(3S)-4-[(((3-(N-Benzyloxycarbonyl)amino-3-methoxycarbonyl)propyl)(hydroxy)phosphinyl)-hydroxymethyl]-1-trifluoromethylbenzene (53). The compound was prepared from 5 (0.8 mmol) and 4-(trifluoromethyl)benzaldehyde (522.4 mg, 3 mmol) by a procedure similar to that for the preparation of compound 15. ¹H NMR (CD₃OD): δ 2.13 (m, 4H), 3.72 (s, 3H), 4.32 (m, 1H), 5.11 (m, 3H), 7.35 (m, 5H), 7.72 (m, 4H). ³¹P NMR (CD₃OD): δ 48.75.

(3S)-4-[(((3-amino-3-carboxy)propyl)hydroxy)phosphinyl)-1-trifluoromethylbenzene (54). The removal of the protecting groups in compound 53 was accomplished using the same procedure as that used compound 16 and purified by Dowex AG50×4 column to afford 54. ¹H NMR (D₂O): δ 1.65 (m, 2H), 1.94 (m, 2H), 3.94 (m, 1H), 4.89 (d, J=9.99 Hz, 1H), 7.50 (d, A 7.94 Hz, 2H), 7.64 (d, J=7.93 Hz, 2H). ³¹P NMR (D₂O): δ 50.59.

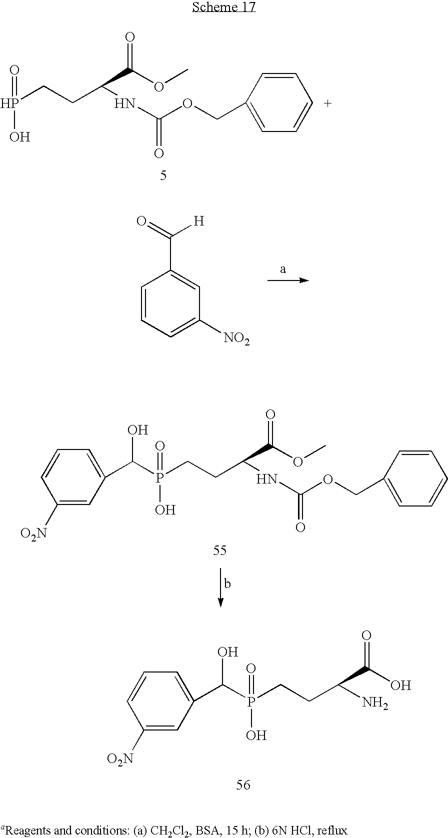

(3S)-3-[(((3-(N-Benzyloxycarbonyl)amino-3-methoxycarbonyl)propyl) (hydroxy) phosphinyl)-hydroxymethyl]3-nitrobenzene (55). The compound was prepared from 5 (0.8 mmol) and 3-nitrobenzaldehyde (453 mg, 3 mmol) by using the procedure described for preparation of compound 15. ¹H NMR (CD₃OD): δ 2.15 (m, 4H), 3.73 (s, 3H), 4.31 (m, 1H), 5.12 (s, 2H), 5.16 (m, 1H), 7.34 (m, 5H), 7.61 (m, 1H), 7.91 (m, 1H), 8.16 (m, 1H), 8.42 (s, 1H). ³¹P NMR (CD₃OD): δ 48.40.

(3S)-3-[(((3-amino-3-carboxy)propyl)(hydroxy)phosphinyl)-hydroxymethyl] 3-nitrobenzene (56). The removal of the protecting groups in compound 55 was accomplished following the same procedure as that followed for compound 16 and purified by Dowex AG50×4 column to afford 56 (quantitative yield). ¹H NMR (D₂O): δ 1.72 (m, 2H), 2.09 (m, 2H), 4.01 (m, 1H), 4.94 (d, J=9.57 Hz, 1H), 7.53 (t, J=67.99 Hz, 1H), 7.74 (d, J=7.53 Hz, 1H), 8.09 (d, J=8.17 Hz, 1H), 8.20 (s, 1H). ³¹P NMR (D₂O): δ 49.69.

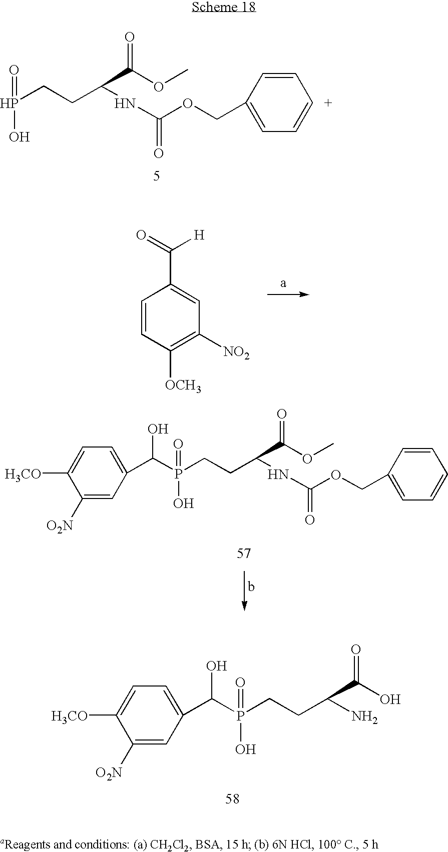

(3S)-3-[(((3-(N-Benzyloxycarbonyl)amino-3-methoxycarbonyl)propyl) (hydroxy) phosphinyl)-hydroxymethyl] 3-nitro-4-methoxybenzene (57). The compound was prepared from 5 (0.8 mmol) and 4-methoxy-3-nitrobenzaldehyde (543 mg, 3 mmol) by following the procedure described for preparation of compound 15. ¹H NMR (CD₃OD): δ 2.10 (m, 4H), 3.73 (s, 3H), 3.92 (s, 3H), 4.31 (m, 1H), 5.02 (d, J=8.49 Hz, 1H), 5.12 (s, 2H), 7.22 (d, J=8.63 Hz, 1H), 7.35 (m, 5H), 7.72 (d, J=8.3 Hz, 1H), 8.01 (s, 1H). ³¹P NMR (CD₃OD): δ 48.73.

(3S)-3-[(((3-amino-3-carboxy)propyl)(hydroxy)phosphinyl)-hydroxymethyl] 3-nitro-4-methoxybenzene (58). The removal of the protecting groups in compound 57 was accomplished following the same procedure as that followed for compound 16 and purified by Dowex AG50×4 column to afford 58. ¹H NMR (D₂O): δ 1.73 (m, 2H), 2.09 (m, 2H), 3.91 (s, 3H), 4.00 (m, 1H), 4.83 (d, J=8.67 Hz, 1H), 7.24 (d, J=8.8 Hz, 1H), 7.64 (d, J=8.78 Hz, 1H), 7.93 (s, 1H). ³¹P NMR (D₂O): δ 50.13.

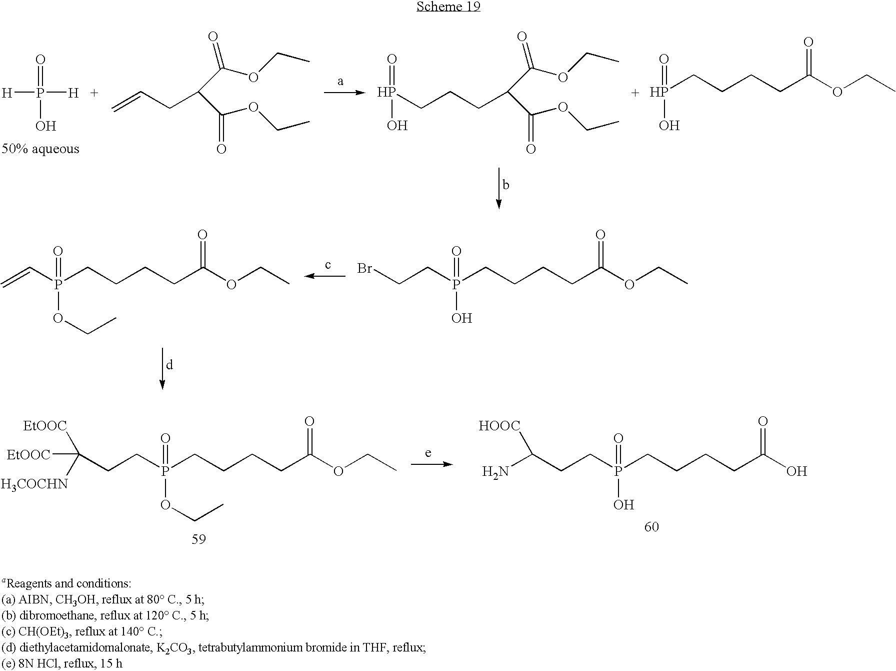

5-[((3-(N-Acetyl)amino)-3-(bisethoxycarbonyl)propyl)(ethoxy)phosphinyl]pentanoic Acid Ethyl Ester (59). A mixture of hypophosphorous acid (3.3 g, 25 mmol, 50% aqueous), diethylallylmalonate (1 mg, 5 mmol) and α,α′-azoisobutyronitrile (AIBN, 41 mg, 0.25 mmol) in methanol (2 ml) was refluxed at 80° C. for 5 h. Then the methanol was evaporated under vacuum and the residue was extracted with ethyl acetate, dried over MgSO₄. The organic layer was evaporated under vacuum. Then the crude product (1.338 g) was mixed with dibromoethane (2.4 ml, 28 mmol) and hexamethydisilazane (2.96 ml, 14 mmol) was heated at 120° C. for 9 h. The formed trimethylbromosilane and excess dibromoethane were removed under vacuum. Then 50 ml of aqueous ethanol (1:1) were added dropwise to the residue and refluxed for 0.5 h. Then the solvent was removed under vacuum and extracted with ethyl acetate. The organic layer was dried over MgSO₄ and the solvent was removed under vacuum. The crude product (270 mg) was treated with 40 ml of triethyl orthoformate, and the mixture was refluxed with a Dean-Stark trap to remove ethanol and ethyl formate. Excess of triethylorthoformate was removed under vacuum. The crude product (200 mg) was mixed with diethylacetamidomalonate (174 mg, 0.8 mmol), potassium carbonate (221 mg, 1.6 mmol) and tetrabutylammonium bromide (13 mg, 0.04 mmol) in THF (1 ml). The reaction mixture was refluxed with stirring for 15 h. The residue was extracted with chloroform, washed with water, dried over MgSO₄ and the solvent was removed in vacuum to give 59. ¹H NMR (CD₃OD): δ 1.21 (m, 12H), 2.01 (m, 15H), 4.20 (m, 8H). ³¹P NMR (D₂O): δ 59.0.

5-[((3-Amino-3-carboxy)propyl)(hydroxy)phosphinyl]pentanoic Acid (60). 190 mg of 59 was treated with 2 ml of 8N HCl and refluxed for 15 h. The reaction mixture was concentrated under vacuum and the residue was purified using Dowex AG50×4 cation exchange resin column (H⁺, 20-50 mesh, 24×1.7 cm, water elution). The fractions which gave positive color reaction with ninhydrine were combined and evaporated under vacuum to give 60. ¹H NMR (D₂O): δ 1.66 (m, 8H), 2.09 (m, 2H), 2.06 (m, 2H), 2.38 (t, J=7.2 Hz, 2H), 3.94 (t, J=5.93 Hz, 1H). ³¹P NMR (D₂O): δ 60.58.

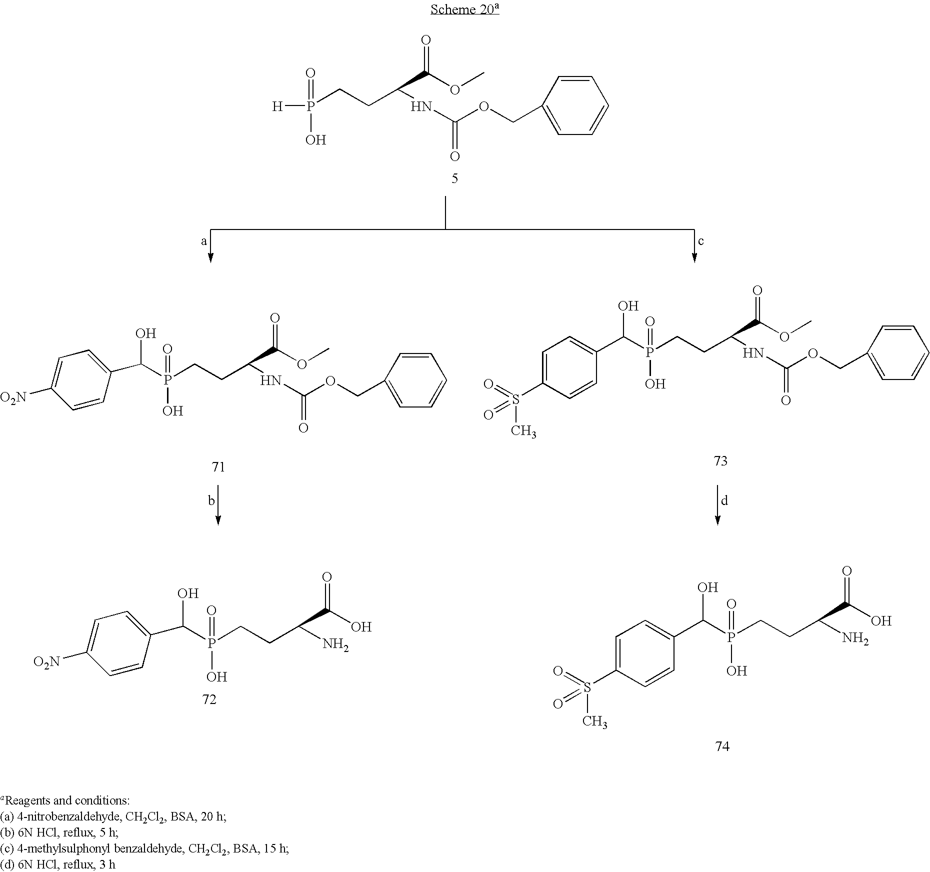

(3S)-4-[(((3-(N-Benzyloxycarbonyl)amino-3-methoxycarbonyl)propyl) (hydroxy) phosphinyl)-hydroxymethyl] nitrobenzene (71). The compound was prepared from 5 (0.8 mmol) and 4-nitrobenzaldehyde (302 mg, 2 mmol) by using the procedure described for preparation of compound 15. ¹H NMR (CD₃OD): δ 1.98 (m, 4H), 3.73 (s, 3H), 4.32 (m, 1H), 5.12 (s, 2H), 5.19 (d, J=12.56 Hz, 1H), 7.33 (m, 5H), 7.71 (m, 2H), 8.19 (d, J=8.32 Hz, 2H). ³¹P NMR (CD₃OD): δ 48.26.

(3S)-4-[(((3-amino-3-carboxy)propyl)(hydroxy)phosphinyl)-hydroxymethyl] nitrobenzene (72). The removal of the protecting groups in compound 71 was accomplished following the same procedure as that followed for compound 16 and purified by Dowex AG50×4 column to afford 72 (quantitative yield). ¹H NMR (D₂O): δ 1.72 (m, 2H), 2.11 (m, 2H), 4.02 (m, 1H), 4.97 (d, J=10.8 Hz, 1H), 7.58 (d, J=7.43 Hz, 2H), 8.18 (d, J=8.64 Hz, 2H). ³¹P NMR (D₂O): δ 49.43. ¹³C NMR (D₂O): δ 22.79 (d, J=98.03 Hz), 23.57, 54.02, 73.05 (d, J=104.90 Hz), 123.89, 127.98, 146.45, 147.40, 172.27. Mass (ESI): 319.1 (M+1).

(3S)-4-[(((3-(N-Benzyloxycarbonyl)amino-3-methoxycarbonyl)propyl) (hydroxy) phosphinyl)-hydroxymethyl]methylsulphonylbenzene (73). The compound was prepared from 5 (0.8 mmol) and 4-methylsulphonylbenzaldehyde (276 mg, 1.5 mmol) by following the procedure described for preparation of compound 15. ¹H NMR (CD₃OD): δ 2.03 (m, 4H), 3.11 (s, 3H), 3.72 (s, 3H), 4.32 (m, 1H), 5.12 (s, 2H), 5.14 (d, J=7.12 Hz, 1H), 7.35 (m, 5H), 7.76 (d, J=7.31 Hz, 2H), 7.96 (d, J=8.17 Hz, 2H). ³¹P NMR (CD₃OD): δ 48.37. ¹³C NMR (CD₃OD): δ 22.10 (d, J=91.02 Hz), 24.19, 43.65, 52.20, 55.01, 66.95, 71.87 (d, J=108.88 Hz), 127.28, 128.01, 128.28, 128.72, 137.10, 140.17, 144.50, 157.61, 172.86.

(3S)-4-[(((3-amino-3-carboxy)propyl)(hydroxy)phosphinyl)-hydroxymethyl]methylsulphonylbenzene (74). The removal of the protecting groups in compound 73 was accomplished following the same procedure as that followed for compound 16 and purified by Dowex AG50×4 column to afford 74 (quantitative yield). ¹H NMR (D₂O): δ 1.69 (m, 2H), 2.07 (m, 2H), 3.16 (s, 3H), 3.99 (m, 1H), 4.93 (d, J=10.63 Hz, 1H), 7.59 (d, J=8.31 Hz, 2H), 7.84 (d, J=8.33 Hz, 2H). ³¹P NMR (D₂O): δ 49.72. ¹³C NMR (D₂O): δ 21.69 (d, J=88.19 Hz), 22.87, 43.66, 53.21, 71.92 (d, J=107.75 Hz), 127.54, 128.26, 138.65, 143.91, 171.42.

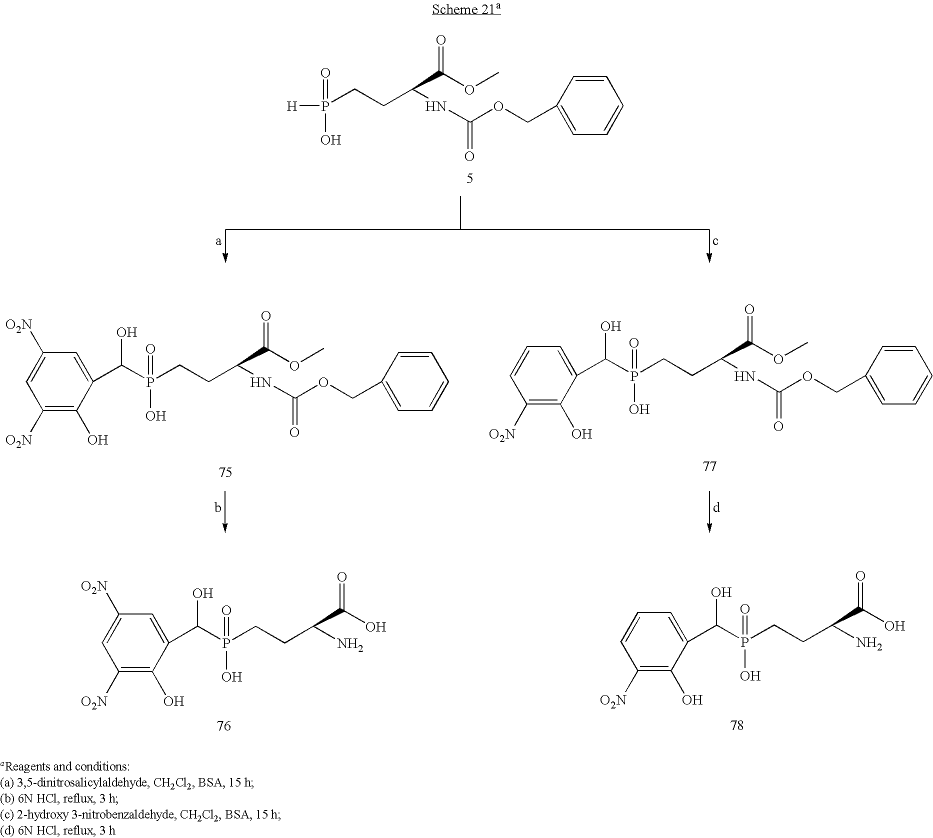

(3S)-3-[(((3-(N-Benzyloxycarbonyl)amino-3-methoxycarbonyl)propyl) (hydroxy) phosphinyl)-hydroxymethyl] 2-hydroxy-1,5-dinitrobenzene (75). The compound was prepared from 5 (0.8 mmol) and 3,5-dinitrosalicylaldehyde (424 mg, 2 mmol) by using the procedure described for preparation of compound 15. ¹H NMR (CD₃OD): δ 2.04 (m, 4H), 3.75 (s, 3H), 4.29 (m, 1H), 5.07 (s, 2H), 5.55 (d, J=10.45 Hz, 1H), 7.31 (m, 5H), 8.64 (m, 1H), 8.88 (m, 1H). ³¹P NMR (CD₃OD): δ 48.38.

(3S)-3-[(((3-amino-3-carboxy)propyl)(hydroxy)phosphinyl)-hydroxymethyl] 2-hydroxy-1,5-dinitrobenzene (76). The removal of the protecting groups in compound 75 was accomplished following the same procedure as that followed for compound 16 and purified by Dowex AG50×4 column to afford 76 (quantitative yield). ¹H NMR (D₂O): δ 1.84 (m, 2H), 2.18 (m, 2H), 4.04 (m, 1H), 5.37 (d, J=8.28 Hz, 1H), 8.57 (s, 1H), 8.93 (s, 1H). ³¹P NMR (D₂O): 6:49.52. ¹³C NMR (D₂O): δ 23.39 (d, J=98.75 Hz), 23.65, 54.3, 67.2 (d, J=106.3 Hz), 121.67, 129.67, 132.29, 134.48, 139.74, 156.2, 172.39. Mass (ESI): 381.1 (M+1).

(3S)-3-[(((3-(N-Benzyloxycarbonyl)amino-3-methoxycarbonyl)propyl) (hydroxy) phosphinyl)-hydroxymethyl] 2-hydroxy-nitrobenzene (77). The compound was prepared from 5 (0.8 mmol) and 2-hydroxy-3-nitrobenzaldehyde (334 mg, 2 mmol) by following the procedure described for preparation of compound 15. ¹H NMR (CD₃OD): δ 1.96 (m, 4H), 3.73 (s, 3H), 4.29 (m, 1H), 5.12 (s, 2H), 5.55 (d, J=7.89 Hz, 1H), 7.08 (t, J=8.05 Hz, 1H), 7.33 (m, 5H), 7.95 (d, J=7.45 Hz, 1H), 8.06 (d, J=8.36 Hz, 1H). ³¹P NMR (CD₃OD): δ 48.74. ¹³C NMR (CD₃OD): δ 21.76 (d, J=88.40 Hz), 24.20, 52.09, 55.04, 65.25 (d, J=111.58 Hz), 66.87, 119.89, 124.68, 127.88, 128.13, 128.58, 129.45, 134.32, 136.57, 137.07, 151.83, 157.62, 172.82.

(3S)-3-[(((3-amino-3-carboxy)propyl)(hydroxy)phosphinyl)-hydroxymethyl] 2-hydroxy-nitrobenzene (78). The removal of the protecting groups in compound 77 was accomplished following the same procedure as that followed for compound 16 and purified by Dowex AG50×4 column to afford 78 (quantitative yield). ¹H NMR (D₂O): δ 1.83 (m, 2H), 2.12 (m, 2H), 4.08 (m, 1H), 5.34 (d, J=7.74 Hz, 1H), 7.06 (t, J=8.14 Hz, 1H), 7.78 (d, J=7.59 Hz, 1H), 8.03 (d, J=8.50 Hz, 1H). ³¹P NMR (D₂O): δ 50.72. ¹³C NMR (20): δ 23.19 (d, J=89.51 Hz), 23.58, 53.91, 66.31 (d, J=108.25 Hz), 120.45, 125.17, 129.22, 134.59, 136.51, 151.51, 172.41.

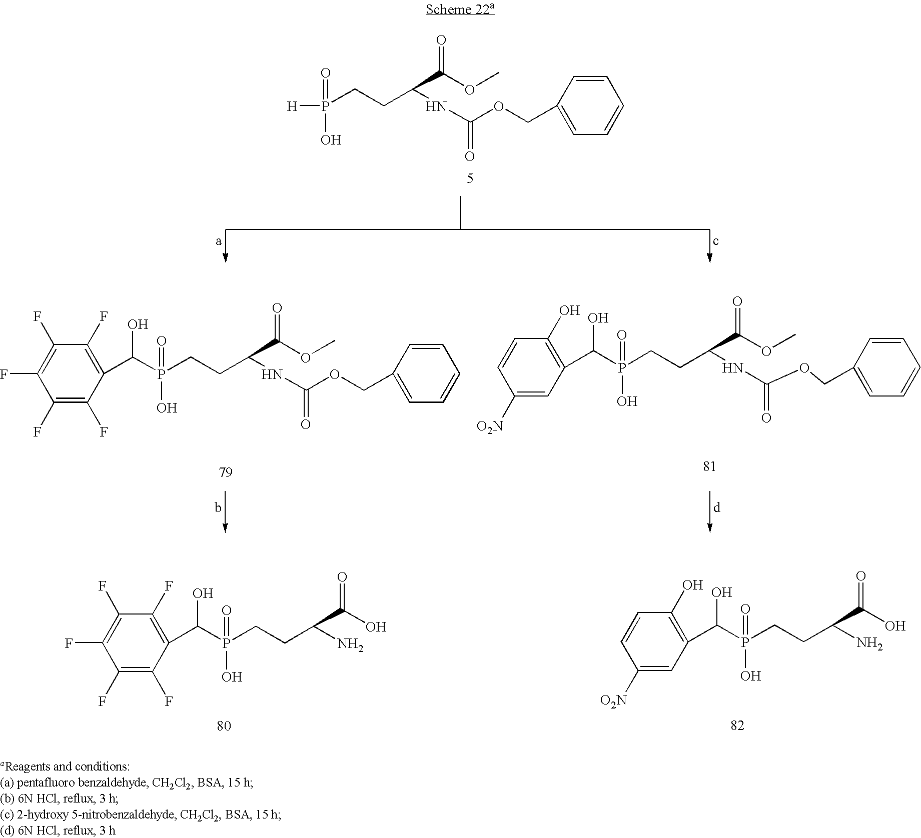

(3S)-1-[(((3-(N-Benzyloxycarbonyl)amino-3-methoxycarbonyl)propyl) (hydroxy) phosphinyl)-4-hydroxymethyl] 2,3,4,5,6-pentafluorobenzene (79). The compound was prepared from 5 (0.8 mmol) and pentafluoro benzaldehyde (392 mg, 2 mmol) by using the procedure described for preparation of compound 15. ¹H NMR (CD₃OD): δ 2.16 (m, 4H), 3.76 (s, 3H), 4.32 (m, 1H), 5.12 (s, 2H), 5.34 (d, J=11.02 Hz, 1H), 7.33 (m, 5H). ³¹P NMR (CD₃OD): δ 47.51.

(3S)-1-[(((3-amino-3-carboxy)propyl)(hydroxy)phosphinyl)-hydroxymethyl] 2,3,4,5,6-pentafluorobenzene (80). The removal of the protecting groups in compound 79 was accomplished following the same procedure as that followed for compound 16 and purified by Dowex AG50×4 column to afford 80 (quantitative yield). ¹H NMR (D₂O): δ 1.86 (m, 2H), 2.13 (m, 2H), 4.07 (m, 1H), 5.18 (d, J=10.68 Hz, 1H). ³¹P NMR (D₂O): δ 47.78. Mass (ESI): 364.1 (M+1).

(3S)-3-[(((3-(N-Benzyloxycarbonyl)amino-3-methoxycarbonyl)propyl) (hydroxy) phosphinyl)-hydroxymethyl] 4-hydroxy-nitrobenzene (81). The compound was prepared from 5 (0.8 mmol) and 2-hydroxy-5-nitrobenzaldehyde (334 mg, 2 mmol) by following the procedure described for preparation of compound 15. ¹H NMR (CD₃OD): δ 2.05 (m, 4H), 3.72 (s, 3H), 4.30 (m, 1H), 5.12 (s, 2H), 5.45 (d, J=7.41 Hz, 1H), 6.96 (d, J=8.95 Hz, 1H), 7.30 (m, 5H), 8.07 (m, 1H), 8.47 (s, 1H). ³¹P NMR (CD₃OD): δ 49.56.

(3S)-3-[(((3-amino-3-carboxy)propyl)(hydroxy)phosphinyl)-hydroxymethyl] 4-hydroxy-nitrobenzene (82). The removal of the protecting groups in compound 81 was accomplished following the same procedure as that followed for compound 16 and purified by Dowex AG50×4 column to afford 82 (quantitative yield). ¹H NMR (D₂O): δ 1.83 (m, 2H), 2.09 (m, 2H), 3.98 (m, 1H), 5.10 (d, J=8.22 Hz, 1H), 6.83 (d, J=9.01 Hz, 1H), 7.91 (d, J=8.93 Hz, 1H), 8.16 (s, 1H). ³¹P NMR (D₂O): δ 51.73. ¹³C NMR (D₂O): δ 20.90 (d, J=76.74 Hz), 21.51, 51.95 (d, J=13.02 Hz), 65.72 (d, J=108.25 Hz), 114.91, 122.96, 123.66, 124.20, 138.82, 158.63, 170.29. Mass (ESI): 335.1 (M+1).

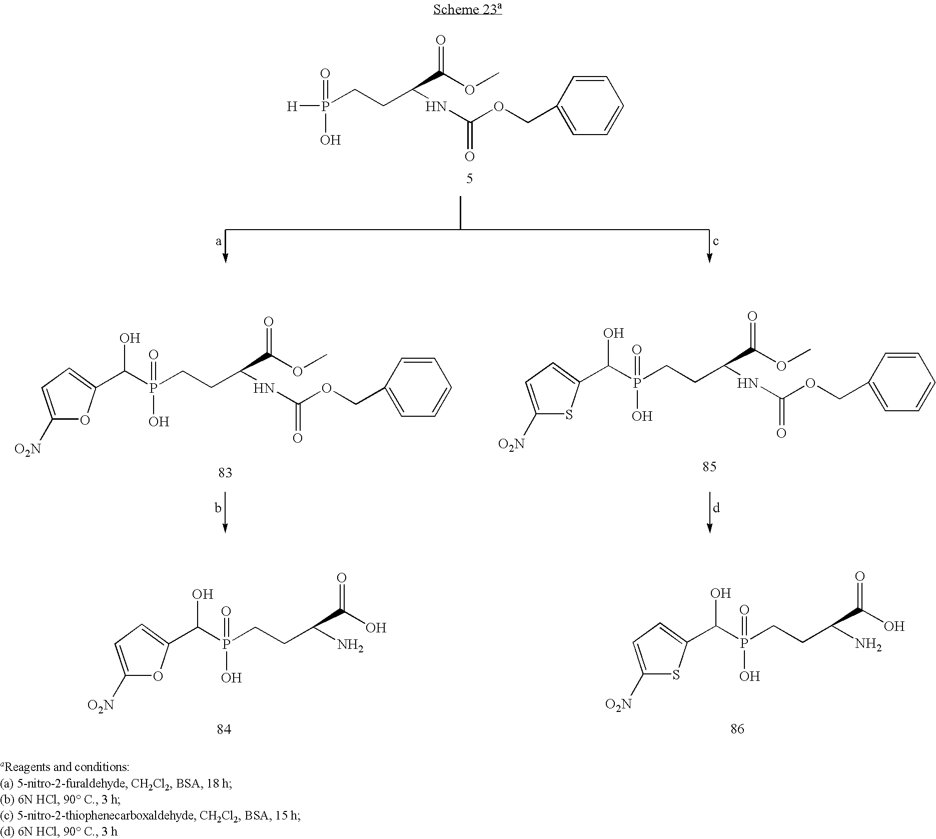

(3S)-2-[(((3-(N-Benzyloxycarbonyl)amino-3-methoxycarbonyl)propyl) (hydroxy) phosphinyl)-hydroxymethyl] 5-nitrofuran (83). The compound was prepared from 5 (0.8 mmol) and 5-nitro-2-furaldehyde (282 mg, 2 mmol) by using the procedure described for preparation of compound 15. ¹H NMR (CD₃OD): δ 2.07 (m, 4H), 3.73 (s, 3H), 4.32 (m, 1H), 5.08 (d, J=15.98 Hz, 1H), 5.11 (s, 2H), 6.80 (m, 1H), 7.38 (m, 6H). ³¹P NMR (CD₃OD): δ 46.14.

(3S)-2-[(((3-amino-3-carboxy)propyl)(hydroxy)phosphinyl)-hydroxymethyl] 5-nitrofuran (84). The removal of the protecting groups in compound 83 was accomplished following the same procedure as that followed for compound 16 and purified by Dowex AG50×4 column to afford 84 (quantitative yield). ¹H NMR (D₂O): δ 1.86 (m, 2H), 2.19 (m, 2H), 4.12 (m, 1H), 4.95 (d, J=11.96 Hz, 1H), 6.73 (m, 1H), 7.50 (d, J=3.69 Hz, 1H). ³¹P NMR (D₂O): δ 48.16. Mass (ESI): 307.1 (M+1).

(3S-2-[(((3-(N-Benzyloxycarbonyl)amino-3-methoxycarbonyl)propyl) (hydroxy) phosphinyl)-hydroxymethyl] 5-nitrothiophene (85). The compound was prepared from 5 (0.8 mmol) and 5-nitro-2-thiophenecarboxaldehyde (314 mg, 2 mmol) by following the procedure described for preparation of compound 15. ¹H NMR (CD₃OD): δ 2.08 (m, 4H), 3.72 (s, 3H), 4.30 (m, 1H), 5.10 (m, 3H), 7.12 (m, 1H), 7.34 (m, 5H), 7.89 (m, 1H). ³¹P NMR (CD₃OD): δ 46.65.

(3S)-2-[(((3-amino-3-carboxy)propyl)(hydroxy)phosphinyl)-hydroxymethyl] 5-nitrothiophene (86). The removal of the protecting groups in compound 85 was accomplished following the same procedure as that followed for compound 16 and purified by Dowex AG50×4 column to afford 86 (quantitative yield). ¹H NMR (D₂O): δ 1.78 (m, 2H), 2.14 (m, 2H), 4.02 (m, 1H), 5.08 (d, J=11.11 Hz, 1H), 7.06 (m, 1H), 7.94 (d, J=4.30 Hz, 1H). ³¹P NMR (D₂O): δ 46.82. ¹³C NMR (D₂O): δ 22.83 (d, J=93.34 Hz), 23.70, 53.89, 70.19 (d, J=106.74 Hz), 124.93, 130.71, 150.24, 153.80, 172.35. Mass (ESI): 323.1 (M−1).

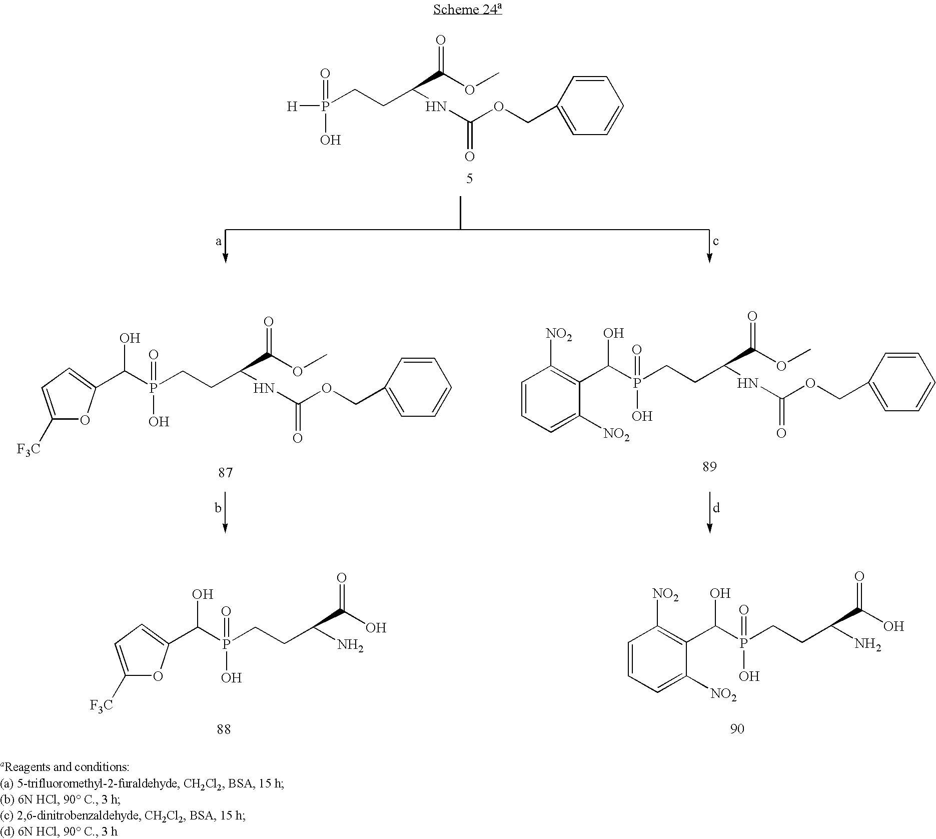

(3S)-2-[(((3-(N-Benzyloxycarbonyl)amino-3-methoxycarbonyl)propyl) (hydroxy) phosphinyl)-hydroxymethyl] 5-trifluoromethylfuran (87). The compound was prepared from 5 (0.8 mmol) and 5-trifluoromethyl-2-furaldehyde (328 mg, 2 mmol) by using the procedure described for preparation of compound 15. ¹H NMR (CD₃OD): δ 2.01 (m, 4H), 3.72 (s, 3H), 4.32 (m, 1H), 5.03 (d, J=11.24 Hz, 1H), 5.12 (s, 2H), 6.70 (m, 1H), 6.94 (m, 1H), 7.32 (m, 5H). ³¹P NMR (CD₃OD): δ 46.79.

(3S)-2-[(((3-amino-3-carboxy)propyl)(hydroxy)phosphinyl)-hydroxymethyl] 5-trifluoromethylfuran (88). The removal of the protecting groups in compound 87 was accomplished following the same procedure as that followed for compound 16 and purified by Dowex AG50×4 column to afford 88 (quantitative yield). ¹H NMR (D₂O): δ 1.82 (m, 2H), 2.18 (m, 2H), 4.07 (m, 1H), 4.85 (d, J=11.59 Hz, 1H), 6.53 (m, 1H), 6.59 (m, 1H). ³¹P NMR (D₂O): δ 48.29. ¹³C NMR (D₂O): δ 23.11 (d, J=91.21 Hz), 23.41, 53.82 (d, J=13.90 Hz), 67.16 (d, J=109.70 Hz), 110.80, 113.74, 115.53 (q, J=266.26 Hz), 141.40 (q, J=43.59 Hz), 154.59, 172.09.

(3S)-2-[(((3-(N-Benzyloxycarbonyl)amino-3-methoxycarbonyl)propyl) (hydroxy) phosphinyl)-hydroxymethyl] 1,3-dinitrobenzene (89). The compound was prepared from 5 (0.8 mmol) and 2,6-dinitrobenzaldehyde (392 mg, 2 mmol) by following the procedure described for preparation of compound 15. ¹H NMR (CD₃OD): δ 1.96 (m, 4H), 3.72 (s, 3H), 4.32 (m, 1H), 5.11 (s, 2H), 6.27 (d, J=16.15 Hz, 1H), 7.34 (m, 5H), 7.64 (m, 1H), 7.97 (d, J=7.86 Hz, 2H). ³¹P NMR (CD₃OD): δ 48.69.

(3S)-2-[(((3-amino-3-carboxy)propyl)(hydroxy)phosphinyl)-hydroxymethyl] 1,3-dinitro benzene (90). The removal of the protecting groups in compound 89 was accomplished following the same procedure as that followed for compound 16 and purified by Dowex AG50×4 column to afford 90 (quantitative yield). ¹H NMR (D₂O): δ 1.87 (m, 2H), 2.12 (m, 2H), 4.03 (m, 1H), 5.96 (d, J=11.57 Hz, 1H), 7.64 (t, J=7.97, 1H), 8.01 (d, J=6.70 Hz, 2H). ³¹P NMR (D₂O): δ 48.28. ¹³C NMR (D₂O): δ 23.80, 24.55 (d, J=93.91 Hz), 54.29, 69.17 (d, J=100.70 Hz), 128.81, 129.65, 149.49, 172.54. Mass (ESI): 364.1 (M+1).

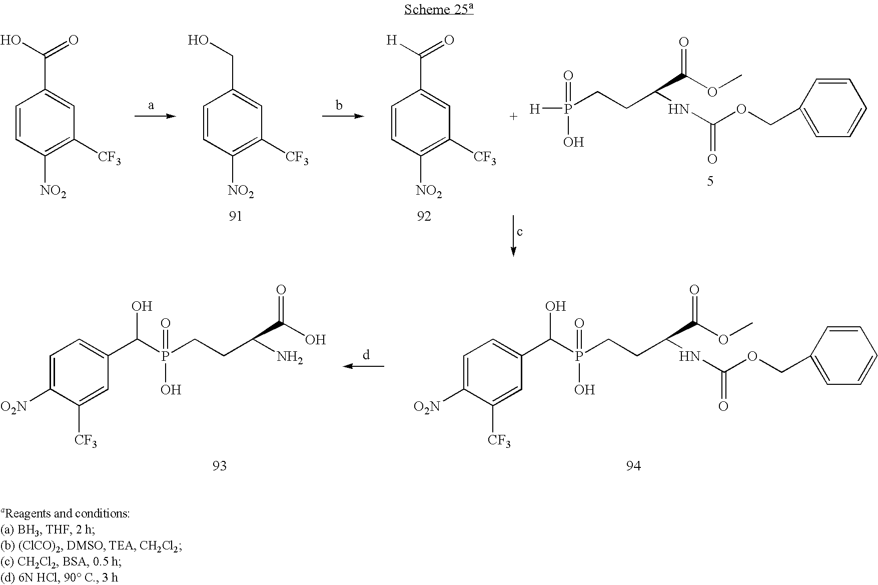

3-trifluoromethyl-4-nitrobenzyl alcohol (91). To a stirred solution of 3-trifluoromethyl-4-nitrobenzoic acid (3 g) in 15 ml of tetrahydrofuran at 0° C. was added 1 M BH₃/THF (64 ml) dropwise under argon. This reaction mixture was allowed to stir at room temperature for 2 h and quenched by saturated NaHCO₃. The solution was extracted with dichloromethane and then evaporated the organic layer to dryness in vacuo. The crude residue was purified on silica gel using cyclohexane:ethylacetate (90:10 to 60:40, gradient) as the eluent to afford 1.76 g of 91. ¹H NMR (CD₃OD): δ 4.80 (s, 2H), 7.78 (d, J=8.33 Hz, 1H), 4.91 (m, 2H).

3-trifluoromethyl-4-nitrobenzaldehyde (92). Dichloromethane (7 ml) was cooled to −78° C. in round bottom flak with septum under argon. Oxalyl chloride (0.49 ml) was added in one portion. Dimethyl sulfoxide (0.67 ml) in dichloromethane (3.5 ml) was added dropwise over 1 h. 3-trifluoromethyl-4-nitrobenzyl alcohol 91 (1.04 g) in dichloromethane (7 ml) was added dropwise over 1 h. The reaction mixture was stirred at −78° C. for 45 min. Triethylamine (2.6 ml) was added over 45 min. TLC analysis indicated the reaction was complete. The reaction was quenched with 1 M aqueous potassium hydrogensulfate (50 ml) the organic layer was washed with saturated NaHCO₃ (50 ml), water (50 ml), and brine (50 ml). The organic layer was dried over magnesium sulphate, and concentrated in vacuo to afford the desired aldehyde 92. ¹H NMR (CDCl₃): δ 8.05 (d, J=8.2 Hz, 1H), 8.28 (d, J=8.4 Hz, 1H), 8.36 (s, 1H), 10.18 (s, 1H).

(3S)-4-[(((3-(N-Benzyloxycarbonyl)amino-3-methoxycarbonyl)propyl) (hydroxy) phosphinyl)-hydroxymethyl] 2-trifluoromethyl-1-nitrobenzene (93). The compound was prepared from 5 (0.8 mmol) and 3-trifluoromethyl-4-nitrobenzaldehyde (394 mg, 1.8 mmol) by following the procedure described for preparation of compound 15. ¹H NMR (CD₃OD): δ 2.05 (m, 4H), 3.74 (s, 3H), 4.32 (m, 1H), 5.11 (s, 2H), 5.18 (d, J=10.39 Hz, 1H), 7.34 (m, 5H), 8.01 (m, 3H). ³¹P NMR (CD₃OD): δ 47.70.

(3S)-4-[(((3-amino-3-carboxy)propyl)(hydroxy)phosphinyl)-hydroxymethyl] 2-trifluoro methyl-1-nitrobenzene (94). The removal of the protecting groups in compound 93 was accomplished following the same procedure as that followed for compound 16 and purified by Dowex AG50×4 column to afford 94 (quantitative yield). ¹H NMR (D₂O): δ 1.79 (m, 2H), 2.14 (m, 2H), 4.03 (m, 1H), 5.02 (d, J=10.85 Hz, 1H), 7.82 (d, J=8.42 Hz, 1H), 7.94 (s, 1H), 8.02 (d, J=8.43 Hz, 1H). ³¹P NMR (D₂O): δ 48.33. Mass (ESI): 387.1 (M+1).

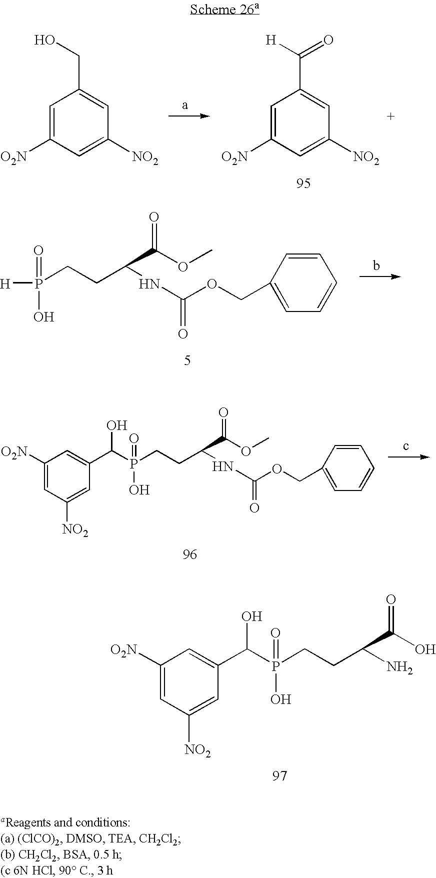

3,5-dinitrobenzaldehyde (95). The title compound was obtained from 3,5-dinitrobenzyl alcohol as yellow solid in a similar manner for the preparation of 92. ¹H NMR (CDCl₃): δ 9.04 (s, 2H), 9.22 (s, 1H), 10.25 (s, 1H).

(3S)-3-[(((3-(N-Benzyloxycarbonyl)amino-3-methoxycarbonyl)propyl) (hydroxy) phosphinyl)-hydroxymethyl] 1,5-dinitrobenzene (96). The compound was prepared from 5 (0.8 mmol) and 3,5-dinitrobenzaldehyde (392 mg, 2 mmol) by using the procedure described for preparation of compound 15. ¹H NMR (CD₃OD): δ 1.99 (m, 4H), 3.75 (s, 3H), 4.32 (m, 1H), 5.10 (s, 2H), 5.29 (d, J=9.43 Hz, 1H), 7.32 (m, 5H), 8.72 (s, 2H), 8.88 (s, 1H). ³¹P NMR (CD₃OD): δ 47.71. ¹³C NMR (CD₃OD): δ 22.28 (d, J=92.43 Hz), 24.10, 52.19, 54.96, 66.92, 70.65 (d, J=108.91 Hz), 117.79, 127.30, 127.86, 128.15, 128.59, 136.98, 143.01, 148.60, 157.60, 172.83.

(3S)-3-[(((3-amino-3-carboxy)propyl)(hydroxy)phosphinyl)-hydroxymethyl] 1,5-dinitrobenzene (97). The removal of the protecting groups in compound 96 was accomplished following the same procedure as that followed for compound 16 and purified by Dowex AG50×4 column to afford 97 (quantitative yield). ¹H NMR (D₂O): δ 1.78 (m, 2H), 2.14 (m, 2H), 4.05 (m, 1H), 5.08 (d, J=10.04 Hz, 1H), 8.58 (s, 2H), 8.91 (s, 1H). ³¹P NMR (D₂O): δ 48.29. ¹³C NMR (D₂O): δ 22.81 (d, J=91.46 Hz), 23.54, 53.84, 71.99 (d, J=104.79 Hz), 118.30, 127.49, 143.41, 148.37, 172.17. Mass (ESI): 364.0 (M+1).

### Example 2

### Synthesis of Substituted Benzaldehydes

A) Preparation of Nitro-Benzaldehydes from Nitro-Benzoic Acids or Nitro-Benzyl Alcohols

1) Reduction (Step 1)—Oxidation (Step 2)

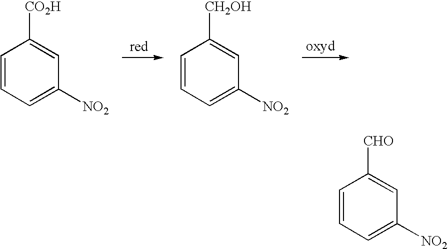

### Reduction Step:

### a) BH3-SMe2 (Aulenta JOC 05; Campbell TL03)

### b) BH3, THF (Campbell TL03; Liou JMC04; Parlow JMC03)

### Oxidation Step:

### a) PDC (Liou JMC04)

### b) PCC (Aulenta JOC05; Campbell TL03)

c) oxidizing polymer (Sorg Angew 01)

### d) Swern (Campbell 03; Parlow JMC03)

### 2) One Step Reduction

### i) TMSCl ii) DiBAL-H (Chandrasekhar TL98)

This procedure was applied to the following alcohols or acids:

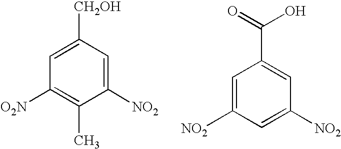

### B) Substitutions of Nitro-Benzaldehydes

1) Substitution with Benzofurazans

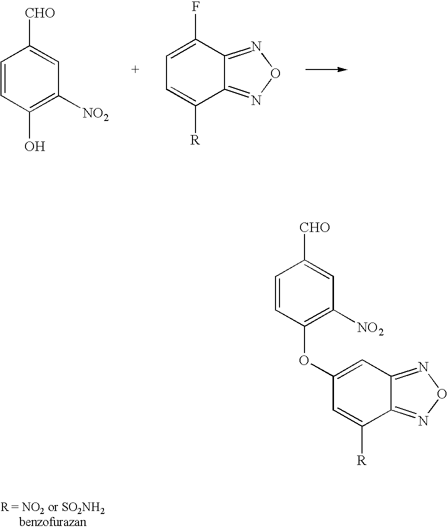

2) Substitution with Sulfonyl Chlorides

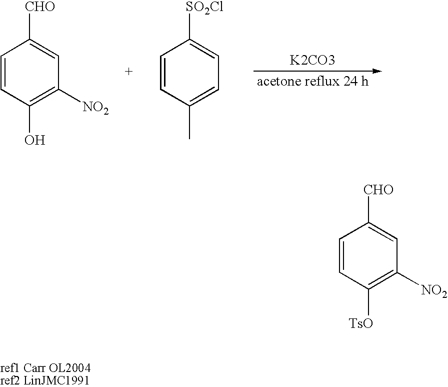

other sulfonylchlorides can be used, for example

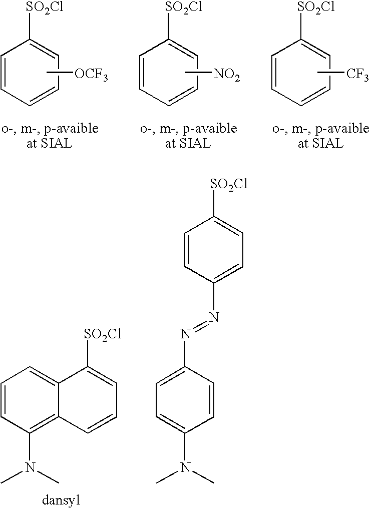

The following phosphinates can be synthesized using the aldehydes described above

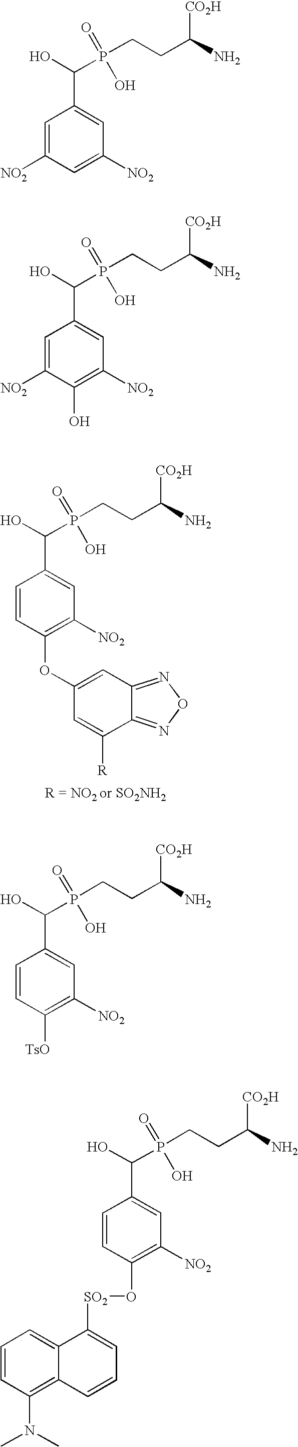

### Example 3

### Experimental of Hypophosphorous Acid Derivatives According to Method B

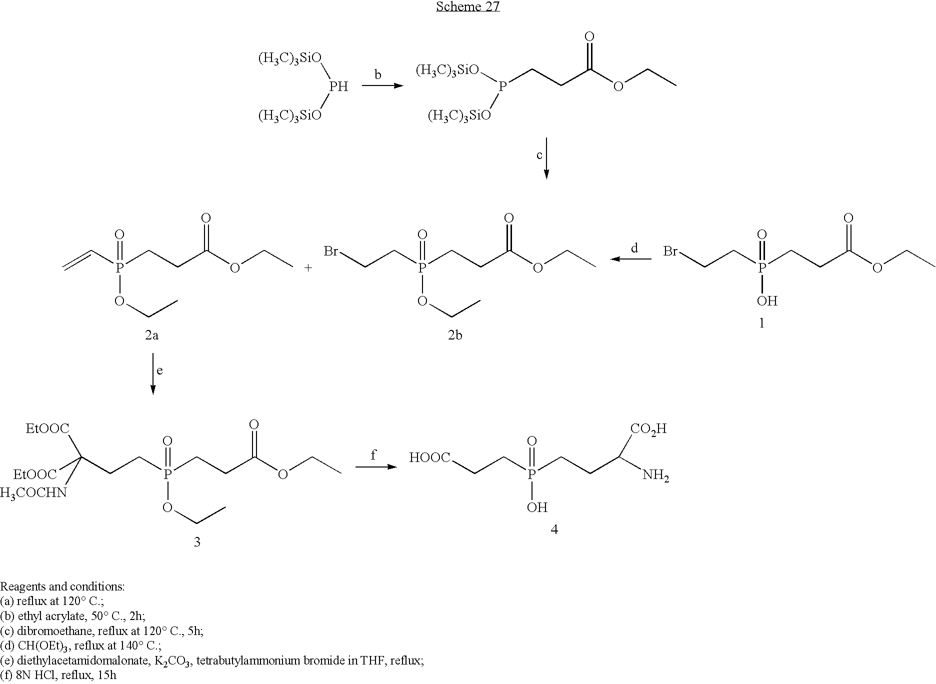

3-[(2-Bromoethyl)(ethoxy)phosphinyl)]propanoic Acid Ethyl Ester (1). A mixture of ammonium hypophosphite (4 g, 48 mmol) and hexamethydisilazane (7.73 g, 48 mmol) was heated at 120° C. for one hour under argon. After the mixture was cooled to 0° C., ethyl acrylate (4.8 g, 48 mmol) was carefully added dropwise and the resulting mixture was stirred at 50° C. for 2 h. Then the mixture was cooled to room temperature, dibromoethane (20 ml) was added and stirred for 5 h at 120° C. The formed trimethylbromosilane and excess dibromoethane were removed under vacuum. Then 50 ml of aqueous ethanol (1:1) were added dropwise to the residue and refluxed for 0.5 h. Then the solvent was removed under vacuum and extracted with ethyl acetate. The organic layer was dried over MgSO₄ and the solvent was removed in vacuum to give 1 (5.42 g, 41.4%). ¹H NMR (CD₃OD): δ 1.25 (t, J=7.1 Hz, 3H), 2.06 (m, 2H), 2.42 (m, 2H), 2.61 (m, 2H), 2.61 (m, 2H), 4.14 (q, J=7.1 Hz, 2H). ³¹P NMR (CD₃OD): δ 49.5.

3-[Ethoxy(vinyl)phosphinyl]propanoic Acid Ethyl Ester (2). 5.42 g of 1 (19.9 mmol) were treated with 40 ml of triethyl orthoformate, and the mixture was refluxed with a Dean-Stark trap to remove ethanol and ethyl formate. Excess of triethylorthoformate was removed in vacuo to give 2a+2b ([39.5:60.5], 5.91 g). 2b: ¹H NMR (CD₃OD): δ 1.27 (m, 6H), 2.18 (m, 2H), 2.57 (m, 2H), 4.10 (m, 4H), 6.36 (m, 3H). ³¹P NMR (CD₃OD): δ 44.9.

3-[((3-(N-Acetyl)amino)-3-(bisethoxycarbonyl)propyl)(ethoxy)phosphinyl]propanoic Acid Ethyl Ester (3). Compound 2 (500 mg, 0.9:1 mmol[a:b]) was mixed with diethylacetamidomalonate (453 mg, 2.1 mmol), potassium carbonate (573 mg, 4.2 mmol) and tetrabutylammonium bromide (32.2 mg, 0.1 mmol) in THF (2 ml). The reaction mixture was refluxed with stirring for 15 h. The residue was extracted with chloroform, washed with water, dried over MgSO₄ and the solvent was removed in vacuum to give 3 (564 mg, 67.9%). The residue was purified by column chromatography (Silica gel 60, EtOAc/MeOH, 1:0 to 8:2) to afford 3 (507 mg). ¹H NMR (CD₃OD): δ 1.31 (m, 2H), 1.75 (m, 2H), 2.05 (s, 3H), 2.16 (m, 2H), 2.59 (m, 4H), 4.17 (m, 8H). ¹³C NMR (CD₃OD): δ 13.5, 16.1, 21.6, 22.4 (d, J=101 Hz), 22.9 (d, J=93 Hz), 25.8, 26.7, 60.5, 61.1, 62.7, 66.8 (d, J=17 Hz), 167.6, 171.4, 172.5 (d, J=14 Hz). ³¹P NMR (CD₃OD): δ 58.1.

3-[((3-Amino-3-carboxy)propyl)(hydroxy)phosphinyl]propanoic Acid (4). 210 mg of 4 (0.48 mmol) was treated with 2 ml of 8N HCl and refluxed for 15 h. The reaction mixture was concentrated under vacuum and the residue was purified using Dowex AG50×4 cation exchange resin column (H⁺, 20-50 mesh, 24×1.7 cm, water elution). The fractions which gave positive color reaction with ninhydrine were combined and evaporated under vacuum to give 5 (95 mg, 82.8%). ¹H NMR (D₂O): δ 1.66 (m, 2H), 1.85 (m, 2H), 2.06 (m, 2H), 2.51 (m, 2H), 3.96 (t, J=5.7 Hz, 1H). ¹³C NMR (D₂O): δ 23.5, 24.3 (d, J=91 Hz), 25.0 (d, J=91 Hz), 27.3, 54.1 (d, J=15 Hz), 172.6, 177.5 (d, J=15 Hz). ³¹P NMR (D₂O): δ 57.4. MS (ESI): m/z 238.1 (M−1). Anal. (C₇H₁₄NO₆P.0.25H₂O)C, H, N.

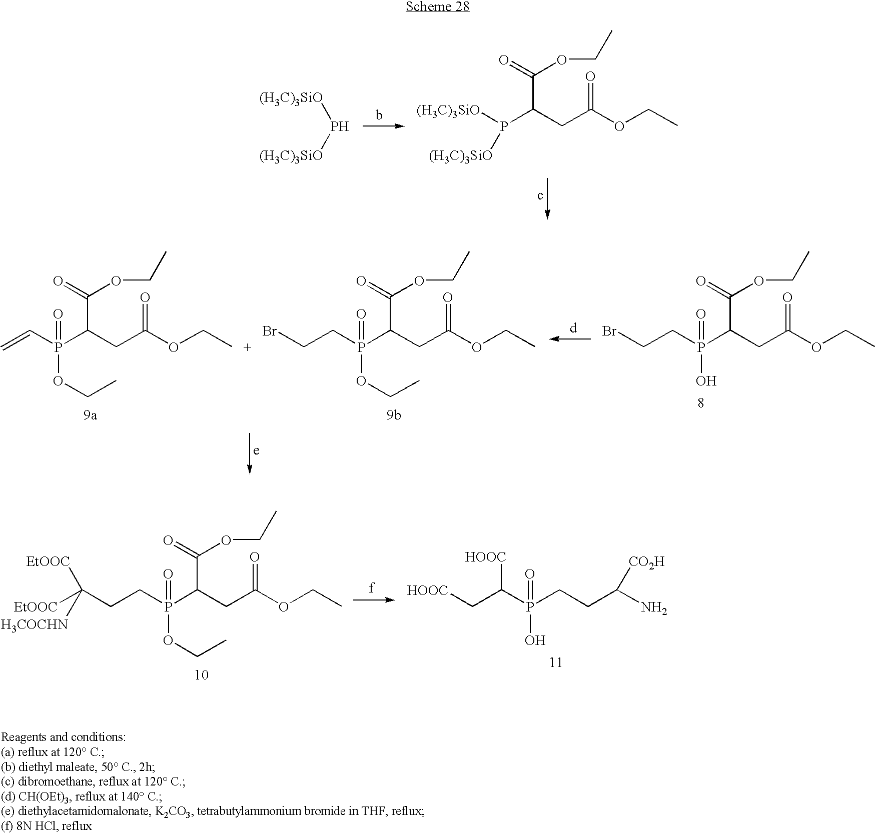

2-[((2-Bromoethyl)(hydroxy)-phosphinyl)methyl]butane-1,4-dioic Acid Ethyl Ester (8).

The compound was prepared from diethyl maleate by a procedure similar to that for the preparation of compound 1 (oily liquid, 1.21 g, 35% yield); ¹H NMR (CD₃OD): δ 1.26 (m, 6H), 2.58 (m, 2H), 2.91 (m, 2H), 3.50 (m, 1H), 3.66 (m, 2H), 4.20 (m, 4H). ³¹P NMR (CD₃OD): δ 41.9.

2-[(((3-(N-Acetyl)amino)-3-(bisethoxycarbonyl)propyl)(ethoxy)phosphinyl)methyl]butane-1,4-dioic Acid Ethyl Ester (10). Compound 8 was esterified by triethylorthoacetate by a procedure similar to that for the preparation of compound 2 (oily liquid, 1.36 g); ³¹P NMR (CD₃OD): δ 37.8, 48.1 (9a and 9b). The residue (1 g) was used for the next step procedure similar to that of compound 3 without further purification. (77.1% yield over two steps); ¹H NMR (CD₃OD): δ 1.33 (m, 15H), 1.88 (m, 2H), 2.07 (s, 3H), 2.57 (m, 2H), 2.92 (m, 2H), 3.56 (m, 1H), 4.22 (m, 10H). ³¹P NMR (CD₃OD): δ 51.7. 52.2 (1:1). MS (ESI): m/z 508.1 (M−1).

2-[(((3-amino-3-carboxy)propyl)(hydroxy)phosphinyl)-methyl]butane-1,4-dioic Acid (11). The removal of the protecting groups in compound 10 (186 mg, 0.37 mmol) was accomplished using the same procedure as that used for compound 4 to afford 11. The residue was purified by anion exchange chromatography. The residue was dissolved in freshly boiled and cooled water (0.2 L), then pH adjusted to 9-10, and the solution deposited on a AG1×4 resin (HCOO⁻, 200-400 mesh, 8.5×1 cm). The resin was washed with boiled water and the compound 10 was eluted with 0.72-0.73 M HCOOH (83 mg, 80% yield). ¹H NMR (D₂O): δ 1.78 (m, 2H), 2.14 (m, 2H), 2.81 (m, 2H), 3.18 (m, 1H), 4.05 (t, J=5.9 Hz, 1H). ¹³C NMR (D₂O): δ 23.4, 24.7 (d, J=96 Hz), 30.9, 45.0 (d, J=77 Hz), 53.7 (d, J=15 Hz), 172.0, 174.4, 176.2 (d, J=15 Hz). ³¹P NMR (D₂O): δ 46.5.

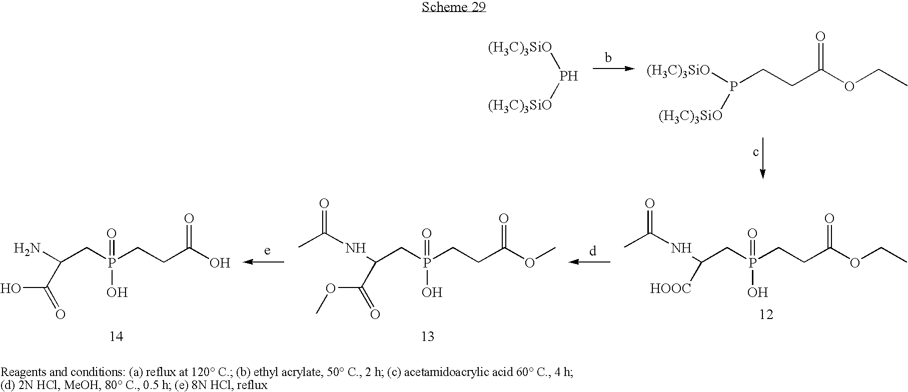

3-[(((2-(N-Acetyl)amino)-2-carboxy)propyl)(hydroxy)phosphinyl]propanoic Acid Ethyl Ester (12). A mixture of ammonium hypophosphite (498 mg, 6 mmol) and hexamethydisilazane (966 g, 6 mmol) was heated at 120° C. for one hour under argon. After the mixture was cooled to 0° C., ethyl acrylate (350 mg, 3.5 mmol) was carefully added dropwise and the resulting mixture was stirred at 50° C. for 2 h. Then the mixture was cooled to room temperature, acetamidoacrylic acid (387 mg, 3 mmol) was added and stirred for 5 h at 65° C. A sample was taken from the reaction mixture and treated with one drop of 2N HCl and CD₃OD. ¹H NMR (CD₃OD): δ 1.28 (t, J=7.1 Hz, 3H), 2.02 (s, 3H), 2.12 (m, 2H), 2.36 (m, 2H), 2.62 (m, 2H), 4.18 (q, J=7.1 Hz, 2H), 4.72 (m, 1H). ³¹P NMR (CD₃OD): δ 48.7.

3-[(((2-(N-Acetyl)amino)-2-methoxycarbonyl)propyl)(hydroxy)phosphinyl]propanoic Acid Methyl Ester (13). 10 ml of 2N HCl was added dropwise to the above residue and extracted with ethylacetate. The aqueous part was evaporated to dryness, then 50 ml of methanol were added and the solvent was removed at 50° C. under vacuum to afford 13 (645 mg, 73% yield over three steps). ¹H NMR (CD₃OD): δ 2.08 (s, 3H), 2.14 (m, 2H), 2.43 (m, 2H), 2.65 (m, 2H), 3.71 (s, 3H), 3.76 (s, 3H), 4.80 (m, 1H). ³¹P NMR (CD₃OD): δ 51.8.

3-[((2-amino-2-carboxy)propyl)(hydroxy)phosphinyl]propanoic Acid (14). The removal of the protecting groups in compound 13 (525 mg, 1.78 mmol) was accomplished following the same procedure as that followed for compound 4 to afford 14. Compound 14 was purified by a Dowex AG50×4 column as described earlier (quantitative yield). ¹H NMR (D₂O): δ 1.93 (m, 2H), 2.06 (m, 1H), 2.32 (m, 1H), 2.56 (m, 2H), 4.19 (m, 1H). ¹³C NMR (D₂O): δ 25.3 (d, J=96 Hz), 27.3, 29.4 (d, J=86 Hz), 49.3, 172.0, 177.3. ³¹P NMR (D₂O): δ 52.0. MS (ES): m/z 224.1 (M−1).

### Example 4

### Synthesis of Oxophosphonates

The α-hydroxyphosphinates described above may be oxidized to α-oxophosphinates using PDC (pyridinium dichromate) (see P. Vayron et al. Chem. Eur. J. 2000, 6, 1050)

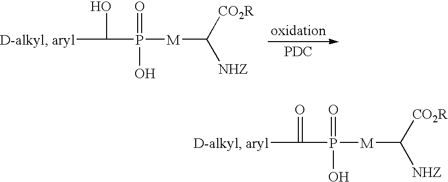

### Example 5

### Synthesis of Sulfonates

Sulfides were oxidized to sulfones using oxone. Examples are given below.

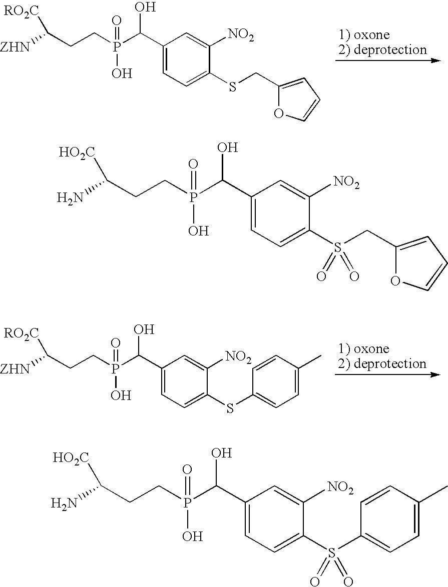

### Example 6

### Separation of α-Hydroxyphosphinate Diastereoisomers

Substituted hydroxymethyl phosphinates as 22, 24, etc. are mixtures of diastereoisomers. They were separated by HPLC using a reverse phase column (see for example Liu et al. J. Organometal. Chem. 2002, 646, 212) or a chiral anion exchange column (Chiralpack QD-AX (Daicel), see Lämmerhofer et al. Tetrahedron Asym. 2003, 14, 2557). Separation of 50 and 56 was achieved on a Crownpack column (Daicel).

### Example 7

### Cyclic Phosphinate Synthesis

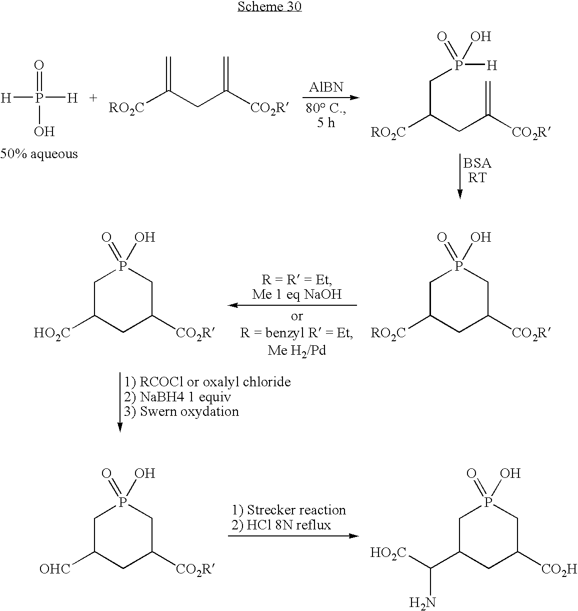

The glutaric α,γ-dimethylene diester was prepared according to Basavaiah et al. J. Org. Chem. 2002, 67, 7135

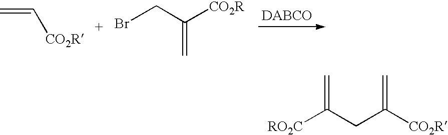

Substitutions were introduced in the starting glutaric α,γ-dimethylene diester according to Saxena et al. Synlett 2003, 10, 1439.

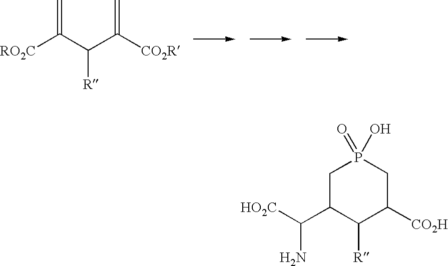

### Example 8

### Derivatives with an α, β Cyclic Aminoacid Group

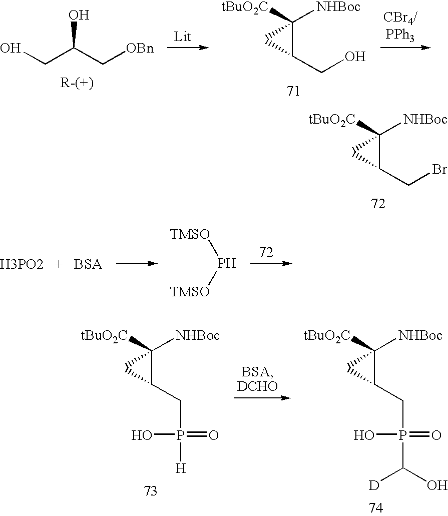

### Example 9

### Synthesis of α-Allyl Vinylglycine

Hydroxyalkylation of imidazolidinones and oxazolidinones (5, 6) derived from methionine with acetaldehyde cleanly afforded a single diastereoisomer but hydrolysis only lead to side products. However the reaction was successful with alkyl substitution: the imidazolidinone derived from methionine was converted to the vinylglycine derivative by oxidation and subsequent pyrolysis of the sulfoxide. Deprotonation of this compound followed by reaction with alkyl halides as electrophiles, cleanly afforded α-alkyl vinyl imidazolidinone which was subsequently hydrolysed (6N HCl, 100° C.) to the corresponding α-alkylated vinylglycines (Scheme 31). These compounds can be also obtained by first α-alkylation of imidazolidinones derived from methionine, and then oxidation and subsequent pyrolysis of the sulfoxide (8) (scheme 32). In this latter case diastereoisomeric excess are higher.

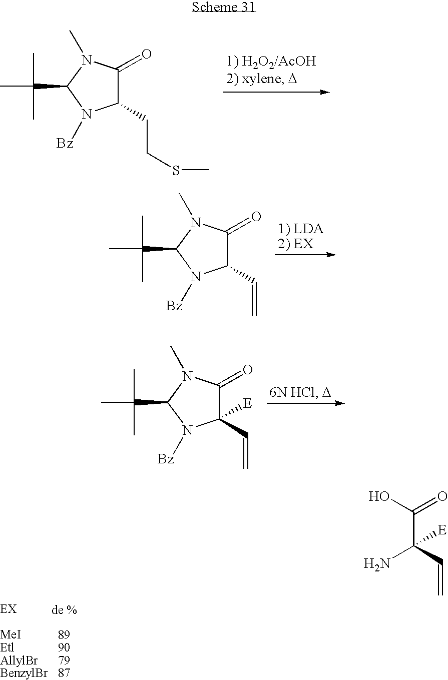

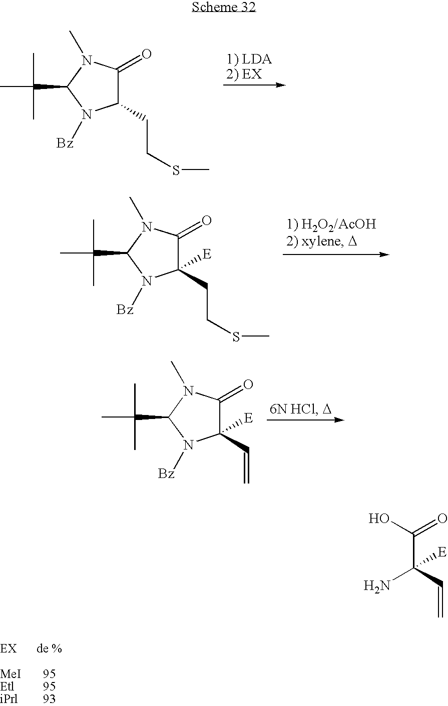

Milder hydrolysis conditions are required with oxazolidinones intermediates. This approach was used by Acton and Jones starting with D-Methionine (9). The ratio of diastereoisomeric alkyl oxazolidinone was only 88:12 and the major cis isomer could only be purified by RP HPLC. Alkylation yield was only about 50%.

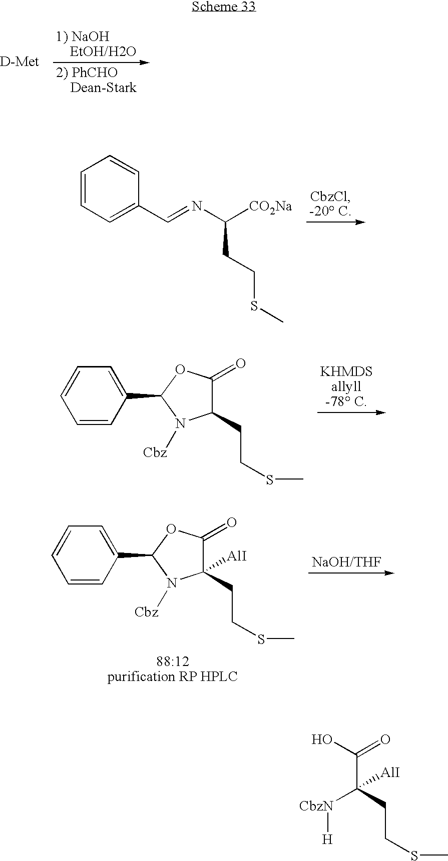

A similar methodology was recently used by Annedi et al. (10) for the synthesis of α-alkylhomoserine and could be used for α-alkylvinylglycine preparation (Scheme 34).

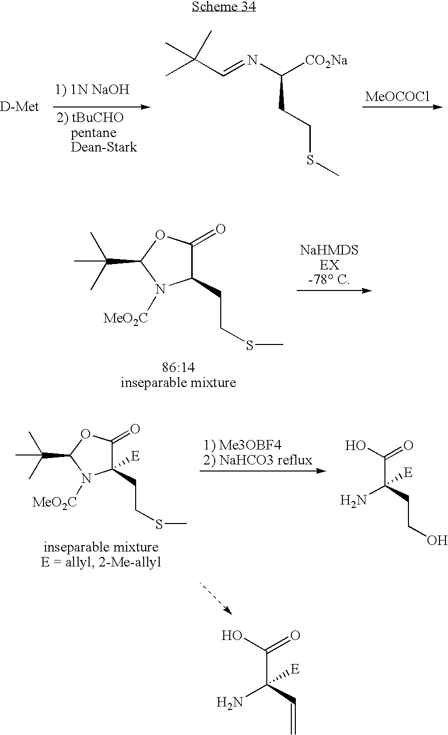

L-α-benzyl vinyl glycine may be obtained from D-Phe according to the procedure described by Cheng et al (11) (Scheme 35). A similar synthesis was carried out starting with N-protected phenylglycine (12).

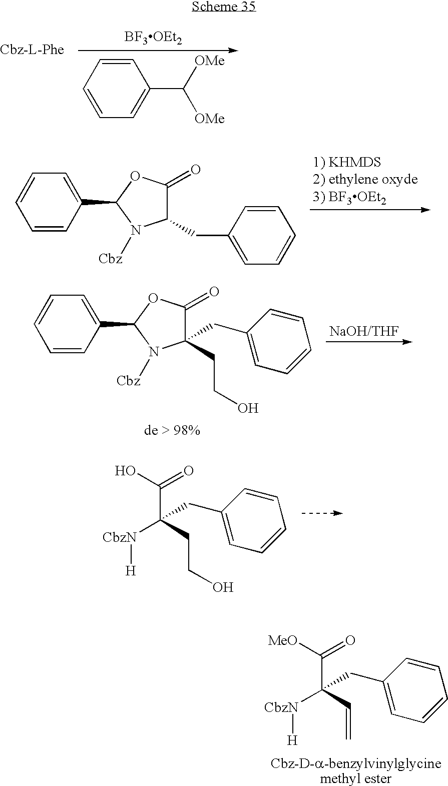

The bis-lactim methodology developed by Schöllkopf has been also used for the preparation of substituted L-vinylalanine (6, 13) (Scheme 36).

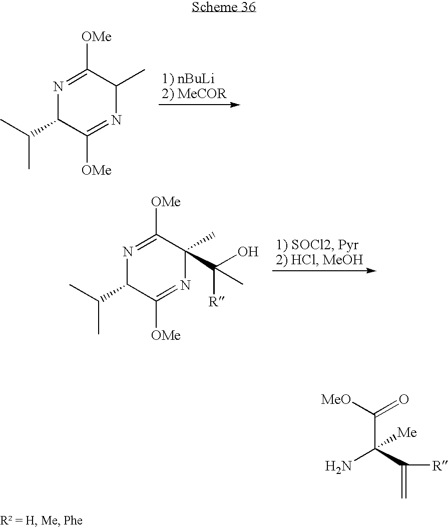

Other synthesis are reviewed and described in (6).

One possible synthesis for Cbz-L-α-alkylvinylglycine methyl ester is the following:

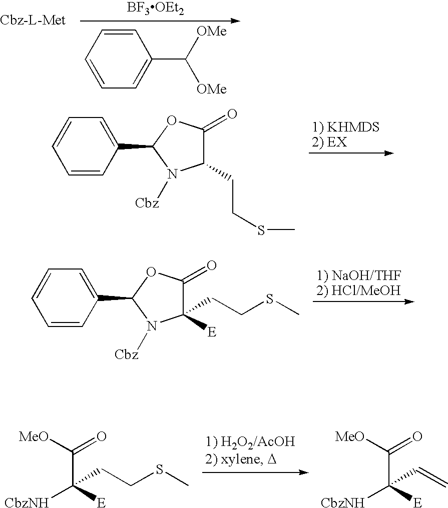

### Example 10

### Pharmacological Results

Agonist activity of the compounds was tested on HEK293 cells transiently transfected with rat mGlu4 expressing plasmid pRKG4 and chimeric G-protein Gqi9 by electroporation, as described by Gomeza, J. et al, Mol. Pharmacol; 1996, 50, 923-930.

Cells were plated in 96-well culture plates and labeled overnight with [³H]myoinositol. The day after, cells were washed three times with Krebs buffer, incubated for 10 min with LiCl 5 mM, and then incubated for 30 min in the absence (basal) or in the presence of the indicated compounds at 1 nM up to 1000 μM. The total amount of [³H]phosphatidylinositol accumulated in the cells was determined after Dowex purification as previously by Goudet C, et al, Prod. Natl. Acad. Sci. USA 2004, 101, 378-383.

The response dosis curves were adjusted by using equatrier y=[(Yₘₐₓ−yₘᵢₙ)/(1+(x/EC₅₀)ⁿ)]+yₘᵢₙ where EC₅₀ is the concentration necessary for obtaining half of the maximal effect and n is Hill coefficient.

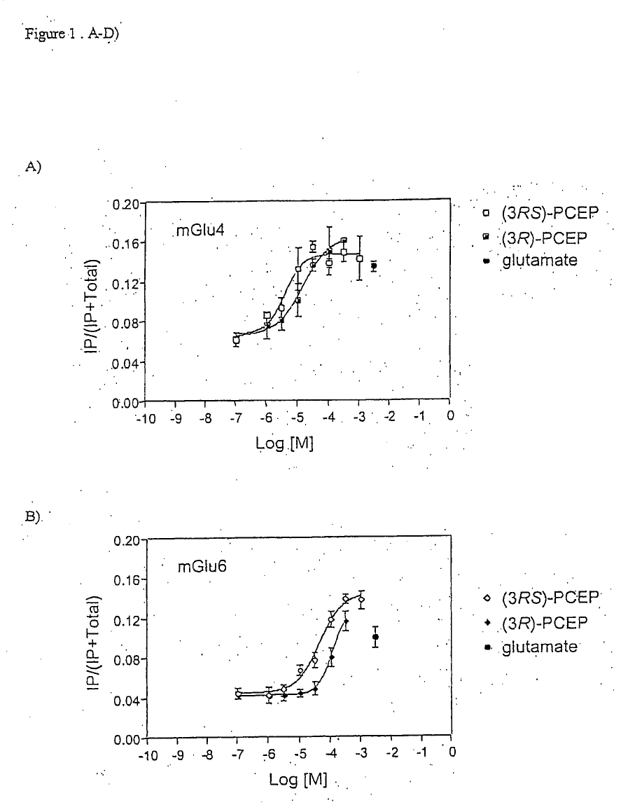

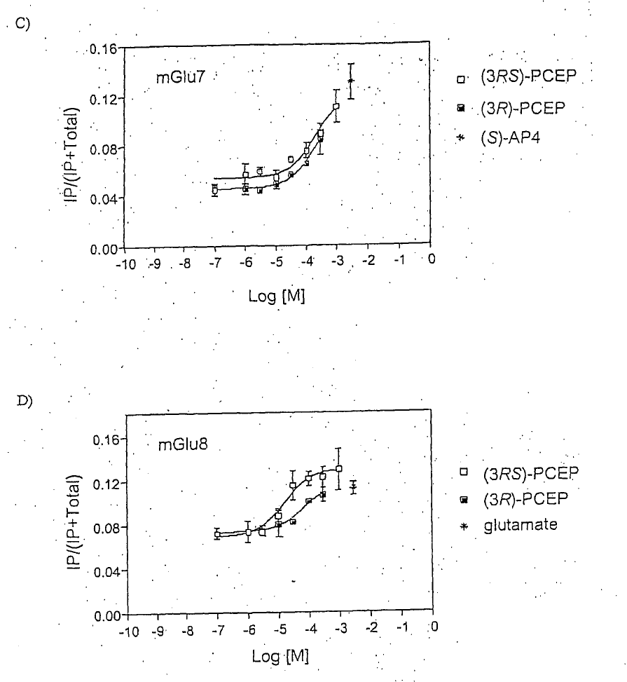

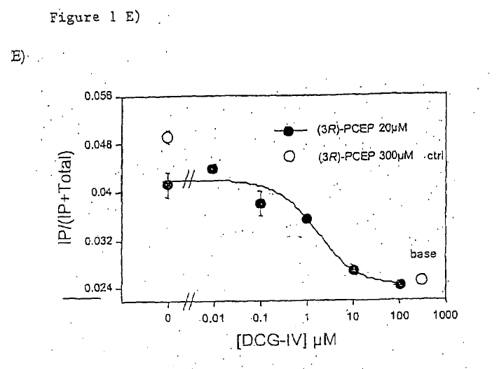

Results obtained with (3R)-PCEP and (3RS)-PCEP concerning the mGlu4, mGlu6, mGlu7 and mGlu8 receptors of group III are given in Table 1 and FIGS. 1A-1E.

TABLE 1

| Compounds | EC₅₀ mGlu4 μM | EC₆₀ mGlu6 μM | EC₅₀ mGlu7 μM | EC₅₀ mGlu8 μM |
|---|---|---|---|---|
| (3R)-PCEP | 24.2 ± 7.6 (3) | 99. ± 9.0 (3) | >1000 | 58.2 ± 8.8 (2) |
| (3SR)-PCEP | 6.6 ± 2.8 (2) | 33.1 ± 10 4 (2) | >1000 | 24.2 ± 9.0 (2) |

Results obtained with other compounds according to the invention are given in Table 2

TABLE 2

| | | | mGlu4 |
|---|---|---|---|
| Patent | Lab - | | EC₅₀ |
| Reference | Reference | structure | μM (n) |
| 4 | CS42 | 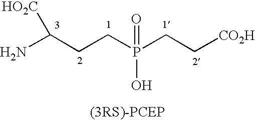 | RS 7.1 (5) R 24.2 (3) |
| 7 | CS80 CS2.071 | 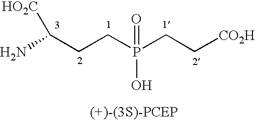 | 6.4 (5) |
| 11 | CS68 | 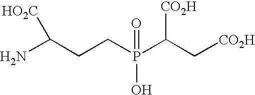 | 59.0 (5) |
| 14 | CS102 | 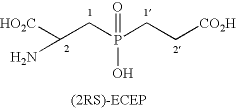 | inactive at 100 μM (1) . . . |
| 26 | CS128 | 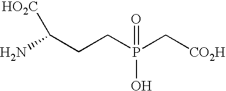 | 3.8 (2) |
| 19 | CS134 | 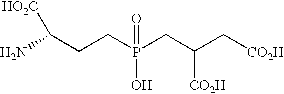 | 28.7 (2) |
| 60 | CS117 | 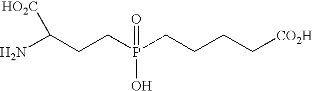 | inactive at 100 μM (1) |
| 31 | CS171 | 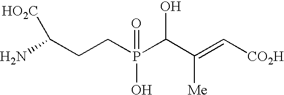 | inactive at 100 μM (1) |
| 24 | CS155 | 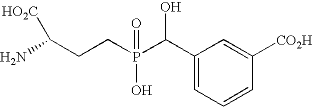 | 20.8 (5) |
| 22 | CS173 | 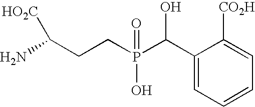 | inactive at 100 μM |
| 33-34 | CS158 | 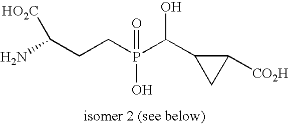 | 5.1 (2) |
| 33-34 | CS159 | 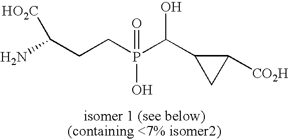 | 12.9 (2) partial |
| 36 | CS172 | 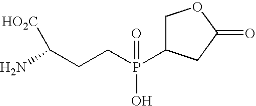 | 11.5 (2) partial |
| 16 | CS183 | 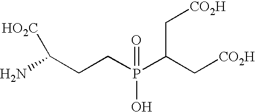 | 10% max at 100 μM (1) |
| 28 | CS191 | 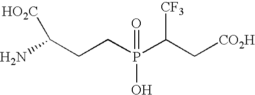 | 60% max at 100 μM (1) |
| 38 | CS2.012 | 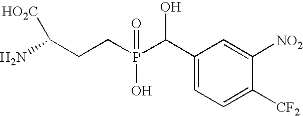 | 0.28 (9) |
| 40 | CS2.014 | 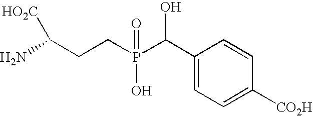 | 31.8 (3) |
| 42 | CS2.024 | 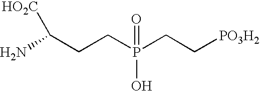 | 40% max at 100 μM (1) |
| 44 | CS2.029 | 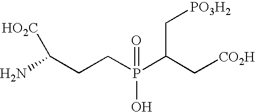 | 33% max at 100 μM (1) |
| 46 | CS2.041 | 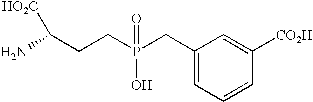 | 33% max at 100 μM (1) |
| 48 | CS2.042 | 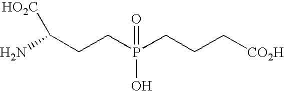 | 80% max at 100 μM (1) |
| 50 | CS2.080 | 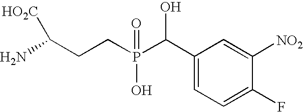 | 1.02 (7) |
| 52 | CS2.086 | 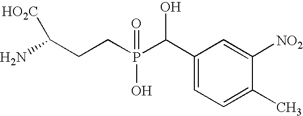 | 1.69 (5) |
| 54 | CS2.088 | 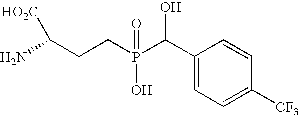 | 22.2 (3) |
| 56 | CS2.093 | 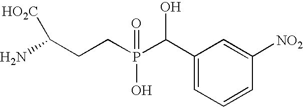 | 0.63 (14) |
| 62 | CS2.109 | 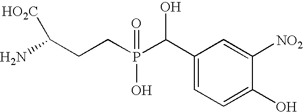 | 2.00 (4) |
| 58 | CS2.101 | 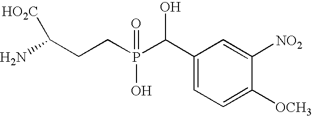 | 2.89 (3) |
| 66 | CS2.118 | 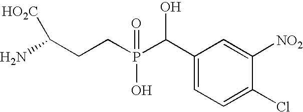 | 2.74 (2) |
| 64 | CS2.111 | 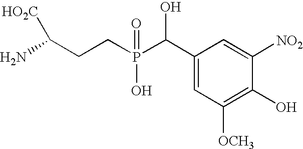 | 1.50 (4) |
| 68 | CS2123 | 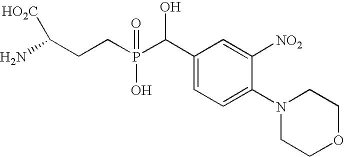 | >100 (3) |
| 70 | CS2.127 | 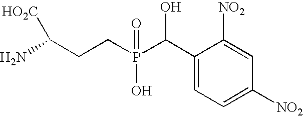 | 1.00 (4) |
| 72 | CS2.147 | 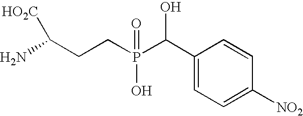 | 0.93 (2) |
| 74 | CS2.153 | 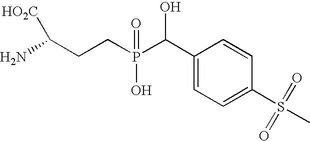 | >100 (2) |
| 76 | CS2.157 | 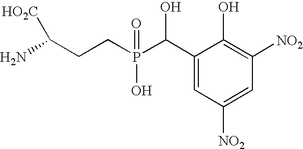 | 2.3 (3) |
| 78 | CS2.163 | 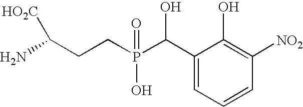 | 15.2 (2) |
| 82 | CS2.171 | 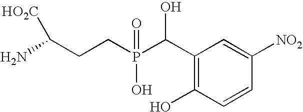 | 1.17 (4) |
| 90 | CS2.176 | 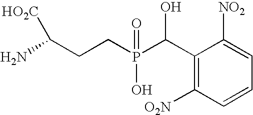 | 1.96 (4) |
| 80 | CS2.166 | 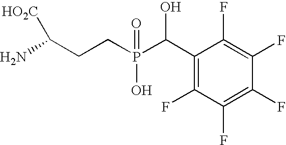 | 5.19 (4) |
| 86 | CS3.012 | 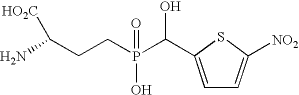 | 0.310 (4) |
| 84 | CS3.003 | 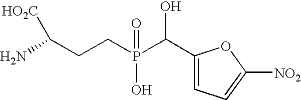 | 0.087 (6) |
| 97 | CS3.030 | 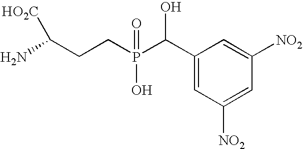 | 0.81 (3) |
| 94 | CS3.035 | 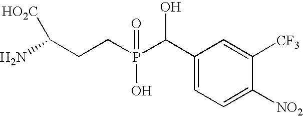 | 0.305 (2) |
| 88 | CS3.051 | 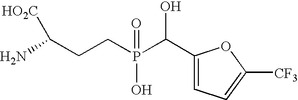 | 1.98 (2) |

CS158 and CS159 (33-34) are each a mixture of diastereoisomers.

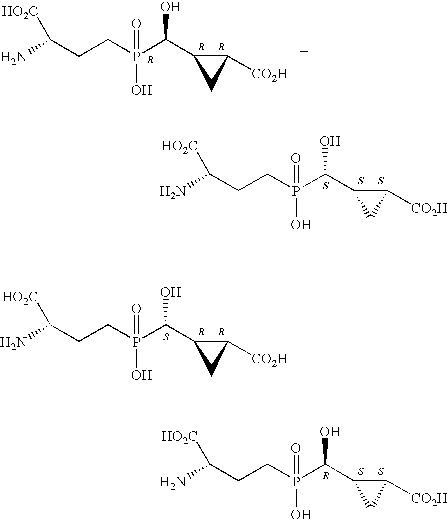

Antagonist activity of the compounds was tested on HEK293 cells transiently transfected with rat mGlu4 expressing plasmid pRKG4 and chimeric G-protein Gqi9 by electroporation, as described in (14)

Cells were plated in 96-well culture plates and labeled overnight with [3H]myoinositol. The day after, cells were washed three times with Krebs buffer, incubated for 10 min with LiCl nM, then pre-incubated for 5 min in the presence of the compounds at from 1 nM up to 1000 μM tested as an antagonist, and then incubated for 30 min in the presence or the absence of the agonist (L-AP4 from 0.1 to 100 μM, depending on the receptor tested mGlu4 (L-AP4 300 nM), mGlu6, mGlu7, mGlu8). Incubation was stopped by replacing the stimulation buffer by a solution of formic acid 0.1 M. The total amount of [3H]phosphatidylinositol accumulated in the cells was determined after Dowex purification as previously described in (15).

The response dosis curves were adjusted by using equatrier y=[(Ymax−y min)/(1+(x/EC50)n)]+ymin where IC50 is the concentration necessary for obtaining half of the maximal inhibitory effect and n is Hill coefficient.

The derivatives of the invention with antagonist properties are particularly useful for treating pathologies such as ADHD (Attention Deficit and Hyperactivity Disorder) and the so-called affective pathologies such as nervous breakdown and/or bipolar disorders (depressions followed by over excitation) and psychotic syndromes.

### REFERENCES

(1) Afzali-Ardakani, A.; Rapoport, H. L-Vinylglycine. J. Org. Chem. 1980, 45, 4817-4820.
(2) Olsen, J. A.; Severinsen, R.; Rasmussen, T. B.; Hentzer, M.; Givskov, M.; Nielsen, J. Synthesis of new 3- and 4-substituted analogues of acyl homoserine lactone quorum sensing autoinducers. Bioorg. Med. Chem. Lett. 2002, 12, 325-328.
(3) Krol, W. J.; Mao, S. S.; Steele, D. L.; Townsend, C. A. Stereochemical correlation of proclavaminic acid and syntheses of erythro- and threo-L-β-hydroxyornithine from an improved vinylglycine synthon. J. Org. Chem. 1991, 56, 728-731.
(4) Berkowitz, D. B.; Charette, B. D.; Karukurichi, K. R.; McFadden, J. M. α-vinylic amino acids: occurrence, asymmetric synthesis, and biochemical mechanisms. Tetrahedron: Asymmetry 2006, 17, 869-882.
(5) Cativiela, C.; Diaz-de-Villegas, M. D. Stereoselective synthesis of quaternary α-amino acids. Part 1: Acyclic compounds. Tetrahedron: Asymmetry 1998, 9, 3517-3599.
(6) Berkowitz, D. B.; Chisowa, E.; McFadden, J. M. Stereocontrolled synthesis of quaternary β,γ-unsaturated amino acids: chain extension of- and -α-(2-tributylstannyl)vinyl amino acids. Tetrahedron 2001, 57, 6329-6343.
(7) Seebach, D.; Juaristi, E.; Miller, D. D.; Schickli, C.; Weber, T. Addition of chiral glycine, methionine, and vinylglycine enolate derivatives to aldehydes and ketones in the preparation of enantiomerically pure-amino-hydroxy acids. Helv. Chim. Acta 1987, 70, 237-261.
(8) Weber, T.; Aeschimnann, R.; Maetzke, T.; Seebach, D. Methionin als Vorläufer zur enantioselektiven Synthese-verzweigter Vinylglycine und anderer Aminosäuren. Helv. Chim. Acta 1986, 69, 1365-1377.
(9) Acton, J. J.; Jones, A. B. Synthesis and derivatization of a versatile α-substituted lactam dipeptide isostere. Tetrahedron Lett. 1996, 37, 4319-4322.
(10) Annedi, S. C.; Biabani, F.; Poduch, E.; Mannargudi, B. M.; Majumder, K.; Wei, L.; Khayat, R.; Tong, L.; Kotra, L. P. Engineering D-amino acid containing novel protease inhibitors using catalytic site architecture. Bioorg. Med. Chem. 2006, 14, 214-236.
(11) Cheng, H.; Keitz, P.; Jones, J. B. Design and synthesis of a conformationally restricted cysteine protease inhibitor. J. Org. Chem. 1994, 59, 7671-7676.
(12) Ma, D.; Zhu, W. Synthesis of (S)-α-cyclopropyl-4-phosphonophenylglycine. J. Org. Chem. 2001, 66, 348-350.
(13) Groth, U.; Schöllkopf, U.; Chiang, Y.-C. Asymmetric syntheses via heterocyclic intermediates; XIII1. Enantioselective synthesis of (R)-α-alkenylalanine methyl esters using L-valine as chiral auxiliary reagent. Synthesis 1982, 864-866.
(14) Gomeza, J. et al, Mol. Pharmacol; 1996, 50, 923-930.
(15) Goudet C, et al, Prod. Natl. Acad. Sci. USA 2004, 101, 378-383.

1. Hypophosphorous acid derivatives having formula (I) 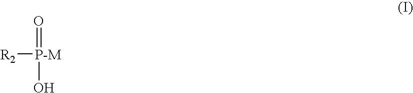
wherein
M is a [C(R₃,R₄)]ₙ₁—C(E,COOR₁,N(H,Z)) group, or an optionally substituted Ar—CH(COOR₁,N(H,Z)) group (Ar designating an aryl or an heteroaryl group), or an α, β cyclic aminoacid group such as, 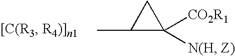
or a β,γ-cyclic aminoacid group such as 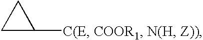
R₁ is H or R, R being an hydroxy or a carboxy protecting group, such as C₁-C₃ alkyl, Ar (being aryl or heteroaryl),
Z is H or an amino protecting group R′, such as C₁-C₃ alkyl, C₁-C₃ acyl, Boc, Fmoc, COOR, benzyl oxycarbonyl, benzyl or benzyl substituted such as defined with respect to Ar;
E is H or a C₁-C₃ alkyl, aryl, an hydrophobic group such as (CH₂)ₙ₁-aryl, (CH₂)ₙ₁-aryl (or heteroaryl), such as a benzyl group, or a xanthyl, alkyl xanthyl or alkyl thioxanthyl group, or
(CH₂)ₙ₁-cycloalkyl, —(CH₂)ₙ—(CH₂—Ar)₂, a chromanyl group, particularly 4-methyl chromanyle, indanyle, tetrahydro naphtyl, particularly methyl-tetrahydronaphtyl;
R₂ is selected in the group comprising:
D-CH(R₆)—C—(R₇, R₈)—
(R₁₁,R₁₂)CH—C(R₉, R₁₀)—
D-CH(OH)—
D-[C(R)₁₃,R₁₄)]ₙ₃—
C[(R₁₅,R₁₆,R₁₇)]ₙ₄—
D-CH₂—
(R₁₈)CH═C(R₁₉)—
D-(M₁)ₙ₆-CO— 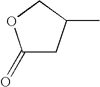
PO(OH)₂—CH₂ or (PO(OH)₂—CH₂), (COOH—CH₂)—CH₂—
with
D=H, OH, OR, (CH₂)ₙ₂OH, (CH₂)ₙ₁OR, COOH, COOR, (CH₂)ₙ₂COOH, (CH₂)ₙ₁COOR, SR, S(OR), SO₂R, NO, heteroaryl, C₁-C₃ alkyl, cycloalkyl, heterocycloalkyl, (CH₂)ₙ₂-alkyl, (COOH,NH₂)—(CH₂)ᵤ₁-cyclopropyl-(CH₂)ᵤ₂—, CO—NH-alkyl, Ar, (CH₂)ₙ₂—Ar, CO—NH—Ar, R being as above defined and Ar being an optionally substituted aryl or heteroaryl group,
R₃ to R₁₉, identical or different, being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂, 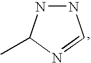
NO₂, SO₂NH₂, SO₂NHR; SR, S(O)R, SO₂R, benzyl;
one of R₁₁ or R₁₂ being COOR, COOH, (CH₂)n₂-COOH, (CH₂)n₂-COOR, PO₃H₂ the other one being such as defined for R₉ and R₁₀;
one of R₁₅, R₁₆ and R₁₇ is COOH or COOR, the others, identical or different, being such as above defined;
one of R₁₈ and R₁₉ is COOH or COOR, the other being such as above defined;
M₁ is an alkylene or arylene group;
n1=1, 2 or 3;
n2=1, 2 or 3,
n3=0, 1, 2 or 3 and
n4=1, 2 or 3;
n5=1, 2 or 3;
n6=0 or 1,
u1 and u2, identical or different=0, 1 or 2,
Ar, and alkyl groups being optionally substituted by one or several substituents on a same position or on different positions, said substituents being selected in the group comprising: OH, OR, (CH₂)ₙ₁OH, (CH₂)ₙ₁OR, COOH, COOR, (CH₂)ₙ₁COOH, (CH₂)ₙ₁COOR, C₁-C₃ allyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂, 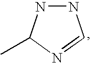
NO₂, SO₂NH₂, SO₂NH; SR, S(O)R, SO₂R, benzyl;
R being such as above defined,
with the proviso that formula I does not represent the racemic (3R,S) and the enantiomeric form (3R) of 3 amino,3-carboxy-propyl-2′-carboxy-ethylphosphinic acid; 3 amino,3-carboxy-propyl-4′carboxy,2′carboxy-butanoylphosphinic acid; 3 amino,3-carboxy-propyl-2′carboxy-butanoylphosphinic acid; 3 amino,3-carboxy-propyl-3′amino, 3′carboxy-propylylphosphinic acid; and 3 amino,3-carboxypropyl-7′amino-2′,7′-dicarboxyheptylphosphinic acid, said hypophosphorous acid derivatives being diasteroisomers or enantiomers.

2. The hypophosphorous acid derivatives of claim 1, having formula (II) 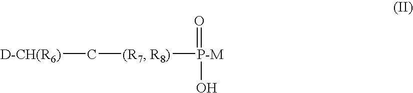
wherein the substituents are as above defined.

3. The hypophosphorous acid derivatives of claim 2, wherein D is Ar or a substituted Ar, especially a phenyl group having 1 to 5 substituents.

4. The hypophosphorous acid derivatives of claim 3, wherein the substituents are in ortho and/or meta and/or para positions and are selected in the group comprising OH, OR, (CH₂)ₙ₂OH, (CH₂)ₙ₂OR, COOH, COOR, (CH₂)ₙ₂COOH, (CH₂)ₙ₂COOR, C1-C3 alkyl or cycloalkyl, (CH₂)ₙ₂-alkyl, aryl, (CH)ₙ₂-aryl, halogen, CF₃, SO₃H, PO₃H₂, B(OH)₂ alkylamino, fluorescent group (dansyl, benzoyl dinitro 3, 5′, 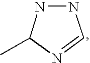
NO₂, SO₂NH₂, SO₂(NH,R)SR, S(O)R, SO₂R, OCF₃, heterocycle, heteroaryl, substituted such as above defined with respect to Ar.

5. The hypophosphorous acid derivatives of formula (III) 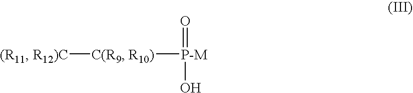
wherein the substituents are as above defined.

6. The hypophosphorous acid derivatives of claim 5, wherein one of R₁₁ or R₁₂ is COOH.

7. The hypophosphorous acid derivatives of claim 1, having formula (IV) 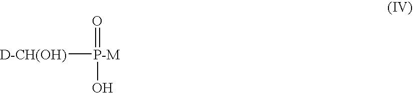
wherein the substituents are as above defined.

8. The hypophosphorous acid derivatives of claim 7, wherein D is as above defined with respect to formula II.

9. The hypophosphorous acid derivatives of claim 1, having formula (V) 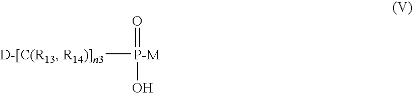
wherein the substituents are as above defined, one of R₁₃ or R₁₄ representing OH.

10. The hypophosphorous acid derivatives of claim 9, wherein D is as above defined with respect to formula II.

11. The hypophosphorous acid derivatives of claim 1, having formula (VI) 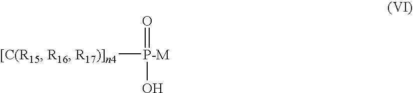
wherein the substituents are as above defined.

12. The hypophosphorous acid derivatives of claim 11, wherein, in the first group of the chain, one or two of R₁₅, R₁₆ or R₁₇ is COOH.

13. The hypophosphorous acid derivatives of claim 1, having formula (VII) 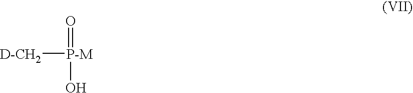
wherein the substituents are as above defined.

14. The hypophosphorous acid derivatives of claim 13, as above defined with respect to formula II.

15. The hypophosphorous acid derivative of claim 2, wherein R₆ to R₁₀, one of R₁₁ or R₁₂, one of R₁₃ or R₁₄, one or two of R₁₅, R₁₆ or R₁₇ is H, C₁-C₃ alkyl, OH, NH₂, CF₃.

16. The hypophosphorous acid derivatives of claim 1, having formula (VIII) 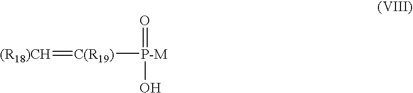
wherein the substituents are as above defined.

17. The hypophosphorous acid derivatives of claim 16, wherein R₁₈ is COOH.

18. The hypophosphorous acid derivatives of claim 16, wherein R₁₉ is H, C₁-C₃ alkyl, OH.

19. The hypophosphorous acid derivatives of claim 1, having formula LIX 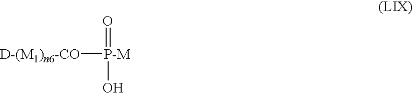
wherein the substituents are as above defined.

20. The hypophosphorous acid derivatives of claim 19, wherein either n6=0, or n6=1 and M₁ is an alkylene or an arylene group such as above defined.

21. The hypophosphorous acid derivatives of claim 1, where M is a [C(R₃,R₄)]ₙ₁—C(E,COOR₁,N(H,Z))group.

22. The hypophosphorous acid derivatives of claim 1, wherein M is an Ar group or a substituted arylene group, particularly a C6H4 group or a substituted C6H4 group, the substituents being as above defined with respect to formula I.

23. The hypophosphorous acid derivatives of claim 1, wherein M comprises a cyclic aminoacid group, particularly an α, β cyclic aminoacid group such as 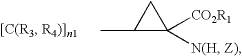
or a β,γ-cyclic aminoacid group such as

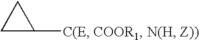

24. A process for preparing hypophosphorous acid derivatives of formula I 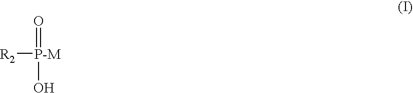
wherein the substituents are as above defined in claim 1, comprising
according to method A):
a1) treating a derivative of formula (IX) 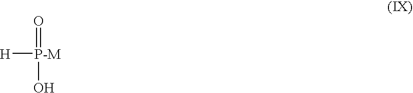
wherein the substituents and n1 are as above defined,
with either trimethylsilylchloride (TMSCl) and triethylamine (Et₃N), or N,O-(bis-triethylsilyl)acetamide (BSA);
a2) adding to the reaction product
one of the following derivatives having, respectively,
D-C(R₆)═C(R₇,R₈), or formula X
(R₁₁,R₁₂)C═C(R₉,R₁₀) formula XI
formula XII: 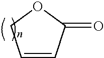
with n=1 or 2
D-CH(═O) formula XIII
D-[C(R₁₃,R₁₄)]ₙ₃—Br formula XIV
[C(R₁₅,R₁₆,R₁₇)]ₙ₄—Br formula XV
D-I formula XVI
(R₁₈)CH≡C(R₁₉) formula XVII
a3) treating the reaction product under acidic conditions or with catalysts to obtain the desired final product;
a4) recovering the diastereoisomers or the enantiomer forms,
a5) separating, if desired, diastereoisomers when obtained;
according to method B, said process comprises
b1) treating a derivative of formula (VIII)
(R″SiO)₂—P—H (XVIII)
wherein R″ is a C₁-C₃ alkyl
with
either a derivative of formula (X)
D-C(R₆)═C(R₇,R₈) (X)
or with a derivative of formula (XI)
(R₁₁,R₁₂)C═C(R₉,R₁₀) (XI)
wherein one of R₉ or R₁₀ is COOalk, alk being a C₁-C₃ alkyl
b2) treating the condensation product with a dibromo derivative of formula (XIX)
Br—[C(R₃,R₄)]ₙ₁—Br (XIX)
under reflux conditions; and adding HC(Oalk)₃
wherein alk is a C1-C3 alkyl
b3) treating the condensation product with a derivative of formula (XX)
NH(Z)-CH(CO₂R)₂ (XX)
in the presence of K₂CO₃, BuO₄NBr, under reflux conditions;
b4) treating the condensation product under acidic conditions or with catalyst to obtain the final desired product;
b5) recovering the diastereoisomers or the enantiomer forms, and
b6) if desired, separating diastereoisomers, when obtained, into the enantiomers; or
alternatively, the reaction product obtained at step b1) is reacted, according to step b2i), with a derivative of formula (XXI)
[(R₃,R₄)C]ₙ₁═C(COOR₁,NH(Z)) (XXI)
and, according to step b3i), the reaction product is treated under acidic conditions to give the final desired product.
according to method C, said process comprises
c1) reacting, as defined in step a1), a derivative of formula (XXII) 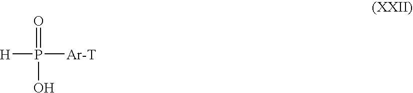
wherein Ar is as above defined and preferably an optionally substituted C₆H₄ group and T represents a C₁-C₃ alkyl group
c2) carrying out reaction step a2) by using one of the derivatives of formula (X) to (XVII)
c3) treating the reaction product with NBS, AiBN to have a bromo derivative with Ar substituted by T′-Br, with T′=CH₂
c4) reacting the bromo derivative thus obtained with (CH)₆N₄ in an organic solvent, then AcOH/H₂O to obtain a cetone derivative with Ar substituted by —C═O,
c5) treating cetone derivatives with KCN, NH₄Cl and NH₄OH to obtain aminocyano derivatives, with Ar substituted by —C(CN,NH₂)
c6) treating under acidic conditions to obtain derivatives with Ar substituted by —C(COOR,NH₂), and
c7) treating with catalysts to obtain the final desired product.

25. The process of claim 24, wherein
in method A, according to a preferred embodiment
the use of derivatives of formula (X)
D-CH(R₈)═C(R₇,R₈) (X)
with derivatives of formula (IX) results, in step a2), in intermediate derivatives of formula 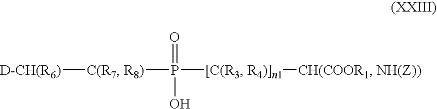
and, in step a3), in a final product of formula (XXIV) 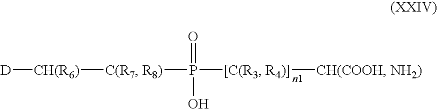
the use of derivatives of formula (XI) or formula (XII) 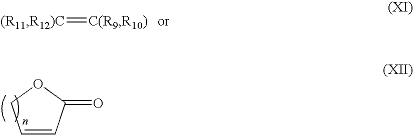
results, in step a2), in intermediate derivatives of formula (XXV) 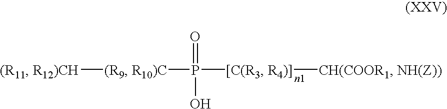
and, in step a3), in a final product of formula (XXVI) 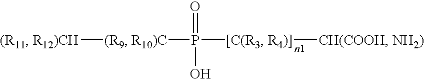
the use of derivatives of formula (XIII)
D-CH(═O) (XIII)
results, in step a2), in intermediate derivatives of formula (XXVII) 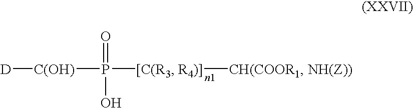
and, in step a3), in a final product of formula (XXVIII) 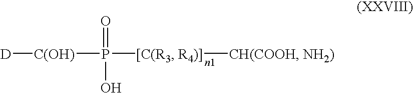
the use of derivatives of formula (XIV)
D-[C(R₁₃,R₁₄)]ₙ₃—Br (XIV)
results, in step a2), in intermediate derivatives of formula (XXIX) 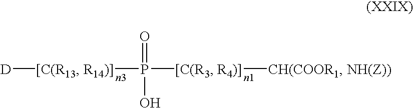
and, in step a3), in a final product of formula (XXX) 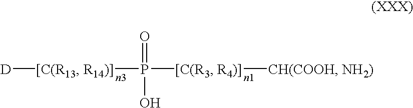
the use of derivatives of formula (XV)
[C(R₁₅,R₁₆,R₁₇)]ₙ₄—Br (XV)
results, in step a3), in intermediate derivatives of formula (XXXI) 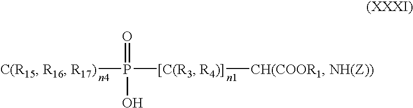
and, in step a3), in a final product of formula (XXXII) 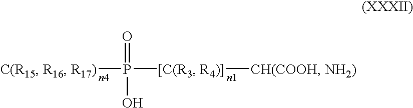
the use of derivatives of formula (XVI)
D-I (XVI)
results, in step a2), in intermediate derivatives of formula (XXXIII) 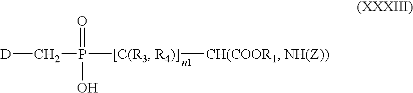
and, in step a3), in a final product of formula (XXXIV) 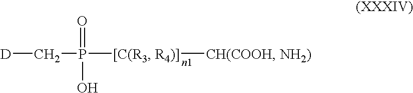
the use of derivatives of formula (XVII)
(R₁₈)C≡C(R₁₉) (XVII)
results, in step a2), in intermediate derivatives of formula (XXXV) 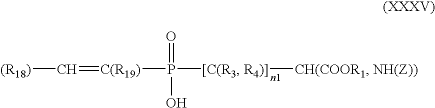
and, in step a3), in a final product of formula (XXXVI) 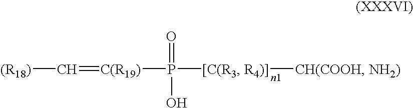
the use of derivatives of formula (LIX) 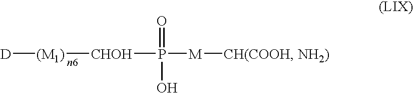
wherein M₁ is as above defined with respect to M and results by oxidation in a product of formula (LXI)

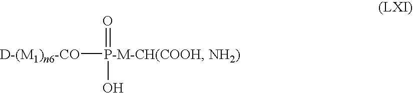

26. The method of claim 24, wherein
in method B,
the use, with derivatives of formula (XVIII), of derivatives of formula (X)
D-CH(R₈)—C(R₇,R₈) (X)
results, in step b1), in intermediate derivatives of formula (XXXVII)
D-CH(R₆)—C(R₇,R₈)—P—(OSiR″)₂ (XXXVII)
in step b2), in intermediate derivatives of formula (XXXVIII) 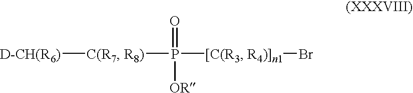
in step b3), in intermediate derivatives of formula (XXXIX) 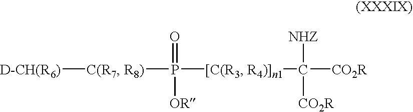
and, in step b4), in a final product of formula (XXXX) 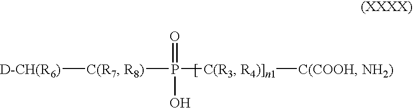
the use, with derivatives of formula (XVIII), of derivatives of formula (XI)
(R₁₁,R₁₂)C═C(R₉,R₁₀) (XI)
results, in step b1), in intermediate derivatives of formula (XXXXI)
(R₁₁,R₁₂)CH—C(R₉,R₁₀)—P—(OSiR″)₂ (XXXXI)
in step b2), in intermediate derivatives of formula (XXXXII) 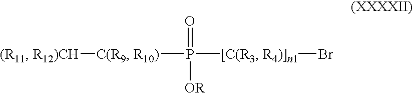
in step b3), in intermediate derivatives of formula (XXXXIII) 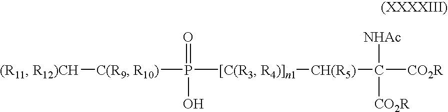
in step b4), in final products of formula (XXXXIV) 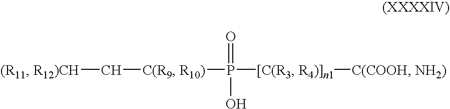
or, alternatively,
the use with derivatives of formula (XXXXI) obtained according to step b1) is reacted with a derivative of formula (XXXXV)
[(R₃,R₄)C]ₙ₁═C(COOR,NH(Z) (XXXXV)
giving intermediate derivatives of formula (XXXXVI) 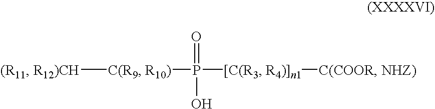
the treatment under acidic conditions giving the final product of formula (XXXXVII)

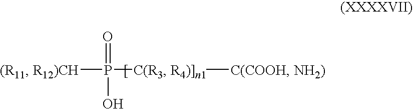

27. The process of claim 24, wherein
in method C,
the use, of a derivative of formula (XXII), 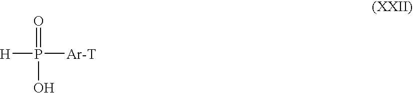
with a derivative of
D-C(R₆)═C(R₇,R₈), or formula X
(R₁₁,R₁₂)C═C(R₉,R₁₀) formula XI
formula XII: 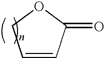
D-CH(═O) formula XIII
D-[C(R₁₃,R₁₄)]ₙ₃—Br formula XIV
[C(R₁₅,R₁₆,R₁₇)]ₙ₄—Br formula XV
D-I formula XVI
(R₁₈)C≡C(R₁₉) formula XVI
results in intermediate derivatives respectively having formulae (XXXXVIII) to (LIV)

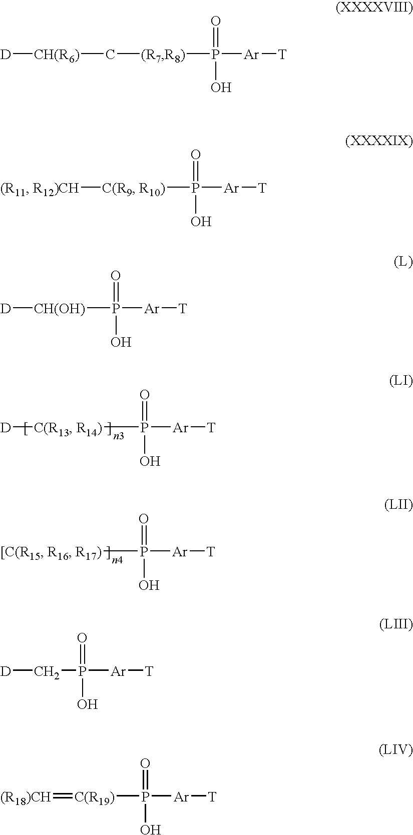

28. The process of claim 24, wherein
in method A, the derivatives of formula IX 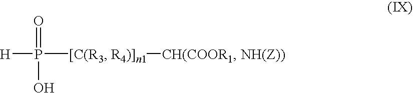
are advantageously obtained by reacting hypophosphorous acid of formula (LV) 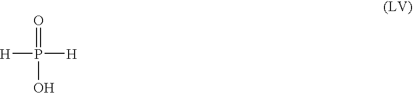
with a derivative of formula (LVI)
(R₃,R₄)ₙ₁C═CH—C(E,COOR₁,NH(Z)) (LVI)
preferably Z-vinyl-glyOMe or a derivative thereof with E different from H,
the reaction being advantageously carried out in the presence of AIBN by heating above 50° C.-100° C., preferably at about 90° C.

29. The process of claim 24, wherein
in method B, the derivatives of formula (XVIII)
(R″SiO)₂—P—H (XVIII)
are obtained by reacting an hypophosphorous acid ammonium salt of formula (LVII) 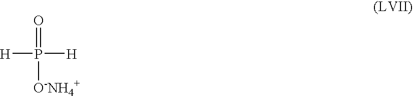
with hexamethyl disilazane of formula (LVIII)
(alk₃Si)—NH (LVIII)
the reaction being carried under an inert gas, by heating above 100° C., particularly at about 120° C.,
or by reacting hypophosphorous acid with N,O-(bis-triethylsilyl)acetamide (BSA) at room temperature.

30. The method of claim 24, wherein
in method C, the derivatives of formula (XXII) 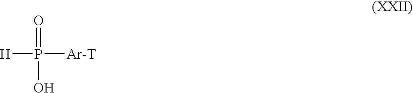
are advantageously obtained by reacting a mixture of H₃PO₂, Ar—NH₂, Ar—Br and a catalyst Pd(0) Ln (Ln=n ligands).

31. Hypophosphorous acid derivatives which are intermediates in the process of claim 24.

32. Pharmaceutical compositions comprising an effective amount of at least one of the hypophosphorous acid derivatives according to claim 23 with a carrier.

33. The pharmaceutical compositions according to claim 32, which are under a form suitable for an administration by the oral route, such as tablets, pills or capsules.

34. The pharmaceutical compositions of claim 33, comprising 1 to 100 mg of active ingredient per dose unit.

35. The pharmaceutical compositions according to claim 32, which are under a form suitable for an administration by injection, such as injectable solutions for the intravenous, subcutaneous or intramuscular route.

36. The pharmaceutical compositions of claim 35, comprising 1 to 30 mg of active ingredient per dose unit.

37. The pharmaceutical composition of claim 32 for treating convulsions, pain, drug addiction, anxiety disorders and neurodegenerative diseases.

38. (canceled)

39. A method of treatment of brain disorders, comprising administering to a patient in need thereof an effective amount of an hypophosphorous acid derivative according to claim 1.
